(19) United States
(12) Patent Application Publication
(10) Pub. No.: US 2019/0367625 A1
(43) Pub. Date: Dec. 5, 2019

(54) AMHRII-BINDING COMPOUNDS FOR PREVENTING OR TREATING CANCERS

(71) Applicants: GAMAMABS PHARMA, Toulouse (FR); INSTITUT CURIE, Paris (FR)

(72) Inventors: Jean-Marc BARRET, CASTRES (FR); Jean-François PROST, VERSAILLES (FR); Mehdi LAHMAR, VIROFLAY (FR); Stéphane DEGOVE, PANTIN (FR); Olivier DUBREUIL, MAURESSAC (FR); André NICOLAS, PARIS (FR); Didier MESURE, PARIS (FR)

(21) Appl. No.: 16/434,232

(22) Filed: Jun. 7, 2019

Related U.S. Application Data
(63) Continuation-in-part of application No. PCT/EP2018/059548, filed on Apr. 13, 2018.

(30) Foreign Application Priority Data
Apr. 14, 2017 (EP) 17305445.3

(51) Int. Cl. C07K 16/28; A61P 35/00; A61K 47/68; A61K 38/07; A61K 35/17; A61K 39/395; A61K 31/7072; A61K 31/513; C12Q 1/6886

(52) U.S. Cl. CPC C07K 16/2869; A61P 35/00; A61K 47/6849; A61K 47/6811; A61K 38/07; A61K 2039/505; A61K 39/3955; A61K 31/7072; A61K 31/513; C12Q 1/6886; C07K 2317/565; A61K 35/17

(57) ABSTRACT

The present invention relates to a human AMHRII-binding agent for its use for preventing or treating a cancer selected in a group of cancers comprising colon cancer, liver cancer, hepatocellular carcinoma, testis cancer, thyroid cancer, gastric cancer, gastrointestinal cancer, bladder cancer, pancreatic cancer, head and neck cancer, kidney cancer, liposarcoma, pleuramesothelioma, melanoma, sarcoma, brain cancer, osteocarcinoma and leukemia.

### FIELD OF THE INVENTION

The present invention relates to the field of cancer treatment.

### BACKGROUND OF THE INVENTION

One of the main causes of death in world population is cancer or malignant tumor, wherein the mortality rates rank order is lung cancer, gastric cancer, liver cancer, colorectal cancer, breast cancer and cervical cancer. One-third of all individuals in the United States alone will develop cancer. Although the five-year survival rate has risen dramatically nearly fifty percent as a result of progress in early diagnosis and therapy, cancer still remains second only to cardiac disease as a cause of death in the United States. Twenty percent of Americans die from cancer, half due to lung, breast, and colon-rectal cancer. Moreover, skin cancer remains a health hazard.

Designing effective treatments for patients with cancer has represented a major challenge. The current regimen of surgical resection, external beam radiation therapy, and/or systemic chemotherapy has been partially successful in some kinds of malignancies, but has not produced satisfactory results in others. Furthermore, these approaches often have unacceptable toxicity.

Both radiation and surgery suffer from the same theoretical drawback. It has been recognized that, given that a single clonogenic malignant cell can give rise to sufficient progeny to kill the host, the entire population of neoplastic cells must be eradicated. See generally, Goodman and Gilman The Pharmacological Basis of Therapeutics (Pergamon Press, 8th Edition) (pp. 1202-1204). This concept of “total cell kill” implies that total excision of a tumor is necessary for a surgical approach, and complete destruction of all cancer cells is needed in a radiation approach, if one is to achieve a cure. In practice this is rarely possible; indeed, where there are metastases, it is impossible.

Moreover, traditional chemotherapeutic cancer treatments also rarely result in complete remission of the tumor, and the significant dosage levels required to generate even a moderate response are often accompanied by unacceptable toxicity. Anticancer agents typically have negative hematological effects (e.g., cessation of mitosis and disintegration of formed elements in marrow and lymphoid tissues), and immunosuppressive action (e.g., depressed cell counts), as well as a severe impact on epithelial tissues (e.g., intestinal mucosa), reproductive tissues (e.g., impairment of spermatogenesis), and the nervous system. P. Calabresi and B. A. Chabner, In: Goodman and Gilman The Pharmacological Basis of Therapeutics (Pergamon Press, 8th Edition) (pp. 1209-1216). The high dosage levels, and the resulting toxicity, are in large part necessitated by the lack of target specificity of the anticancer agents themselves. The drug needs to distinguish between host cells that are cancerous and host cells that are not cancerous. The vast bulk of anticancer drugs are indiscriminate at this level, and have significant inherent toxicity. Anticancer armamentarium has recently been enriched with immunotherapies know as checkpoint inhibitors. Those products (anti-PD1, anti-PDL1, anti-CTLA4) are able to unlock the immune system by counteracting the mechanisms by which cancer cells evade from immune surveillance and cell killing. Despite the fact that these products led to remarkable long term results in several cancers (like melanoma and lung cancer), the percentage of responders remains low to moderate and their spectrum of indications remains relatively restricted (D M. Pardoll, Nature Review 2012)

There is still a need for alternative or complementary anti-cancer therapies to the conventional surgical therapies, radiation therapies and chemotherapies. One of such promising alternative or complementary therapies has consisted in specifically targeting cancer cells through the recognition of antigens expressed by tumor cells by therapeutic agents. In 2017, such tumor cell-specific therapeutic strategies are mainly illustrated by antibody-based therapy bispecific antibodies and CAR-T cell-based therapy which can be engineered to increase immune cell engagement such as NK and macrophages (like glyco-engineered antibodies) or such as killer T-lymphocytes (like CD3 bispecific formats). Antibodies can also be armed by various cytotoxic agents under the format of Antibody Drug Conjugate (ADCs). Finally, T-cells themselves can be genetically engineered to directly recognize tumor cell and activate TCR signaling (CAR-T cells). The most those agents are potent the most the demand for tumor selective targets is increased.

Antibody-based therapy for cancer has become established over the past 15 years and is now one of the most successful and important strategies for treating patients with haematological malignancies and solid tumours. A key challenge has been to identify antigens that are suitable for antibody-based therapeutics. Such therapeutics can function through mediating alterations in antigen or receptor function (such as agonist or antagonist functions), modulating the immune system (for example, changing Fc function and T cell activation) or delivering a specific drug that is conjugated to an antibody that targets a specific antigen (Van den Eynde, B. J. & Scott, A. M. Encyclopedia of Immunology (eds Roitt, D. P. J. & Roitt, I. M.) 2424-2431 (Academic Press, London, 1998), Scott, A. M. et al. A Phase I clinical trial with monoclonal antibody ch806 targeting transitional state and mutant epidermal growth factor receptor. Proc. Natl Acad. Sci. USA 104, 4071-4076 (2007), Hughes, B. Antibody—drug conjugates for cancer: poised to deliver? Nature Rev. Drug Discov. 9, 665-667 (2010), Weiner, L. M., Surana, R. & Wang, S. Monoclonal antibodies: versatile platforms for cancer immunotherapy. Nature Rev. Immunol. 10, 317-327 (2010).). Molecular techniques that can alter antibody pharmacokinetics, effector function, size and immunogenicity have emerged as key elements in the development of new antibody-based therapies. Evidence from clinical trials of antibodies in cancer patients has revealed the importance of iterative approaches for the selection of antigen targets and optimal antibodies, including the affinity and avidity of antibodies, the choice of antibody construct, the therapeutic approach (such as signaling abrogation or immune effector function) and the need to critically examine the pharmacokinetic and pharmacodynamic properties of antibodies in early clinical trials. This Review summarizes the steps that are necessary to transform monoclonal antibodies (mAbs) into reagents for human use, the success of antibodies in the treatment of cancer patients, the challenges in target and construct selection, and the crucial role of the immune system in antibody therapy. Since the first commercialization of a therapeutic monoclonal antibody in 1986, this class of biopharmaceutical products has grown significantly so that, as of the end of 2014, forty seven monoclonal antibodies have been approved in the United States or in Europe, especially for the treatment of cancers. It is expected that about 70 monoclonal antibodies will be on the market by 2020.

CAR-T-Cell therapy is based on the manufacture of chimeric antigen T-cell receptors (CARs). Chimeric antigen receptors are genetically engineered receptors which graft a new specificity onto an immune effector cell. These are typically used to graft the specificity of a monoclonal antibody onto a T-cell. CAR-T cells are under investigation as a therapy for cancer. Typically, a CAR-T therapy involves infusion of engineered T-cells that express a Chimeric Antigen Receptor on their cell membrane. This receptor comprises an external target-binding domain which is designed to recognize a specific tumor antigen and an internal activation domain responsible for activating the T-cell when the CAR-T binds the antigen target. CAR-T clinical trials for treating cancers have shown huge remission rates, of up to 94% in severe forms of cancer, which is particularly impressive considering most of the trials recruit patients that have not responded to all other available treatments for their form of cancer. Until 2017, about 300 CAR-T clinical trials have been performed

There is still a need in the art for further tools for the therapy of cancers, that may be alternative or complementary the existing therapies for treating specific kind of cancers.

### SUMMARY OF THE INVENTION

This invention relates to a human AMHRII-binding agent for use in a method for preventing or treating non-gynecologic cancers.

Especially, this invention relates to a human AMHRII-binding agent for use in a method for preventing or treating non-gynecologic cancers selected in a group of cancers comprising colon cancer, liver cancer, hepatocellular carcinoma, testis cancer, thyroid cancer, gastric cancer, gastrointestinal cancer, bladder cancer, pancreatic cancer, head and neck cancer, kidney cancer, liposarcoma, fibrosarcoma, pleuramesothelioma, melanoma, sarcoma, brain cancer, osteocarcinoma, breast cancer, prostate cancer and leukemia. Colon cancer encompasses colorectal carcinoma. Kidney cancer encompasses renal cell carcinoma.

In some embodiments, the said human AMHRII-binding agent consists of an anti-AMHRII monoclonal antibody.

In some embodiments, the said human AMHRII-binding agent consists of an Antibody Drug Conjugate (ADC).

In some embodiments, the said human AMHRII-binding agent may be used in a combined anti-cancer treatment also comprising another anti-cancer agent. In some embodiments, the said other anti-cancer agent is active against metastatic colorectal cancer. In some embodiments, the said other anti-cancer agent comprises a nucleotide analogue, optionally combined with a nucleotide analogue degradation inhibitor such as a thymidine phosphorylase inhibitor. In some embodiments, the said other anti-cancer agent consists of a combination of trifluridine and tiripacil.

In some embodiments, the said human AMHRII-binding agent consists of an AMHRII-binding engineered receptor.

In some embodiments, the said human AMHRII-binding agent consists of a cell expressing an AMHRII-binding engineered receptor, such as a CAR T-cell or a NK T-cell expressing an AMHRII-binding engineered receptor.

This invention also pertains to a method for determining whether an individual is eligible to a cancer treatment with an AMHRII-binding agent as defined above, i.e. whether an individual is responsive to a cancer treatment with an AMHRII-binding agent as defined above, wherein the said method comprises the step of determining whether a tumor tissue sample previously obtained from the said individual express the AMHRII protein at the cell surface.

Thus, this invention also pertains to a method for determining whether an individual is responsive to a cancer treatment with an AMHRII-binding agent as defined above, wherein the said method comprises the step of determining whether a tumor tissue sample previously obtained from the said individual express the AMHRII protein at the cell surface.

### DESCRIPTION OF THE FIGURES

FIG. 1A-B illustrates the amino acid sequences of the VH and VL domains of a plurality of variants of the 3C23 monoclonal antibody. FIG. 1A illustrates the VH domain of each antibody variant. FIG. 1B illustrates the VL domain of each antibody variant.

FIG. 2A-F illustrates AMHRII expression by various cancer cell lines.

FIG. 2A illustrates the AMHRII mRNA expression by cancer cell lines. Abscissa: from the left to the right of FIG. 2A: HCT116 (colon colorectal carcinoma), COV434-WT (human ovarian granulosa tumor), K562 (human myelogenous leukemia) and OV90 (human malignant papillary serous adenocarcinoma). Ordinate: AMHRII mRNA expression level as assayed by RT-qPCR, expressed in Arbitrary Units (RQ).

FIGS. 2B to 2F: AMHRII protein membrane expression by the same cancer cell lines as in FIG. 2A: HCT116 (FIG. 2B), COV434-WT (FIG. 2C), K562 (FIG. 2D), NCI-H295R (FIG. 2E) and OV90 (FIG. 2F). Abscissa: fluorescence signal intensity (FL2-A dye) as expressed in Arbitrary Units. Ordinate: cell count.

FIG. 3 illustrates the AMHRII surface expression in various human tumor primary tissue samples. Abscissa: type of cancer; from the left to the right of FIG. 3: colon cancer, liver cancer, testis cancer, thyroid cancer, gastric cancer, bladder cancer, pancreatic cancer, head and neck cancer. Ordinate: AMHRII positivity index was defined by an AMHRII global score ≥1.5. This Global histological score was established by the mean of cytoplasmic+membranous score. Each of these scores using frequency×mean of intensity scores (0 to 3). Frequency was defined as a percentage of cells expressing AMHRII and intensity was classified as unequivocal brown labeling of tumor cell membrane or cytoplasm through the following scoring system: intensity of the labeling was defined as 0 for negative, 1 for weak, 2 for moderate, and 3 for strong as shown in the COV434 positive control; Numbers located above each bar: frequency of AMHRII expression for the corresponding cancer in the tested human population.

FIG. 4 illustrates the AMHRII surface expression by various human tumor xenografts. Abscissa, from the left to the right of FIG. 4: leukemia, osteosarcoma, gastrointestinal cancer, brain cancer, sarcoma, melanoma, pleuramesothelioma, liposarcoma, testis cancer, colon cancer, kidney cancer. Ordinate: AMHR2 global score, as expressed in ARbitrary Units.

FIG. 5 illustrates the in vivo anti-tumor activity of the 3C23K antibody against a PDX model human hepatocarcinoma (HCC). Abscissa: Time period following the beginning of the treatment, as expressed in days. Ordinate: tumor volume, as expressed in mm³. ●:vehicle; ▴ 3C23K antibody at the dose of 20 mg/kg; ▪: 3C23K antibody at the dose of 50 mg/kg s; ▾: comparative treatment with sorafenib at the dose of 50 mg/kg. Ordinate: Tumor Volume as expressed in mm³. Abscissa: ●Vehicle; ▴ 3C23K antibody at the dose of 20 mg/kg; ▪ 3C23K antibody at the dose of 50 mg/kg; ▾ Sorafenib at the dose of 50 mg/kg.

FIG. 6 illustrates the in vivo anti-tumor activity of the Antibody Drug Conjugate (ADC) consisting of a 3C23K antibody cytotoxic conjugate (termed GM103) as disclosed in the PCT application no WO 2017/025458 against a PDX model human hepatocarcinoma (HCC). Abscissa: Time period following the beginning of the treatment, as expressed in days. Ordinate: tumor volume, as expressed in mm³. ●:vehicle; ▾ GM103 ADC at the dose of 1 mg/kg; ▴: GM103 ADC at the dose of 5 mg/kg; ▪ GM103 ADC at the dose of 10 mg/kg;

FIG. 7A-D illustrates AMHRII membrane expression by tumor cells originating from tumor samples from four patients (FIGS. 7A; 7B, 7C, 7D) affected with a colorectal cancer, as measured by flow cytometry (FACS). Abscissa: fluorescence signal intensity (FL2-A dye) as expressed in Arbitrary Units. Ordinate: cell count. In FIGS. 7A, 7B, 7C, 7D: (i) peak on the left side: cells incubated with an unrelated isotype antibody; (ii) peak on the right sides: cells incubated with the 3C23K anti-AMHRII antibody.

FIG. 8A-D: illustrates AMHRII membrane expression by four distinct colorectal cancer human xenografts (FIGS. 8A, 8B, 8C, 8D) in mice, as measured by flow cytometry (FACS). Abscissa: fluorescence signal intensity (FL2-A dye) as expressed in Arbitrary Units. Ordinate: cell count. In FIGS. 8A, 8B, 8C, 8D: (i) peak on the left side: cells incubated with an unrelated isotype antibody; (ii) peak on the right sides: cells incubated with the 3C23K anti-AMHRII antibody.

FIG. 9A-B: illustrates AMHRII membrane expression by tumor cells originating from tumor samples from two patients (FIGS. 9A; 9B) affected with a renal cell carcinoma, as measured by flow cytometry (FACS). Abscissa: fluorescence signal intensity (FL2-A dye) as expressed in Arbitrary Units. Ordinate: cell count. In FIGS. 9A, 9B: (i) peak on the left side: cells incubated with an unrelated isotype antibody; (ii) peak on the right sides: cells incubated with the 3C23K anti-AMHRII antibody.

FIG. 10 illustrates the in vivo anti-tumor activity of the anti-AMHRII antibody GM102 against a PDX model of human colorectal carcinoma (CRC). Abscissa: Time period following the beginning of the treatment, as expressed in days. Ordinate: tumor volume, as expressed in mm³. ●:vehicle; ▪ GM102 at the dose of 20 mg/kg; ▴: Irinotecan at the dose of 100 mg/kg.

FIG. 11 illustrates the level of membranous expression of AMHRII (as measured by Immuno-histo-chemistry) by the tumor cells from patients who have received a combined treatment with (i) the 3C23K anti-AMHRII antibody and (ii) Lonsurf comprising the combination of trifluridine and tipiracil. Abscissa: left part: stabilized patients (“Stab+”); right part: non-stabilized patients (“Stab−”). Ordinate: level of AMHRII expression, as expressed in percent of AMHRII positive cells for membranous expression.

FIG. 12 illustrates the level of membranous expression of AMHRII by the tumor cells from patients who have received a combined treatment with (i) the 3C23K anti-AMHRII antibody and (ii) Lonsurf comprising the combination of trifluridine and tipiracil. Abscissa: left part: mean value of the whole cohort of patients; middle part: non-stabilized patients (“Stab−”); right part: stabilized patients (“Stab+”). Ordinate: level of AMHRII expression, as expressed in membranous score value. The membranous score value is determined as follows: signal intensity×% AMHRII-positive cells.

FIG. 13 illustrates a graph showing the correlation between (A) the duration of the treatment of patients with (i) the 3C23K anti-AMHRII antibody and (ii) Lonsurf comprising the combination of trifluridine and tipiracil and (B) the level of AMHRII expression as determined by the membranous score value by immuno-histo-chemistry. Abscissa: treatment duration, as expressed in months; Ordinate: level of AMHRII expression, as expressed as the membranous score value. Correlation analysis was performed according to the Spearman r test at 95% confidence interval.

FIG. 14 illustrates a graph showing (A) the duration of the treatment of patients with (i) the 3C23K anti-AMHRII antibody and (ii) Lonsurf comprising the combination of trifluridine and tipiracil as a function of (B) the level of AMHRII expression as determined by the membranous score value. Abscissa; left part, treated patients having an AMHRII membranous score >20; right part: treated patients having an AMHRII membranous score <20. Ordinate: treatment duration, as expressed in months. In FIG. 14, the comparison was performed between treatment durations of 4.2 months and 2.3 months, respectively.

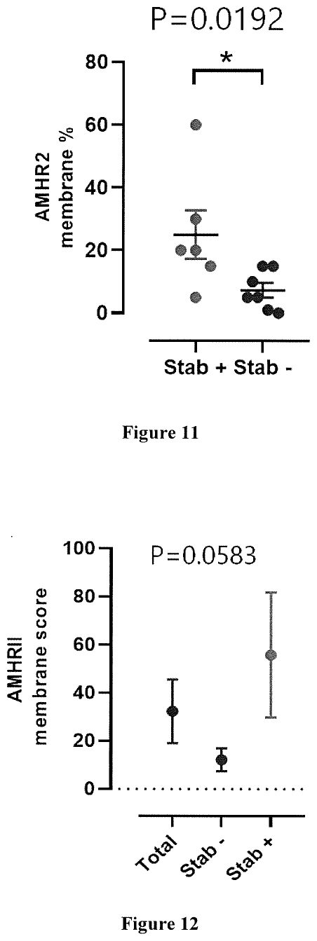

FIG. 11 illustrates the level of membranous expression of AMHRII (as measured by Immuno-histo-chemistry) by the tumor cells from patients who have received a combined treatment with (i) the 3C23K anti-AMHRII antibody and (ii) Lonsurf comprising the combination of trifluridine and tipiracil. Abscissa: left part: stabilized patients (“Stab+”); right part: non-stabilized patients (“Stab−”). Ordinate: level of AMHRII expression, as expressed in percent of AMHRII positive cells for membranous expression.

FIG. 12 illustrates the level of membranous expression of AMHRII by the tumor cells from patients who have received a combined treatment with (i) the 3C23K anti-AMHRII antibody and (ii) Lonsurf comprising the combination of trifluridine and tipiracil. Abscissa: left part: mean value of the whole cohort of patients; middle part: non-stabilized patients (“Stab−”); right part: stabilized patients (“Stab+”). Ordinate: level of AMHRII expression, as expressed in membranous score value. The membranous score value is determined as follows: signal intensity×% AMHRII-positive cells.

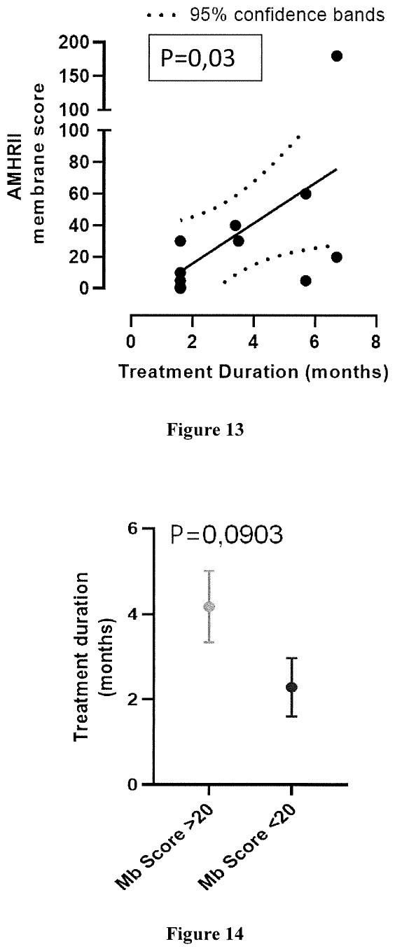

FIG. 13 illustrates a graph showing the correlation between (A) the duration of the treatment of patients with (i) the 3C23K anti-AMHRII antibody and (ii) Lonsurf comprising the combination of trifluridine and tipiracil and (B) the level of AMHRII expression as determined by the membranous score value by immuno-histo-chemistry. Abscissa: treatment duration, as expressed in months; Ordinate: level of AMHRII expression, as expressed as the membranous score value. Correlation analysis was performed according to the Spearman r test at 95% confidence interval.

FIG. 14 illustrates a graph showing (A) the duration of the treatment of patients with (i) the 3C23K anti-AMHRII antibody and (ii) Lonsurf comprising the combination of trifluridine and tipiracil as a function of (B) the level of AMHRII expression as determined by the membranous score value. Abscissa; left part, treated patients having an AMHRII membranous score >20; right part: treated patients having an AMHRII membranous score <20. Ordinate: treatment duration, as expressed in months. In FIG. 14, the comparison was performed between treatment durations of 4.2 months and 2.3 months, respectively.

### DETAILED DESCRIPTION OF THE INVENTION

The inventors have unexpectedly shown that the AMHRII, an AMH receptor, is expressed at the cell membrane of a plurality of a variety of non-gynecologic cancer tissues.

The term “AMHR-II” denotes the human Anti-Müllerian Hormone type II Receptor. The sequence of the human AMHR-II is described as SEQ ID NO. 18 herein (lacking the signal peptide MLGSLGLWALLPTAVEA (SEQ ID NO: 17)

As used herein, “non-gynecologic” cancers encompass any cancer that is not encompassed by the term “gynecologic” cancers.

As used herein, “gynecologic” cancers are selected in the group consisting of ovarian cancer, cervical cancer, endometrial cancer, gestational trophoblastic disease cancer (choriocarcinoma), uterine sarcoma, vaginal cancer, vulvar cancer and Fallopian tube cancer.

Then, as used herein, a “non-gynecologic” cancer consists of a cancer that does not consist of a cancer selected in the group consisting of ovarian cancer, cervical cancer, endometrial cancer, gestational trophoblastic disease cancer, uterine sarcoma, vaginal cancer, vulvar cancer and Fallopian tube cancer.

As used herein, the term “PDX” is an acronym for the expression “Patient-Derived Xenograft”. Patient-Derived Xenografts are highly used in vivo models of cancers, and especially in in vivo models of human cancers, where tissue or cells from a patient's tumor are implanted, i.e. “grafted”, into an immuno-deficient non-human mammal, e.g. an immuno-deficient mouse.

As it is shown in the examples herein, the inventors have found that AMHRII is expressed at the cell membrane of non-gynecologic cancer tissues with a variable frequency depending of the non-gynecologic cancer type which is considered. Illustratively, as shown in the examples herein, AMHRII is expressed more frequently by cancer cells derived from tumor tissue originating from patients affected with adrenocortical cancer than by cancer cells derived from tumor tissue originating from patients affected with a head and neck cancer. This means that these two types of cancers are eligible for an anti-cancer treatment targeting AMHRII, but that such an anti-cancer treatment will be less frequently relevant for treating patients affected with a head and neck cancer.

As it is shown in the examples herein, any non-gynecologic cancer, e.g. a liver cancer, a colorectal cancer or a kidney cancer, may be treated by an AMHRII-binding agent, provided that tumor cells from the said non-gynecologic tumor express AMHRII at their membrane, thus provided that the presence of AMHRII proteins at the tumor cell membrane can be detected or determined according to any method.

Thus, the experimental data provided in the examples herein show that the same AMHRII-binding agent, here an anti-AMHRII monoclonal antibody, is effective for treating a plurality of distinct kinds of cancer provided that the AMHRII target protein is expressed at the tumor cells membrane.

Incidentally, in the field of anti-cancer active ingredients consisting of target-binding molecules, e.g. target-binding antibodies, the situation wherein the same active ingredient is effective for treating a plurality of distinct cancers is not unprecedented. Illustratively, the anti-PD1 antibody named pembrolizumab has been authorized by the US Food and Drug Administration (FDA) as an active ingredient useful in the treatment of a variety of distinct kinds of cancers, provided that the said cancers share the same physiological features.

Thus, an individual affected with a non-gynecologic cancer may be treated for the said cancer with an AMHRII-binding agent as described herein when AMHRII membrane expression by the tumor cells previously collected from the said individual is detected or otherwise determined by an appropriate method.

In some embodiments, expression of AMHRII at the cell membrane of cancer cells encompasses that the said cancer cells express AMHRII at a given quantifiable level or higher than the said quantifiable level.

Thus, according to some embodiments, responsiveness of an individual affected with a non-gynecologic cancer to a treatment with an AMHRII-binding molecule may be assessed by determining whether non-gynecologic cancer cells from a sample previously collected from the said individual express AMHRII at their membrane.

According to some embodiments, responsiveness of an individual affected with a non-gynecologic cancer to a treatment with an AMHRII-binding molecule may be assessed by determining whether non-gynecologic cancer cells from a sample previously collected from the said individual express AMHRII at their membrane above a determined threshold value.

The AMHRII membrane expression level that may be used in some embodiments for determining the responsiveness of a patient affected with a non-gynecologic cancer to a treatment with a AMHRII-binding agent, e.g. an anti-AMHRII antibody, may be assessed with a variety of techniques, which include (i) the percentage of tumor cells contained in a tumor sample that express AMHRII at their membrane, (ii) the mean number of AMHRII proteins at the tumor cell membrane and (iii) the FACS AMHRII signal profile of the tumor cells contained in a tested tumor cell sample.

According to some embodiments, cancer cells comprised in a tumor sample previously collected for an individual affected with a non-gynecologic cancer may be assessed as expressing membranous AMHRII when membranous AMHRII is detected in 5% or more of the tumor cells comprised in the said tumor sample. The results shown in the examples herein allow expecting that the efficiency of a treatment of a non-gynecologic cancer with a AMHRII-binding agent, e.g. an anti-AMHRII antibody, correlates with the level of expression of membranous AMHRII.

Thus, in some embodiments, an individual affected with a non-gynecologic cancer is determined as being responsive to a treatment with an AMHRII-binding agent when 5% or more of the tumor cells comprised in a tumor sample previously collected from the said individual express AMHRII at their membrane.

Methods for determining the frequency (e.g. the percentage) of tumor cells expressing membrane AMHRII proteins are disclosed elsewhere in the present specification, including in the examples herein.

According to some embodiments, responsiveness of a patient affected with a non-gynecologic cancer to a cancer treatment with a AMHRII-binding agent, e.g. an anti-AMHRII antibody, may be assessed by determining the mean number of AMHRII proteins present at the membrane of the tumor cells contained in a tumor sample previously collected from the said patient.

In some embodiments, a patient affected with a non-gynecologic cancer may be classified as responsive to a treatment with a AMHRII-binding agent, e.g. responsive to a treatment with an anti-AMHRII antibody, when the mean number of membrane AMHRII proteins expressed by the tumor cells contained in a tumor sample previously collected from the said patient is of 10 000 AMHRII proteins or more.

Assessing the number of AMHRII proteins expressed at the tumor cell membrane may be performed by using conventional methods comprising (a) a step of incubating a sample containing the cells from a tumor tissue sample previously collected from the patient with a detectable compound that binds specifically with AMHRII protein, such as a fluorescently labeled anti-AMHRII antibody, and further (b) a step of determining the number of the said detectable compounds, e.g. the number of fluorescently labeled anti-AMHRII antibodies, bound to each tested cell from the said sample. Assessing the number of AMHRII proteins expressed at the tumor cell membrane may be, for instance, performed by using the well-known Fluorescence Activated Cell Sorting (FACS) technique, as it is shown in the examples herein.

In still other embodiments, a patient affected with a non-gynecologic cancer may be classified as responsive to a treatment with a AMHRII-binding agent, e.g. classified as responsive to a treatment with an anti-AMHRII antibody, by analysis of the AMHRII FACS profile of the tumor cells contained in a tumor sample previously collected from the said patient.

According to these still other embodiments, a patient affected with a non-gynecologic cancer may be classified as responsive to a treatment with a AMHRII-binding agent, e.g. classified as responsive to a treatment with an anti-AMHRII antibody when, in a method of fluorescence activated cell sorting (FACS), the ratio of (i) the mean fluorescence intensity of the tumor cells incubated with an anti-AMHRII fluorescently labeled antibody to (ii) the mean fluorescence intensity (MFI) value obtained from tumor cells incubated with an isotypic fluorescently labeled antibody is of 1.5 or more.

For determining the said mean fluorescence intensity ratio, both the isotypic antibody and the anti-AMHRII antibody are labeled with the same fluorescent agent, such as the Alexa Fluor 488 dye commercialized by the Company ThermoFisher Scientific, as shown in the examples herein.

In some further embodiments, responsiveness of a non-gynecologic cancer individual to a treatment with an AMHRII-binding agent may be determined by calculating an AMHRII expression score allowing to discriminate between (i) membrane AMHRII-expressing cancer cells derived from cancers that may be treated with an AMHRII-binding agent and (ii) membrane AMHRII-expressing cancer cells derived from cancers that may not be treated with an AMHRII-binding agent.

Thus, the inventors have determined that patients affected with a non-gynecologic cancer described herein, who are especially eligible to a cancer treatment with an AMHRII-binding agent described herein, i.e. who are especially responsive to a cancer treatment with an AMHRII-binding agent described herein, encompass those having cancer tumors expressing AMHRII at the cell membrane at a sufficiently high level for consisting in relevant cell targets to be destroyed.

Then, according to these further embodiments, the inventors have determined that a minimal AMHRII expression level measured in a cancer cell sample from a non-gynecologic cancer patient may confirm that the said patient is responsive to a treatment with a AMHRII-binding agent and that the said patient may thus be treated by an AMHRII-binding agent described herein.

Responsiveness of an individual affected with a non-gynecologic cancer to a treatment with an AMHRII-binding agent may thus also be determined when AMHRII expression level by cancer cells comprised in a sample previously collected from the said individual is assessed by both determining (i) the frequency of tumor cells expressing membranous AMHRII, e.g. the percentage of tumor cells expressing AMHRII at their membrane and (ii) the level of AMHRII membrane expression by the said tumor cells, e.g. the mean number of membranous AMHRII proteins per cell.

Thus, in some of these further embodiments, responsiveness of a patient affected with a non-gynecologic cancer to a human AMHRII-binding agent, e.g. to an anti-human AMHRII antibody, in a sample of tumor cells previously collected from the said patient, may be assessed by determining that (i) the tumor cells contained in the said sample exhibit a minimal mean number of human AMHRII proteins at their membrane and that (ii) the frequency of the cells expressing human AMHRII at their membrane, e.g. the percentage of cells expressing human AMHRII at their membrane, if of at least a threshold value.

Accordingly, it is also described herein a further method that may also be used for determining a specific AMHRII expression score value allowing to discriminate between (i) non-gynecologic cancer patients that are not eligible to a cancer treatment with an AMHRII-binding agent, i.e. non-gynecologic cancer patients that are not responsive to a cancer treatment with an AMHRII-binding agent and (ii) non-gynecologic cancer patients that are eligible to a cancer treatment with a AMHRII-binding agent, i.e. non-gynecologic cancer patients that are responsive to a cancer treatment with a AMHRII-binding agent.

More precisely, according to embodiments of the above method, patients affected with a non-gynecologic cancer described herein and who may be treated against cancer with an AMHRII-binding agent as described in the present specification may be preferably those for which an AMHRII expression score is of 1.0 or more has been determined, which includes those for which an AMHRII expression score is of 1.5 or more has been determined.

The membranous AMHRII expression score may be based on the immuno-histochemical evaluation of the AMHRII expression by the cancer cells tested, and wherein an individual membranous AMHRII score for a given cancer cell sample (i) is assigned as being 0 if no AMHRII expression is detectable, (ii) is assigned as being 1 if a significant AMHRII expression is detected and (iii) is assigned as being 2 if a high AMHRII expression is detected and (iv) is assigned as being 3 if an over-expression of AMHRII is detected.

Indeed, there is a relationship between (i) the score assigned to the membranous AMHRII expression level through the above-described immuno-histochemical evaluation and (ii) the mean number of AMHRII proteins expressed per cancer cell. It is shown in the examples herein that the membranous AMHRII expression level, allowing assigning an individual membranous AMHRII score, may also be assessed by determining the mean number of membranous AMHRII proteins per cell, starting from a sample of tumor cells that has been previously collected from a patient affected with a non-gynecologic cancer.

According to the above embodiments of determining responsiveness of an individual affected with a non-gynecologic cancer to a treatment with a AMHRII-binding agent, i.e. to a treatment with an anti-AMHRII antibody, a membranous AMHRII expression score is determined, for a given cancer cell sample, by taking into account both (i) the frequency of AMHRII-expressing cells in the said cancer cell sample and (ii) the level of AMHRII expression by the said AMHRII-expressing cells. Typically, an AMHRII expression score of a given cancer cell sample is determined by the following formula (I):

E-SCORE=FREQ×AMHRII_LEVEL, wherein
E-SCORE means the AMHRII expression score value for a given cancer cell sample,
FREQ means the frequency of the cells contained in the said cancer cell sample for which membrane AMHRII expression is detected, and
AMHRII_LEVEL means the level of expression of AMHRII by the AMHRII-expressing cells contained in the said given cancer cell sample.

Illustratively, a E-SCORE of 1.0 is determined for a given cancer cell sample wherein (i) 50% of the cells express AMHRII (FREQ value of 0.5) and (ii) the AMHRII expression level (AMHRII_LEVEL) is of 2.

In preferred embodiments, an AMHRII expression score (or E-SCORE) is determined by immunohistological methods as shown in the examples herein. According to these preferred embodiments, AMHRII membrane expression is assessed by using a detectable antibody specific for AMHRII and by (i) determining the frequency of cells having the said anti-AMHRII antibody bound thereto and (ii) determining the intensity of the signal generated by the said detectable anti-AMHRII antibody after its binding to the membrane-expressed AMHRII.

Although, as it is shown in the examples herein, AMHRII-expressing cancer cells having a AMHRII expression score of 1.5 or more have been determined for various cancers, albeit to distinct frequencies. Illustratively, the inventors have shown herein that cancer cells derived from colon tumors are classified as AMHRII positive (i.e. having a AMHRII score of 1.5 or more) with a higher frequency than cancer cells derived from head and neck cancer.

For determining the level of AMHRII membrane expression, detection of AMHRII at the cell membrane shall be most preferably performed by using an anti-AMHRII monoclonal antibody having a high affinity and high specificity for AMHRII, which is illustrated in the examples by the 3C23K anti-AMHRII monoclonal antibody.

Further, determination of AMHRII expression by an immuno-histochemical method with the view of determining a AMHRII score most preferably involves a careful pretreatment of the tissue sample before contacting the said sample with an appropriate detection reagent (e.g. a high affinity anti-AMHRII monoclonal antibody such as monoclonal 3C23K antibody, having a Kd value of 55.3 pM for binding to AMHRII). Sample pretreatment shall allow increasing the availability to the detection reagent of the AMHRII molecules expressed at the cell surface. Illustratively, as shown in the examples herein, staining method comprises an appropriate combination of specific steps such as (i) a high-temperature dewaxing by exposure to a microwave source and (ii) a system for amplifying the signal generated by the binding of an AMHRII-binding reagent, such as a biotinylated anti-AMHRII antibody that may be subsequently complexed with a streptavidin-conjugated detectable reagent. A pretreatment dewaxing step has appeared to be important for reversing the detection signal extinction effect due to the prior tissue fixation step. The inventors have shown that AMHRII detectability is particularly sensitive to the action of formalin which is used for the tissue fixation step.

In the context of the present invention, this means that an AMHRII-binding agent, such an anti-AMHRII antibody, will be a useful therapeutic agent with a higher frequency for treating patients affected with a colon cancer than for treating patients affected with a head and neck cancer. This also means that, although a AMHRII-binding agent may be a relevant therapeutic agent for treating patients affected with head and neck cancer, it will be preferred to test previously for the AMHRII expression of the tumor-derived cancer cells for deciding that a specific patient will be administered with a AMHRII binding agent as described herein.

Further, the inventors have shown that anti-AMHRII antibodies may be advantageously used for treating those non-gynecologic cancers.

Thus, the inventors have shown herein that pharmaceutical agents targeting AMHRII are useful as novel therapeutic tools for preventing or treating non-gynecologic cancers.

According to the invention, the expression “comprising”, such as in “comprising the steps of”, is also understood as “consisting of”, such as in “consisting of the steps of” is also understood as “consisting of”, such as “consisting of the steps of”.

The AMH receptor (AMHR or AMHR2 or AMHRII) is a serine/threonine kinase with a single transmembrane domain belonging to the family of type II receptors for TGF-beta-related proteins. Type II receptors bind the ligand on their own but require the presence of a type I receptor for signal transduction. Imbeaud et al. (1995, Nature Genet, Vol. 11: 382-388,) cloned the human AMH type II receptor gene. The human AMH receptor protein consists of 573 amino acids: 17, 127, 26, and 403 of the 573 amino acids form a signal sequence, extracellular domain (ECD), transmembrane domain, and intracellular domain containing a serine/threonine kinase domain, respectively

As used herein, the term “AMHRII” refers to the human Anti-Müllerian Hormone Type II Receptor having the amino acid sequence of SEQ ID NO. 17.

Expression of anti-Müllerian hormone receptor (AMHRII) was already described in the art in gynecologic cancers, tumors which are largely infiltrated by immune myeloid cells. AMHRII has been identified as a target molecule for treating gynecologic cancers. Antibodies directed to AMHRII have been produced as therapeutic tools for treating these cancers. It may be cited notably the 12G4 anti-AMHRII antibody and variants thereof described in the PCT applications no WO 2008/053330 and no WO 2011/141653 for treating ovarian cancers, as well as the 3C23K anti-AMHRII antibody described in the PCT application. It may also be mentioned the PCT application no WO 2017/025458 which disclosed a specific treatment strategy against ovarian cancer by using anti-AMHRII antibody drug conjugates.

The inventors have now unexpectedly found that AMHRII was expressed at the surface of various human cancer cells, which include colon cancer, liver cancer, hepatocellular carcinoma, testis cancer, thyroid cancer, gastric cancer, gastrointestinal cancer, bladder cancer, pancreatic cancer, head and neck cancer, kidney cancer, liposarcoma, fibrosarcoma, pleuramesothelioma, melanoma, sarcoma, brain cancer, osteocarcinoma, breast cancer, prostate cancer and leukemia. The inventors have also found that there is no relationship between (i) the AMHRII gene expression by cancer cells and (ii) the cell membrane AMHRII protein expression by the same cancer cells.

The inventors' findings regarding AMHRII surface expression by human cancer cells notably derive from immunohistochemical assays with an anti-AMHRII antibody that were performed by using human solid tumor tissue samples previously obtained from cancer patients. The inventors' findings relating to AMHRII surface expression by human cancer cells were also obtained from immunohistochemical assays with an anti-AMHRII antibody that were performed on tumor tissue samples originating from human primary cancer cells xenografts in mice.

The present inventors have also shown that anti-AMHRII antibodies are useful for treating non-gynecologic human cancers that express AMHRII at the tumor cell surface, and especially those AMHRII-expressing cancers disclosed in the present specification. Notably, good anti-cancer activity has been shown by immunoconjugates comprising anti-AMHRII antibodies conjugated to a cytotoxic molecule.

The inventors have shown that an anti-AMHRII antibody that had proved anti-tumor efficacy against AMHRII-expressing gynecologic cancers in the art is also useful for preventing or treating non-gynecologic AMHRII-expressing cancers, and especially those AMHRII-expressing cancers disclosed in the present specification.

More precisely, it is shown in the examples herein that the anti-AMHRII antibody named 3C23K exerts an anti-tumor activity in vivo against human liver cancer. Importantly, the in vivo anti-tumor activity of the anti-AMHRII 3C23K antibody against human liver cancer is of the same order of magnitude as sorafenib, which is a well-known anticancer agent for treating liver cancers and especially hepatocellular carcinoma.

Still further, the examples herein have also shown that the anti-AMHRII 3C23K antibody induces no detectable toxic event in vivo, whereas a treatment with sorafenib in the same in vivo conditions caused a significant body weight loss.

Yet further, as disclosed herein, a toxic immunoconjugate derivative of the anti-AMHRII 3C23K antibody (ADC for Antibody Drug Conjugate) exerts a good anti-cancer activity against cancers that express the AMHRII protein at the cell surface.

Thus, the present invention relates to a human AMHRII-binding agent for its use for preventing or treating a cancer selected in a group of cancers comprising colon cancer, liver cancer, hepatocellular carcinoma, testis cancer, thyroid cancer, gastric cancer, gastrointestinal cancer, bladder cancer, pancreatic cancer, head and neck cancer, kidney cancer, liposarcoma, fibrosarcoma, pleuramesothelioma, melanoma, sarcoma, brain cancer, osteocarcinoma, breast cancer, prostate cancer and leukemia.

This invention also concerns the use of a human AMHRII-binding agent for the preparation of a medicament for preventing or treating a cancer selected in a group of cancers comprising colon cancer, liver cancer, hepatocellular carcinoma, testis cancer, thyroid cancer, gastric cancer, gastrointestinal cancer, bladder cancer, pancreatic cancer, head and neck cancer, kidney cancer, liposarcoma, fibrosarcoma, pleuramesothelioma, melanoma, sarcoma, brain cancer, osteocarcinoma, breast cancer, prostate cancer and leukemia.

This invention also pertains to a method for preventing or treating a cancer selected in a group of cancers comprising colon cancer, liver cancer, hepatocellular carcinoma, testis cancer, thyroid cancer, gastric cancer, gastrointestinal cancer, bladder cancer, pancreatic cancer, head and neck cancer, kidney cancer, liposarcoma, fibrosarcoma, pleuramesothelioma, melanoma, sarcoma, brain cancer, osteocarcinoma, breast cancer, prostate cancer and leukemia, wherein the said method comprises a step of administering to an individual in need thereof an AMHRII-biding agent as disclosed in the present specification.

An AMHRII-binding agent that may be used according to the present invention does not require a mimicking of the MIS natural ligand activity. Thus, there is no need that an AMHRII-binding agent that may be used according to the invention activates any cell signaling pathway upon its binding to AMHRII. Instead, sole the ability of the said agent to bind to AMHRII is required, since the said agent is used exclusively for targeting a cytotoxicity-inducing activity, such as a cytotoxicity-inducing entity, which encompasses an anti-AMHRII cytotoxic immuno-conjugate, an ADCC-inducing or an ADC-inducing anti-AMHRII antibody or a CAR T-cell expressing an AMHRII-binding engineered receptor.

### AMHRII Binding Agent

As used herein, an AMHRII-binding agent encompasses any agent that specifically binds to AMHRII and which, when presented in an appropriate manner, will cause the death of the target cells expressing AMHRII at their surface after that the said agent has bound the cell membrane-expressed AMHRII.

An AMHRII-binding agent that is used for treating a cancer as described herein may also be termed a “therapeutic AMHRII-binding agent” herein.

Generally, a AMHRII-binding agent encompasses a protein or a nucleic acid that specifically binds to AMHRII.

AMHRII-binding proteins mainly encompass proteins comprising one or more Complementary Determining Regions (CDRs) that originate from an anti-AMHRII antibody or from an AMHRII-binding fragment of an anti-AMHRII antibody, it being understood that the said AMHRII-binding proteins may be expressed as Chimeric Antigen Receptors (CARs) by engineered cells such as CAR-T-cells. CAR NK T-cells or CAR Macrophages.

AMHRII-binding nucleic acids mainly encompass nucleic acid aptamers that have been especially selected for their specific binding properties to AMHRII.

In some preferred embodiments, the AMHRII-binding agent is an anti-AMHRII antibody or an AMHRII-binding fragment thereof.

In most preferred embodiments, the AMHRII-binding agent is an anti-AMHRII monoclonal antibody or an AMHRII-binding fragment thereof.

According to these preferred embodiments, anti-AMHRII monoclonal antibodies encompass chimeric anti-AMHRII antibodies, humanized anti-AMHRII antibodies and human AMHRII antibodies, as well as the AMHRII-binding fragments and AMHRII-binding derivatives thereof.

Various AMHRII antibodies are known in the art and may be used according to the invention as AMHRII-binding agents. For the purpose of performing the present invention, the one skilled in the art may use, for illustration, the recombinant human anti-AMHRII marketed by Creative Biolabs under the reference no MHH-57.

In some embodiments, an anti-AMHRII antibody that may be used according to the invention is the humanized 12G4 antibody disclosed in the PCT application no WO 2008/053330.

In some other embodiments, the said anti-AMHRII antibodies are the humanized antibodies described in the PCT application no WO 2011/141653, which humanized antibodies encompass the 3C23 antibodies as well as the variants thereof, which variants thereof include the 3C23K humanized antibody.

In still further embodiments, the said anti-AMHRII antibodies are those described in the PCT application no WO 2017/025458. According to these further embodiments, the PCT application no WO 2017/025458 disclosed AMHRII-binding agents under the form of Antibody Druc Conjugates (ADC) wherein the said anti-AMHRII antibodies are linked to a cytotoxic agent.

A monoclonal antibody against Mullerian Hormone type II receptor (and humanized derivatives thereof) has been developed in the art for the treatment of ovarian cancer (see EP 2097453B1 and U.S. Pat. No. 8,278,423, which is hereby incorporated by reference in its entirety).

Among the AMHRII-binding agents that may be used according to the invention, the one skilled in the art may use the monoclonal antibody 12G4 (mAb 12G4), or chimeric or humanized variants thereof, including such an antibody which has been derivatized with a drug or detectable label to form an ADC. The hybridoma producing mAbl2G4 has been deposited at the Collection Nationale de Cultures de Microorganismes (CNCM, Institut Pasteur, 25 rue du Docteur Roux, 75724 Paris Cedex 15, France), in accordance with the terms of Budapest Treaty, on the 26 of Sep. 2006) and has CNCM deposit number 1-3673. The variable domain of the light and heavy chains of the mAb 12G4 have been sequenced as have been the complementarity determining regions (CDRs) of mAb 12G4 (see EP 2097453B1 and U.S. Pat. No. 8,278,423, which is hereby incorporated by reference in its entirety). mAb 12G4 and its chimeric or humanized variants can be used for the production of ADC as disclosed herein.

The PCT application no PCT/FR2011/050745 (International Publication no WO/2011/141653) and U.S. Pat. No. 9,012,607, each of which is hereby incorporated by reference in its entirety, disclose novel humanized antibodies that are derived from the murine 12G4 antibody. These humanized antibodies may be used as AMHRII-binding agents for the purpose of the present invention. In particular embodiments disclosed in the PCT application no WO/2011/141653, the antibodies are those identified as the 3C23 and 3C23K. The nucleic acid sequences and polypeptide sequences of these antibodies are provided as SEQ ID NOs: 1-16 herein. In some aspects of the invention, the anti-AMHRII antibodies of interest may be referred to as “comprising a light chain comprising SEQ ID NO: and a heavy chain comprising SEQ ID NO:”. Thus, in various embodiments, particularly preferred antibodies, including for the generation of ADC, comprise:
a) a light chain comprising SEQ ID NO: 2 and a heavy chain comprising SEQ ID NO: 4 (3C23 VL and VH sequences without leaders);
b) a light chain comprising SEQ ID NO: 6 and a heavy chain comprising SEQ ID NO: 8 (3C23K VL and VH sequences without leaders);
c) a light chain comprising SEQ ID NO: 10 and a heavy chain comprising SEQ ID NO: 12 (3C23 light and heavy chains without leaders);
d) a light chain comprising SEQ ID NO: 14 and a heavy chain comprising SEQ ID NO: 16 (3C23K light and heavy chains without leaders).

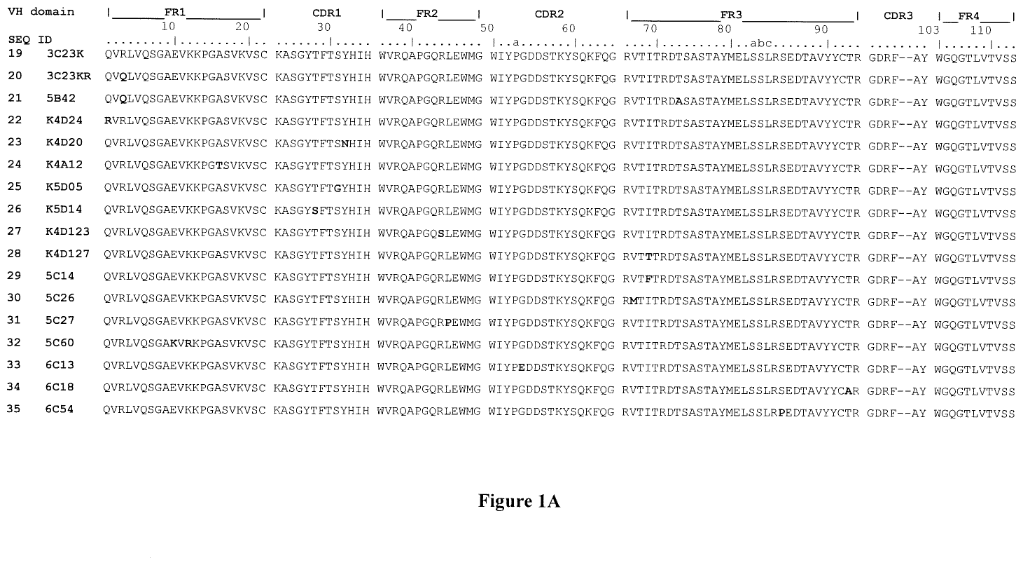

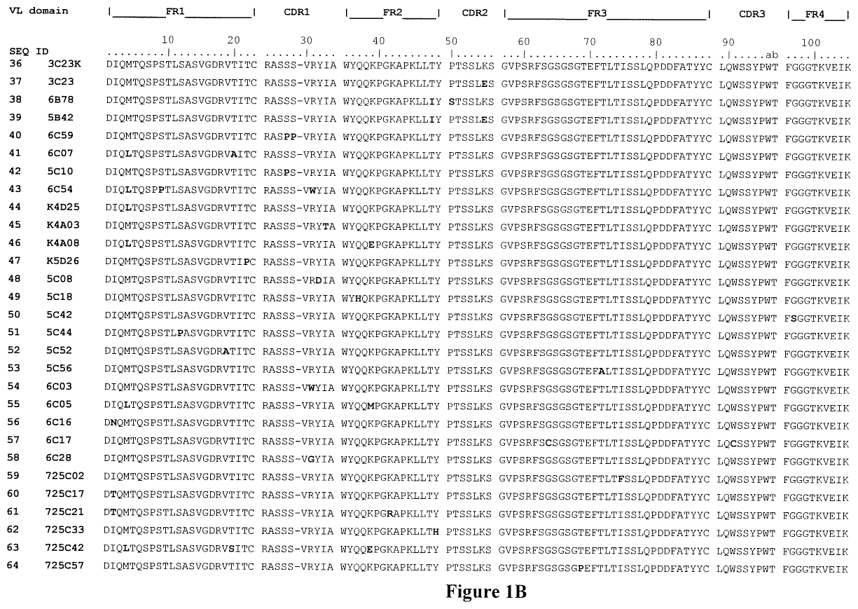

Other antibodies (e.g., humanized or chimeric antibodies) can be based upon the heavy and light chain sequences provided in FIGS. 1A and 1B (e.g., antibodies, such as humanized or chimeric antibodies containing the CDR sequences disclosed within the Figures) can be used as anti-MAHRII-binding agents of interest, including for the formation of ADCs. Thus, the invention also pertains to the use of anti-AMHRII antibodies comprising/containing CDRs comprising (or consisting of) the following sequences:

CDRL-1: RASX1X2VX3X4X5A (SEQ ID NO. 65), where X1
and X2 are, independently, S or P, X3 is R or W
or G, X4 is T or D, and X5 is I or T;
CDRL-2 is PTSSLX6S (SEQ ID NO. 66) where X6 is K
or E; and
CDRL-3 is LQWSSYPWT (SEQ ID NO. 67);
CDRH-1 is KASGYX7FTX8X9HIH (SEQ ID NO. 68) where X7
is S or T, X8 is S or G and X9 is Y or N;
CDRH-2 is WIYPX10DDSTKYSQKFQG (SEQ ID NO. 69)
where X10 is G or E and
CDRH-3 is GDRFAY (SEQ ID NO. 70).

This invention also relates to the use of ADCs generated using such anti-AMHRII antibodies for treating the non-gynecologic cancers that are specified herein.

Antibodies (e.g., chimeric or humanized) within the scope of this application include those disclosed in the following table: Alternatively, human monoclonal antibodies that specifically bind to AMHR-II can be used for the preparation of ADCs. 3C23K antibody is defined by:
SEQ ID NO: 19 for VH amino acid sequence
SEQ ID NO: 36 for VL amino acid sequence

Table 1 hereunder lists anti-AMHRII humanized antibodies that may be used according to the invention.

TABLE 1
anti-AMHRII antibodies

| | Mutations | | | |
|---|---|---|---|---|
| | | SEQ ID in | | SEQ ID in |
| | | sequence | | sequence |
| Antibody | VH mutations | listing | VL mutations | listing |
| 3C23K | | 19 | | 36 |
| 3C23 | | 19 | L-K55E | 37 |
| 3C23KR | H-R3Q | 20 | | 36 |
| 6B78 | H-R3Q | 20 | L-T48I, L-P50S | 38 |
| 5B42 | H-R3Q, H-T73A | 21 | L-T48I, L-K55E | 39 |
| K4D-24 | H-Q1R | 22 | | 36 |
| 6C59 | H-Q1R | 22 | L-S27P, L-S28P | 40 |
| K4D-20 | H-Y32N | 23 | | 36 |
| K4A-12 | H-A16T | 24 | | 36 |
| K5D-05 | H-S31G | 25 | | 36 |
| K5D-14 | H-T28S | 26 | | 36 |
| K4D-123 | H-R44S | 27 | | 36 |
| K4D-127 | H-I69T | 28 | | 36 |
| 6C07 | H-I69T | 28 | L-M4L, L-T20A | 41 |
| 5C14 | H-I69F | 29 | | 36 |
| 5C26 | H-V67M | 30 | L-S27P | 42 |
| 5C27 | H-L45P | 31 | | 36 |
| 5C60 | H-E10K, H-K12R | 32 | | 36 |
| 6C13 | H-G53E | 33 | | 36 |
| 6C18 | H-T93A | 34 | | 36 |
| 6C54 | H-S84P | 35 | L-M4L, L-S9P, L- | 43 |
| | | | R31W | |
| K4D-25 | | 19 | L-M4L | 44 |
| K4A-03 | | 19 | L-I33T | 45 |
| K4A-08 | | 19 | L-M4L, L-K39E | 46 |
| K5D-26 | | 19 | L-T22P | 47 |
| 5C08 | | 19 | L-Y32D | 48 |
| 5C10 | | 19 | L-S27P | 42 |
| 5C18 | | 19 | L-Q37H | 49 |
| 5C42 | | 19 | L-G97S | 50 |
| 5C44 | | 19 | L-S12P | 51 |
| 5C52 | | 19 | L-19A | 52 |
| 5C56 | | 19 | L-T72A | 53 |
| 6C03 | | 19 | L-R31W | 54 |
| 6C05 | | 19 | L-M4L, L-M39K | 55 |
| 6C16 | | 19 | L-I2N | 56 |
| 6C17 | | 19 | L-G63C, L-W91C | 57 |
| 6C28 | | 19 | L-R31G | 58 |
| 725C02 | | 19 | L-I75F | 59 |
| 725C17 | | 19 | L-I2T | 60 |
| 725C21 | | 19 | L-I2T, L-K42R | 61 |
| 725C33 | | 19 | L-Y49H | 62 |
| 725C42 | | 19 | L-M4L, L-T20S, | 63 |
| | | | L-K39E | |
| 725C44 | | 19 | L-S27P | 42 |
| 725C57 | | 19 | L-T69P | 64 |

### Anti-AMHRII Antibodies, AMHRII-Binding Fragments or AMHRII-Binding Derivatives of Anti-AMHRII Antibodies

The term “antibody” is used in the broadest sense and includes monoclonal antibodies (including full length or intact monoclonal antibodies), polyclonal antibodies, multivalent antibodies, multispecific antibodies (e.g., bispecific antibodies), and antibody fragments (see below) so long as they exhibit the desired biological activity.

Thus, as used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. Unless specifically noted otherwise, the term “antibody” includes intact immunoglobulins and “antibody fragments” or “antigen binding fragments” that specifically bind to AMHRII to the substantial exclusion of binding to other molecules (i.e. molecules unrelated to AMHRII). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, 111.); Kuby, J., Immunology, 7^{th} Ed., W.H. Freeman & Co., New York, 2013.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The modifier “monoclonal” is not to be construed as requiring production of the antibody by any particular method. For example, the monoclonal antibodies to be used in accordance with the invention may be made by the hybridoma method first described by Kohler et al, Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al, Nature 352:624-628 (1991) or Marks et al, J. Mol Biol. 222:581-597 (1991), for example.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations, κ and λ light chains refer to the two major antibody light chain isotypes.

As used herein the term “complementarity determining region” or “CDR” refers to the part of the two variable chains of antibodies (heavy and light chains) that recognize and bind to the particular antigen. The CDRs are the most variable portion of the variable chains and provide the antibody with its specificity. There are three CDRs on each of the variable heavy (VH) and variable light (VL) chains and thus there are a total of six CDRs per antibody molecule. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VHCDR3 is located in the variable domain of the heavy chain of the antibody in which it is found, whereas a VLCDR1 is the CDR1 from the variable domain of the light chain of the antibody in which it is found. An antibody that binds LHR will have a specific VH region and the VL region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

“Framework regions” (hereinafter FR) are those variable domain residues other than the CDR residues. Each variable domain typically has four FRs identified as FR1, FR2, FR3 and FR4. If the CDRs are defined according to Kabat, the light chain FR residues are positioned at about residues 1-23 (LCFR1), 35-49 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues.

“Single-chain Fv” or “scFv” antibody fragments comprise the VH and VL domains of antibody, wherein these domains are present in a single polypeptide chain. Generally the Fv polypeptide further comprises a polypeptide linker between the VH and VL domains, which enables the scFv to form the desired structure for antigen binding. For a review of scFv, see Pluckthun in The Pharmacology of Monoclonal Antibodies, Vol 113, Rosenburg and Moore eds. Springer-Verlag, New York, pp. 269-315 (1994).

The term “diabodies” refers to small antibody fragments with two antigen-binding sites, which fragments comprise a heavy chain variable domain (VH) connected to a light chain variable domain (VL) in the same polypeptide chain (VH and VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al, Proc. Natl. Acad. Sci. USA, 90:6444-6448 (1993).

Diabodies or bi-specific antibodies can be roughly divided into two categories: immunoglobulin G (IgG)-like molecules and non-IgG-like molecules. IgG-like bsAbs retain Fc-mediated effector functions such as antibody-dependent cell-mediated cytotoxicity (ADCC), complement-dependent cytotoxicity (CDC), and antibody-dependent cellular phagocytosis (ADCP) (Spiess et al., 2015, Mol Immunol., Vol. 67(2): 95-106.). The Fc region of bsAbs facilitates purification and improves solubility and stability. Bi-specific antobodies in IgG-like formats usually have longer serum half-lives owing to their larger size and FcRn-mediated recycling (Kontermann et al., 2015, Bispecific antibodies. Drug Discov Today Vol. 20(7): 838-47). Non-IgG-like bsAbs are smaller in size, leading to enhanced tissue penetration (Kontermann et al., 2015, Bispecific antibodies. Drug Discov Today Vol. 20(7): 838-47).

According to some preferred embodiments, bispecific antibodies according to the invention comprise (i) a first antigen binding site that binds to AMHRII and (ii) a second antigen binding site that binds to a target antigen which is distinct from AMHRII and especially a target antigen that may be expressed by cancer cells or immune cells of the tumor microenvironment such as T-cells, NK or macrophages. In some embodiments, in such bispecific antibodies, the said second antigen binding site binds to a target antigen which is CD3 and allows the engagement of T-cells. This target antigen can also be PDL1 to unlock T-cells or CD16 to activate NK or macrophages.

The monoclonal antibodies specified herein specifically include “chimeric” anti-AMHRII antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

The monoclonal antibodies specified herein also encompass humanized anti-AMHRII antibodies. “Humanized” forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al, Nature 321:522-525 (1986); Riechmann et al., Nature 332:323-329 (1988); and Presta, Curr. Op. Struct. Biol. 2:593-596 (1992).

The monoclonal anti-AMHRII antibodies specified herein further encompass anti-AMHRII human antibodies. A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art. In one embodiment, the human antibody is selected from a phage library, where that phage library expresses human antibodies (Vaughan et al. Nature Biotechnology 14:309-314 (1996): Sheets et al. Proc. Natl. Acad. Sci. 95:6157-6162 (1998)); Hoogenboom and Winter, J. Mol. Biol, 227:381 (1991); Marks et al., J. Mol. Biol, 222:581 (1991)). Human antibodies can also be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368:812-13 (1994); Fishwild et al., Nature Biotechnology 14: 845-51 (1996); Neuberger, Nature Biotechnology 14: 826 (1996); Lonberg and Huszar, Intern. Rev. Immunol. 13:65-93 (1995). Alternatively, the human antibody may be prepared via immortalization of human B lymphocytes producing an antibody directed against a target antigen (such B lymphocytes may be recovered from an individual or may have been immunized in vitro). See, e.g., Cole et al, Monoclonal Antibodies and Cancer Therapy, Alan R. Liss, p. 77 (1985); Boerner et al., J. Immunol, 147 (1):86-95 (1991); and U.S. Pat. No. 5,750,373.

As used herein, “antibody mutant” or “antibody variant” refers to an amino acid sequence variant of the species-dependent antibody wherein one or more of the amino acid residues of the species-dependent antibody have been modified. Such mutants necessarily have less than 100% sequence identity or similarity with the species-dependent antibody. In one embodiment, the antibody mutant will have an amino acid sequence having at least 75% amino acid sequence identity or similarity with the amino acid sequence of either the heavy or light chain variable domain of the species-dependent antibody, more preferably at least 80%, more preferably at least 85%, more preferably at least 90%, and most preferably at least 95%. Identity or similarity with respect to this sequence is defined herein as the percentage of amino acid residues in the candidate sequence that are identical (i.e same residue) or similar (i.e. amino acid residue from the same group based on common side-chain properties, see below) with the species-dependent antibody residues, after aligning the sequences and introducing gaps, if necessary, to achieve the maximum percent sequence identity. None of N-terminal, C-terminal, or internal extensions, deletions, or insertions into the antibody sequence outside of the variable domain shall be construed as affecting sequence identity or similarity.

Humanized antibodies may be produced by obtaining nucleic acid sequences encoding CDR domains and constructing a humanized antibody according to techniques known in the art. Methods for producing humanized antibodies based on conventional recombinant DNA and gene transfection techniques are well known in the art (See, e.g., Riechmann L. et al. 1988; Neuberger M S. et al. 1985). Antibodies can be humanized using a variety of techniques known in the art including, for example, CDR-grafting (EP 239,400; PCT publication WO91/09967; U.S. Pat. Nos. 5,225,539; 5,530,101; and 5,585,089), veneering or resurfacing (EP 592,106; EP 519,596; Padlan E A (1991); Studnicka G M et al. (1994); Roguska M A. et al. (1994)), and chain shuffling (U.S. Pat. No. 5,565,332). The general recombinant DNA technology for preparation of such antibodies is also known (see European Patent Application EP 125023 and International Patent Application WO 96/02576).

It may be desirable to modify an anti-AMHRII antibody specified herein with respect to effector function, e.g. so as to enhance antigen-dependent cell-mediated cyotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al, J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al. Anti-Cancer Drug Design 3:219-230 (1989). WO00/42072 (Presta, L.) describes antibodies with improved ADCC function in the presence of human effector cells, where the antibodies comprise amino acid substitutions in the Fc region thereof. Preferably, the antibody with improved ADCC comprises substitutions at positions 298, 333, and/or 334 of the Fc region (Eu numbering of residues). Preferably the altered Fc region is a human IgGI Fc region comprising or consisting of substitutions at one, two or three of these positions. Such substitutions are optionally combined with substitution(s) which increase CIq binding and/or CDC.

Antibodies with altered CIq binding and/or complement dependent cytotoxicity (CDC) are described in WO99/51642, U.S. Pat. No. 6,194,551B1, U.S. Pat. No. 6,242,195B1, U.S. Pat. No. 6,528,624B1 and U.S. Pat. No. 6,538,124 (Idusogie et al). The antibodies comprise an amino acid substitution at one or more of amino acid positions 270, 322, 326, 327, 329, 313, 333 and/or 334 of the Fc region thereof (Eu numbering of residues).

In some embodiments, AMHRII-binding agents encompass glyco-engineered anti-AMHRII antibodies.

As used herein, the term “glycoengineering” refers to any art-recognized method for altering the glycoform profile of a binding protein composition. Such methods include expressing a binding protein composition in a genetically engineered host cell (e.g., a CHO cell) that has been genetically engineered to express a heterologous glycosyltransferase or glycosidase. In other embodiments, the glycoengineering methods comprise culturing a host cell under conditions that bias for particular glycoform profiles.

As used herein, a “glyco-engineered antibody” encompasses (i) an antibody comprising a hyper-galactosylated Fc fragment, (ii) an antibody comprising a hypo mannosylated Fc fragment, which encompasses a amannosylated Fc fragment, and (iii) an antibody comprising a hypo fucosylated Fc fragment, which encompasses a afucosylated Fc fragment. As used herein, a glyco-engineered fragment encompasses a Fc fragment having an altered glycosylation which is selected in a group comprising one or more of the following altered glycosylation (i) hyper-galactosylation, (ii) hypo-mannosylation and (iii) hypo-fucosylation. Consequently, a glyco-engineered Fc fragment from an anti-AMHRII antibody as used according to the invention encompass the illustrative examples of a hyper-galactosylated, a hypo-mannosylated and a hypo-fucosylated Fc fragment.

The one skilled in the art may refer to well-known techniques for obtaining anti-AMHRII antibodies comprising hyper-galactosylated Fc fragments, hypo mannosylated Fc fragments and hypo fucosylated Fc fragments that are known to bind to Fc receptors with a higher affinity than non-modified Fc fragments.

Glyco-engineered anti-AMHRII antibodies encompass anti-AMHRII antibodies comprising a hypofucosylated Fc fragment, which may also be termed a “low fucose” Fc fragment.

### Immunoconjugates, Especially Antibody Drug Conjugates (ADC)

AMHRII-binding agents that may be used for the purpose of the present invention encompass antibodies specified herein that are conjugated to a cytotoxic agent such as a chemotherapeutic agent, toxin (e.g. an enzymatically active toxin of bacterial, fungal, plant or animal origin, or fragments thereof), or a radioactive isotope (i.e., a radio conjugate). Such antibody conjugates encompass those described in the PCT application no WO 2017/025458. The PCT application no WO 2017/025458 notably disclosed the anti-AMHRII 3C23K antibody, as well as 3C23K ADC conjugates, for which in vivo anti-cancer activity is shown herein against non-gynecologic human cancers.

Cytotoxic agents encompass enzymatically active toxins. Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes.

A variety of radionuclides are available for the production of radioconjugate antibodies.

Conjugates of the antibody and cytotoxic agent are made using a variety of bifunctional protein coupling agents such as those dicslosed in the PCT application no WO 2017/025458.

Preferred immunoconjugates of anti-AMHRII ADC antibody conjugates are those described in the PCT application no WO 2017/025458

### CAR Cells, Including CAR T-Cells, CAR NK Cells and CAR Macrophages

In some embodiments, the human-AMHRII-binding agent is an AMHRII-binding receptor or an AMHRII-binding receptor-expressing cell, and especially an AMHRII-binding receptor-expressing CAR T-cell, an AMHRII-binding receptor CAR NK cell or an AMHRII-binding receptor-expressing CAR Macrophage.

Thus, in some embodiments, the human AMHRII-binding agent is an AMHRII-binding engineered receptor, and most preferably an AMHRII-binding engineered receptor for which the AMHRII-binding region thereof derives from a monoclonal anti-AMHRII antibody disclosed in the present specification.

Typically, the AMHRII-binding engineered receptor consists of a Chimeric Antigen Receptor (CAR) comprising (i) an extracellular domain, (ii) a transmembrane domain and (iii) an intracellular domain, and wherein the extracellular domain is an AMHRII-binding moiety which derives from an anti-AMHRII monoclonal antibody disclosed in the present specification. In some embodiments, the extracellular domain of the said AMHRII-binding engineered receptor comprises (i) an antibody VH chain comprising the CDRs derived from an anti-AMHRII monoclonal antibody disclosed herein and (ii) an antibody VL chain comprising the CDRs derived from an anti-AMHRII monoclonal antibody disclosed herein. In some embodiments, the extracellular domain of the said AMHRII-binding engineered receptor comprises the VH chain and the VL chain of an anti-AMHRII monoclonal antibody disclosed herein. In some embodiments, the extracellular domain of the said AMHRII-binding engineered receptor is a ScFv comprising the CDRs derived from the VH chain and the CH chain from an anti-AMHRII monoclonal antibody disclosed in the present specification, respectively. In some embodiments, the extracellular domain of the said AMHRII-binding engineered receptor is a ScFv comprising the VH chain and the CH chain from an anti-AMHRII monoclonal antibody disclosed in the present specification, respectively.

Is also encompassed herein an AMHRII-binding agent consisting of a cell expressing such an AMHRII-binding receptor, and especially a CAR T-cell, a CAR NK cell or a CAR Macrophage expressing such an AMHRII-binding receptor.

The term “chimeric antigen receptor” (CAR), as used herein, refers to a fused protein comprising an extracellular domain capable of binding to an antigen, a transmembrane domain derived from a polypeptide different from a polypeptide from which the extracellular domain is derived, and at least one intracellular domain. The “chimeric antigen receptor (CAR)” is sometimes called a “chimeric receptor”, a “T-body”, or a “chimeric immune receptor (CIR).” The “extracellular domain capable of binding to AMHRII” means any oligopeptide or polypeptide that can bind to AMHRII. The “intracellular domain” means any oligopeptide or polypeptide known to function as a domain that transmits a signal to cause activation or inhibition of a biological process in a cell. The “transmembrane domain” means any oligopeptide or polypeptide known to span the cell membrane and that can function to link the extracellular and signaling domains. A chimeric antigen receptor may optionally comprise a “hinge domain” which serves as a linker between the extracellular and transmembrane domains.

CAR T-cells are genetically engineered autologous T-cells in which single chain antibody fragments (scFv) or ligands are attached to the T-cell signaling domain capable of facilitating T-cell activation (Maher, J. (2012) ISRN Oncol. 2012:278093; Curran, K. J. et al. (2012) J. Gene Med. 14:405-415; Fedorov, V. D. et al. (2014) Cancer J. 20:160-165; Barrett, D. M. et al. (2014) Annu. Rev. Med. 65:333-347).

By “intracellular signaling domain” is meant the portion of the CAR that is found or is engineered to be found inside the T cell. The “intracellular signaling domain” may or may not also contain a “transmembrane domain” which anchors the CAR in the plasma membrane of a T cell. In one embodiment, the “transmembrane domain” and the “intracellular signaling domain” are derived from the same protein (e.g. CD3) in other embodiments; the intracellular signaling domain and the transmembrane domain are derived from different proteins (e.g. the transmembrane domain of a CD3 and intracellular signaling domain of a CD28 molecule, or vice versa).

By “co-stimulatory endodomain” is meant an intracellular signaling domain or fragment thereof that is derived from a T cell costimulatory molecule. A non-limiting list of T cell costimulatory molecules include CD3, CD28, OX-40, 4-1BB, CD27, CD270, CD30 and ICOS. The co-stimulatory endodomain may or may not include a transmembrane domain from the same or different co-stimulatory endodomain.

By “extracellular antigen binding domain” is meant the portion of the CAR that specifically recognizes and binds to AMHRII.

In preferred embodiments, the “extracellular binding domain” is derived from an anti-AMHRII monoclonal antibody. For example, the “extracellular binding domain” may include all or part of a Fab domain from a monoclonal antibody. In certain embodiments, the “extracellular binding domain” includes the complementarity determining regions of a particular anti-AMHRII monoclonal antibody. In still another embodiment, the “extracellular binding domain” is a single-chain variable fragment (scFv) obtained from an anti-AMHRII monoclonal antibody specified herein.

In preferred embodiments, the extracellular binding domain is derived from any one of the anti-AMHRII monoclonal antibodies described in the present specification and especially from the 3C23K anti-AMHRII monoclonal antibody.

### I. Extracellular Antigen Binding Domain

In one embodiment, the CAR of the current invention comprises an extracellular antigen binding domain from one of the anti-AMHRII monoclonal antibodies described herein.

In one embodiment, the extracellular binding domain comprises the following CDR sequences:

CDRL-1: RASX1X2VX3X4X5A (SEQ ID NO. 65), where X1
and X2 are, independently, S or P, X3 is R or W
or G, X4 is T or D, and X5 is I or T;
CDRL-2 is PTSSLX6S (SEQ ID NO. 66) where X6
is K or E; and
CDRL-3 is LQWSSYPWT (SEQ ID NO. 67);
CDRH-1 is KASGYX7FTX8X9HIH (SEQ ID NO. 68) where
X7 is S or T, X8 is S or G and X9 is Y or N;
CDRH-2 is WIYPX10DDSTKYSQKFQG (SEQ ID NO. 69)
where X10 is G or E and
CDRH-3 is GDRFAY (SEQ ID NO. 70)

II. Linker Between VL and VH Domains of KappaMab scFv

In a further embodiment, the anti-AMHRII VL is linked to the anti-AMHRII VH via a flexible linker. Specifically, the flexible linker is a glycine/serine linker of about 10-30 amino acids (for example 30, 25, 20, 19, 18, 17, 16, 15, 14, 13, 12, 11, 10, 9, 8, 7, 6, or 5 amino acids) and comprises the structure (Gly4Ser)³.

### III. Spacers Between Extracellular Antigen Binding Domain and Intracellular Signaling Domain

The extracellular antigen binding domain is linked to the intracellular signaling domain by the use of a “spacer”. The spacer is designed to be flexible enough to allow for orientation of the antigen binding domain in such a way as facilitates antigen recognition and binding. The spacer may derive from the anti-AMHRII immunoglobulins themselves and can include the IgGI hinge region or the CH2 and/or CH3 region of an IgG.

### IV. Intracellular Signaling Domain

The intracellular signaling domain comprises all or part of the CD3 chain. CD, also known as CD247, together with either the CD4 or CD8 T cell co-receptor is responsible for coupling extracellular antigen recognition to intracellular signaling cascades.

In addition to the including of the CD3 signaling domain, the inclusion of co-stimulatory molecules has been shown to enhance CAR T-cell activity in murine models and clinical trials. Several have been investigated including CD28, 4-IBB, ICOS, CD27, CD270, CD30 and OX-40.

In certain embodiments, methods of producing CAR expressing cells are disclosed comprising, or alternatively consisting essentially of: (i) transducing a population of isolated cells with a nucleic acid sequence encoding a CAR and (ii) selecting a subpopulation of cells that have been successfully transduced with said nucleic acid sequence of step (i). In some embodiments, the isolated cells are T-cells, an animal T-cell, a mammalian T-cell, a feline T-cell, a canine T-cell or a human T-cell, thereby producing CAR T-cells. In certain embodiments, the isolated cell is an NK-cell, e.g., an animal NK-cell, a mammalian NK-cell, a feline NK-cell, a canine NK-cell or a human NK-cell, thereby producing CAR NK-cells.

### Therapeutic Applications of CAR T-Cells, CAR N Cells and CAR Macrophages.

The CAR cells, which include the CAR T-cells, the CAR NK cells and the CAR Macrophages described herein, may be used to treat non-gynelocogic AMHRII-expressing tumors. The CAR cells of the present invention are preferably used for treating AMHRII-expressing tumors in patients affected with one cancer described herein. In preferred embodiments, the CAR cells of the present invention are preferably used for treating cancers selected in a group comprising colon cancer, liver cancer, hepatocellular carcinoma, testis cancer, thyroid cancer, gastric cancer, gastrointestinal cancer, bladder cancer, pancreatic cancer, head and neck cancer, kidney cancer, liposarcoma, fibrosarcoma, pleuramesothelioma, melanoma, sarcoma, brain cancer, osteocarcinoma, breast cancer, prostate cancer and leukemia.

The CAR cells of the present invention may be administered either alone or in combination with diluents, known anti-cancer therapeutics, and/or with other components such as cytokines or other cell populations that are immunostimulatory.

Method aspects of the present disclosure relate to methods for inhibiting the growth of a tumor in a subject in need thereof and/or for treating a cancer patient in need thereof. In some embodiments, the tumor is a solid tumor. In some embodiments, the tumors/cancer is thyroid, breast, ovarian or prostate tumors/cancer.

The CAR cells as disclosed herein may be administered either alone or in combination with diluents, known anti-cancer therapeutics, and/or with other components such as cytokines or other cell populations that are immunostimulatory. They may be first line, second line, third line, fourth line, or further therapy. The can be combined with other therapies. Non-limiting examples of such include chemotherapies or biologics. Appropriate treatment regimen will be determined by the treating physician or veterinarian.

Pharmaceutical compositions comprising the CAR of the present invention may be administered in a manner appropriate to the disease to be treated or prevented. The quantity and frequency of administration will be determined by such factors as the condition of the patient, and the type and severity of the patient's disease, although appropriate dosages may be determined by clinical trials.

### Therapeutic Applications

As it is already discloses elsewhere in the present specification, AMHRII-binding agents disclosed herein, which encompass (i) the anti-AMHRII antibodies disclosed herein, (ii) the Antibody Drug Conjugates disclosed herein and (iii) the CAR cells (including the CAR T-cells, the CAR NK cells and the CAR Macrophages) disclosed herein, consist of active ingredients that may be used for preventing or treating non-gynecologic AMHRII-expressing cancers, and especially cancers selected in a group comprising colon cancer, liver cancer, hepatocellular carcinoma, testis cancer, thyroid cancer, gastric cancer, gastrointestinal cancer, bladder cancer, pancreatic cancer, head and neck cancer, kidney cancer, liposarcoma, fibrosarcoma, pleuramesothelioma, melanoma, sarcoma, brain cancer, osteocarcinoma, breast cancer, prostate cancer and leukemia.

Cancer treatment methods that make use of anti-tumor antigen antibodies or anti-tumor antigen CAR cells are well-known from the one skilled in the art.

In some embodiments, cancer patients are tested for determining whether their tumor cells express AMHRII at their surface, before performing a treatment with an AMHRII-binding agent, such as an anti-AMHRII antibody, an anti-AMHRII ADC, an anti-AMHRII CAR T-cell, an anti-AMHRII CAR NL cell or an anti-AMHRII CAR Macrophage.

Such a preliminary test for detecting membrane expression of AMHRII is preferred for the treatment of cancers expressing AMHRII with a low frequency. In contrast, such a preliminary test for detecting membrane expression of AMHRII may not be performed for the treatment of cancers expressing AMHRII at a high frequency.

Thus, in some embodiments, this invention relates to an AMHRII-binding agent as specified herein for its use for preventing or treating an individual affected with an AMHRII-positive cancer selected in a group comprising colon cancer, liver cancer, hepatocellular carcinoma, testis cancer, thyroid cancer, gastric cancer, gastrointestinal cancer, bladder cancer, pancreatic cancer, head and neck cancer, kidney cancer, liposarcoma, fibrosarcoma, pleuramesothelioma, melanoma, sarcoma, brain cancer, osteocarcinoma, breast cancer, prostate cancer, and leukemia.

This invention concerns the use of an AMHRII-binding agent for the preparation of a medicament for preventing or treating an individual affected with an AMHRII-positive cancer selected in a group comprising colon cancer, liver cancer, hepatocellular carcinoma, testis cancer, thyroid cancer, gastric cancer, gastrointestinal cancer, bladder cancer, pancreatic cancer, head and neck cancer, kidney cancer, liposarcoma, fibrosarcoma, pleuramesothelioma, melanoma, sarcoma, brain cancer, osteocarcinoma, breast cancer, prostate cancer and leukemia.

This invention also pertains to a method for preventing or treating an individual affected with an AMHRII-positive cancer selected in a group comprising colon cancer, liver cancer, hepatocellular carcinoma, testis cancer, thyroid cancer, gastric cancer, gastrointestinal cancer, bladder cancer, pancreatic cancer, head and neck cancer, kidney cancer, liposarcoma, fibrosarcoma, pleuramesothelioma, melanoma, sarcoma, brain cancer, osteocarcinoma, breast cancer, prostate cancer and leukemia, wherein the said method comprises a step of administering to the said individual an anti-AMHRII binding agent.

An individual may be assigned as being an individual affected with an AMHRII-positive cancer by performing a method of detecting cell surface AMHRII protein expression on a cancer tissue sample previously obtained from the said individual. Detection of cell surface AMHRII protein expression may be performed according to a variety of methods that are well known from the one skilled in the art. Cell surface AMHRII protein expression detection methods notably encompass immunohistochemistry methods as well as fluorescence activated cell sorting methods that are illustrated in the examples herein.

This invention also relates to a method for determining whether an individual is eligible (i.e. responsive) to a cancer treatment with an AMHRII-binding agent, wherein the said method comprises the step of determining whether a tumor tissue sample previously obtained from the said individual express the AMHRII protein at the cell surface.

Thus, this invention also relates to a method for determining whether an individual which is affected with a cancer selected in a group comprising colon cancer, liver cancer, hepatocellular carcinoma, testis cancer, thyroid cancer, gastric cancer, gastrointestinal cancer, bladder cancer, pancreatic cancer, head and neck cancer, kidney cancer, liposarcoma, fibrosarcoma, pleuramesothelioma, melanoma, sarcoma, brain cancer, osteocarcinoma, breast cancer, prostate cancer and leukemia, is eligible to a cancer treatment with an AMHRII-binding agent, i.e. is responsive to a cancer treatment with an AMHRII-binding agent, wherein the said method comprises the steps of:
a) determining if cancer cells from the said patient express AMHRII at their membrane, and
b) concluding that the said patient is eligible to a cancer treatment with an AMHRII-binding agent, i.e. is responsive to a cancer treatment with an AMHRII-binding agent if membrane expression of AMHRII by the said cancer cells has been determined at step a).

In preferred embodiments of the said method, it is concluded at step b) that the said patient is eligible (i.e. responsive) to a cancer treatment with an AMHRII-binding agent when (i) a AMHRII expression score value is determined at step a) and when (ii) the said AMHRII expression score value is of a threshold score value or more. The AMHRII score value is most preferably calculated by using the formula (I) described elsewhere in the present specification.

Thus, according to preferred embodiments, step a) of the method is performed by a immunohistochemical method, such as shown in the examples herein.

The cancer cells that are used at step a) generally originate from a biopsy tissue sample that has previously been collected from the said cancer patient.

Preferably, step a) is performed by using an anti-AMHRII antibody selected among those specifically described in the present specification, and notably a 3C23K antibody, the AMHRII binding of which may be detected by using a secondary labeled antibody according to well-known antibody detection techniques, such as those disclosed in the examples herein.

Preferably, a patient affected with a cancer comprised in the above-listed group of cancers is determined as being eligible to a cancer treatment with an AMHRII-binding agent, i.e. is determined as being responsive to a cancer treatment with an AMHRII-binding agent, when a AMHRII expression score value of 1.0 or more, and most preferably a AMHRII expression score value of 1.5 or more is determined in a cancer cell sample originating from the said cancer patient, when performing a scoring method allowing determination of the E-SCORE value according to the formula (I) below:

E-SCORE=FREQ×AMHRII_LEVEL, wherein
E-SCORE means the AMHRII expression score value for a given cancer cell sample,
FREQ means the frequency of the cells contained in the said cancer cell sample for which membrane AMHRII expression is detected, and
AMHRII_LEVEL means the level of expression of AMHRII by the AMHRII-expressing cells contained in the said given cancer cell sample.

The present invention further relates to a method for treating a patient affected with a cancer selected in a group comprising colon cancer, liver cancer, hepatocellular carcinoma, testis cancer, thyroid cancer, gastric cancer, gastrointestinal cancer, bladder cancer, pancreatic cancer, head and neck cancer, kidney cancer, liposarcoma, fibrosarcoma, pleuramesothelioma, melanoma, sarcoma, brain cancer, osteocarcinoma, breast cancer, prostate cancer and leukemia. wherein the said method comprises the steps of:
a) determining whether a tumor tissue sample previously obtained from the said individual express the AMHRII protein at the cell surface, and
b) treating the said individual with an AMHRII-binding agent if the cell surface expression of AMHRII has been determined at step a).

In some preferred embodiments, AMHRII expression is determined at step a) when the said tumor sample has an AMHRII expression score value “E-SCORE” calculated according to the above-described formula (I) of 1.0 or more, which encompasses an E-SCORE value of 1.5 or more.

In most preferred embodiments of the method above, the said AMHRII-binding agent consists of an anti-AMHRII antibody or fragment thereof as specified herein, or of a CAR cell (e.g. a CAR T-cell or a CAR NK-cell) as specified herein.

In some embodiments, the said AMHRII-binding agent is used as the sole anti-cancer active ingredient.

In some other embodiments, the anti-cancer treatment with the said AMHRII-binding agent also comprises subjecting the said individual to one or more further anti-cancer treatments, which include radiotherapy treatment and chemotherapeutic treatment.

Thus, according to such other embodiments, the anti-cancer treatment with the said AMHRII-binding agent also comprises the administration to the said individual of one or more further anti-cancer active ingredients.

Thus, according to some embodiments of a AMHRII-binding agent for its use as described herein, the said AMHRII-binding agent is combined with another anti-cancer treatment, such as combined with one or more other anti-cancer active agent(s).

In some of these embodiments, such a combined therapy may be performed by administering to the cancer patient a pharmaceutical composition comprising a combination of (i) a AMHRII-binding agent, e.g. an anti-AMHRII antibody, and (ii) one or more other anticancer agents.

However, in most embodiments of such a combined therapy, the said cancer patient is administered a combined therapy comprising (i) a AMHRII-binding agent, e.g. an anti-AMHRII antibody, and (ii) one or more other anticancer agents, wherein the said AMHRII-binding agent and the said one or more other anticancer agents are comprised in distinct compositions and are administered separately.

In some embodiments of such a combined treatment, these distinct compositions are administered separately but simultaneously or almost simultaneously.

In some other embodiments of such a combined treatment, these distinct compositions are not administered simultaneously or almost simultaneously but rather separately over a period of time, such as sequentially.

An “anticancer agent” is defined as any molecule that can either interfere with the biosynthesis of macromolecules (DNA, RNA, proteins, etc.) or inhibit cellular proliferation, or lead to cell death by apoptosis or cytotoxicity for example. Among the anticancer agents, there may be mentioned alkylating agents, topoisomerase inhibitors and intercalating agents, anti-metabolites, cleaving agents, agents interfering with tubulin, monoclonal antibodies.

According to a particular aspect, the invention relates to a pharmaceutical composition comprising, as active ingredient, in combination with a pharmaceutically acceptable vehicle, an anticancer agent and an antibody binding to AMHR-II, and especially an anti-AMHRII antibody described herein.

A “pharmaceutically acceptable vehicle” refers to a non-toxic material that is compatible with a biological system such as a cell, a cell culture, a tissue or an organism.

In some embodiments, the invention relates to a pharmaceutical composition comprising, as active ingredient, in combination with a pharmaceutically acceptable vehicle, an anticancer agent, and an antibody binding AMHR-II, and especially an anti-AMHRII antibody described herein.

In some embodiments, the invention relates to a pharmaceutical composition comprising, as active ingredient, in combination with a pharmaceutically acceptable vehicle, an anticancer agent, and an antibody binding AMHR-II, in which the anticancer agent is selected in a group comprising docetaxel, cisplatin, gemcitabine and a combination of cisplatin and gemcitabine.

Other anti-cancer agents that may be used with an anti-AMHRII antibody encompass those which are expected to be active against the given non-gynecologic cancer affecting the patient to be treated. Illustratively, as shown in the examples herein, a patient affected with a colorectal cancer may be treated with an anti-AMHRII antibody which is combined with another anticancer agent which is already known to be active against colorectal cancer. Further illustratively, a patient affected with a metastatic colorectal cancer may be treated with an anti-AMHRII antibody which is combined with another anticancer agent which is already known to be active against colorectal cancer, or alternatively against metastatic colorectal cancer.

Other anti-cancer agents include nucleoside analogues and nucleotide analogues. Nucleoside analogues encompass deoxyadenosine analogues, adenosine analogues, deoxycycline analogues, guasonine analogues, deoxyguanosine analogues, thymidine analogues, deowythymidine analogues and deoxyuridine analogues. Among deoxycytidine analogues, it may be cited cytarabine and gemcitabine. Among deoxyuridine analogues, it may be cited idoxuridine and trifluridine.

Illustratively, as it is shown in the examples herein, patients affected with a colorectal cancer, including patients affected with a metastatic colorectal cancer, may be treated with a therapy combining an anti-AMHRII antibody and a deoxyuridine analogue such as trifluridine.

In some embodiments, a trifluridine-containing anticancer agent may comprise a combination of trifluridine and an agent preventing or reducing trifluridine degradation. An agent preventing or reducing trifluridine degradation encompasses a thymidine phosphorylase inhibitor such as tipiracil. Illustratively, a trifluridine-containing anticancer agent may consist of a composition comprising trifluridine and tipiracil, such as that available under the name of Lonsurf®.

To date, Lonsurf® is available according to two distinct formulations, (i) a formulation comprising 15 mg trifluridine and 6.14 mg tipiracil and (ii) a formulation comprising 20 mg trifluridine and 18.9 mg tipiracil.

The one skilled in the art may indifferently use either one or the other of these two formulations, according, notably, to the weight and/or body size (and thus body surface area) of the subject to be treated, and consequently according to the amount of the active ingredient to be administered.

Other anti-cancer agents that may be used in combination with an anti-AMHRII antibody encompass paclitaxel or a platinum salt such as oxaliplatin, cisplatin and carboplatin.

The anticancer agent may also be selected from chemotherapeutic agents other than the platinum salts, small molecules, monoclonal antibodies or else anti-angiogenesis peptibodies.

The chemotherapeutic agents other than the platinum salts include the intercalating agents (blocking of DNA replication and transcription), such as the anthracyclines (doxorubicin, pegylated liposomal doxorubicin), the topoisomerase inhibitors (camptothecin and derivatives: Karenitecin, topotecan, irinotecan), or else SJG-136, the inhibitors of histone deacetylase (vorinostat, belinostat, valproic acid), the alkylating agents (bendamustine, glufosfamide, temozolomide), the anti-mitotic plant alkaloids, such as the taxanes (docetaxel, paclitaxel), the vinca alkaloids (vinorelbine), the epothilones (ZK-Epothilone, ixabepilone), the anti-metabolites (gemcitabine, elacytarabine, capecitabine), the kinesin spindle protein (KSP) inhibitors (ispinesib), trabectedin or else ombrabulin (combretastatin A-4 derivative).

Among the small molecules there are the poly(ADP-ribose)polymerase (PARP) inhibitors: olaparib, iniparib, veliparib, rucaparib, CEP-9722, MK-4827, BMN-673, the kinase inhibitors, such as the tyrosine kinase inhibitors (TKI) among which there may be mentioned the anti-VEGFR molecules (sorafenib, sunitinib, cediranib, vandetanib, pazopanib, BIBF 1120, semaxanib, Cabozantinib, motesanib), the anti-HER2/EGFR molecules (erlotinib, gefitinib, lapatinib), the anti-PDGFR molecules (imatinib, BIBF 1120), the anti-FGFR molecules (BIBF 1120), the aurora kinase/tyrosine kinase inhibitors (ENMD-2076), the Src/Abl kinase inhibitor (Saracatinib), or also Perifosine, Temsirolimus (mTOR inhibitor), alvocidib (cyclin-dependent kinase inhibitor), Volasertib (inhibitor of PLK1 (polo-like kinase 1) protein, LY2606368 (inhibitor of checkpoint kinase 1 (chk 1), GDC-0449 (Hedgehog Pathway Inhibitor), Zibotentan (antagonist of the ETA-receptor), Bortezomib, Carfilzomib (proteasome inhibitor), cytokines such as IL-12, IL-18, IL-21, INF-alpha, INF-gamma.

Among the antibodies, there may be mentioned, the anti-VEGF: bevacizumab, the anti-VEGFR: ramucirumab, the anti-HER2/EGFRs: trastuzumab, pertuzumab, cetuximab, panitumumab, MGAH22, matuzumab, anti-PDGFR alpha: IMC-3G3, the anti-folate receptor: farletuzumab, the anti-CD27: CDX-1127, the anti-CD56: BB-10901, the anti-CD105: TRC105, the anti-CD276: MGA271, the anti-AGS-8: AGS-8M4, the anti-DRS: TRA-8, the anti-HB-EGF: KHK2866, the anti-mesothelins: amatuximab, BAY 94-9343 (immunotoxin), catumaxomab (EpCAM/CD3 bispecific antibody), the anti-IL2R: daclizumab, the anti-IGF-1R: ganitumab, the anti-CTLA-4: ipilimumab, the anti-PD 1: nivolumab and pembrolizumab, the anti-CD47: Weissman B6H12 and Hu5F9, Novimmune 5A3M3, INHIBRX 2A1, Frazier VxP037-01LC1 antibodies, the anti-Lewis Y: Hu3S193, SGN-15 (immunotoxin), the anti-CA125: oregovomab, the anti-HGF: rilotumumab, the anti-IL6: siltuximab, the anti-TR2: tigatuzumab, the anti-alpha5 beta1 integrin: volociximab, the anti-HB-EGF: KHK2866. The anti-angiogenesis peptibodies are selected from AMG 386 and CVX-241.

More particularly, it is described herein a pharmaceutical composition comprising, as active ingredient, in combination with a pharmaceutically acceptable vehicle, an anticancer agent, and an antibody binding AMHR-II, in which the anticancer agent is selected in a group comprising docetaxel, cisplatine, gemcitabine and a combination of cisplatine and gemcitabine.

Even more particularly, it is described herein a pharmaceutical composition comprising, as active ingredient, in combination with a pharmaceutically acceptable vehicle, an anticancer agent, and an antibody binding AMHR-II, in which the mutated humanized monoclonal antibody termed 3C23K herein and the anticancer agent is selected in a group comprising docetaxel, cisplatine, gemcitabine and a combination of cisplatine and gemcitabine.

An AMHRII-binding agent as disclosed herein, and especially an anti-AMHRII antibody disclosed herein, may administered in various ways, which include oral administration, subcutaneous administration, and intravenous administration.

The term “therapeutically effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, duration of progression free survival (PFS), the response rates (RR), duration of response, and/or quality of life.

Therapeutic formulations of the agents (e.g., antibodies) used in accordance with the invention are prepared for storage by mixing an antibody having the desired degree of purity with optional pharmaceutically acceptable carriers, excipients or stabilizers {Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes {e.g. Zn-protein complexes); and/or non-ionic surfactants such as TWEEN™, PLURONICS™ or polyethylene glycol (PEG).

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration may be sterile. This is readily accomplished by filtration through sterile filtration membranes.

In another particular aspect, the invention relates to a composition for use as a medicinal product in the prevention or treatment of a non-gynecologic cancer described herein, comprising an anticancer agent and an antibody binding AMHR-II, in a formulation intended for administration by the intravenous or intraperitoneal route.

In another particular aspect, the invention relates to a composition for use as a medicinal product in the prevention or treatment of a non-gynecologic cancer described herein, comprising an anticancer agent and an antibody binding AMHR-II, the monoclonal antibody and the anticancer agent being intended for separate, simultaneous or sequential administration.

The antibody and the anticancer agent may be combined within one and the same pharmaceutical composition, or may be used in the form of separate pharmaceutical compositions, which may be administered simultaneously or sequentially. In particular, the products may be administered separately, namely either concomitantly, or independently, for example with a time gap.

Illustrative embodiments are shown in the examples herein wherein the AMHRII-binding agent and another anticancer agent are contained in separate composition and are administered simultaneously or almost simultaneously to the cancer patient, according to a given administration schedule.

As shown in the examples herein, a combined therapy comprising administering (i) AMHRII-binding agent, e.g. an anti-AMHRII antibody, and (ii) another anticancer agent may be administered according to a monthly schedule, wherein each of the agents are administered separately, each during a specific period of time of a monthly administration cycle.

In some embodiments, the present invention pertains to a method of treating a non-gynecologic cancer in an individual in need thereof, comprising the steps of:
a) administering a AMHRII-binding agent, such as an anti-AMHRII antibody, and
b) administering another anticancer agent,
   wherein administration steps a) and b) are performed simultaneously or sequentially.

This invention notably relates to a method of treating a colorectal cancer in an individual in need thereof, comprising the steps of:
a) administering an anti-AMHRII antibody,
b) administering trifluridin, optionally combined with a thymidine phosphorylase inhibitor such as tipiracil,
   wherein administration steps a) and b) are performed simultaneously or sequentially.

Most preferably, steps a) and b) of the method are performed sequentially and at distinct period of time intervals.

This invention further relates to a method of treating a colorectal cancer in an individual in need thereof, comprising the steps of:
a) administering an anti-AMHRII antibody,
b) administering trifluridin combined with a thymidine phosphorylase inhibitor such as tipiracil,
   wherein administration steps a) and b) are performed sequentially.

This invention still further relates to a method of treating a colorectal cancer in an individual in need thereof, comprising performing one or more monthly cycles of treatment, wherein each monthly cycle of treatment comprises the steps of:
a) weekly administering an anti-AMHRII antibody,
b) twice daily administering trifluridin combined with a thymidine phosphorylase inhibitor such as tipiracil,
   wherein administration steps a) and b) are reiterated during the selected number of monthly cycles of treatment.

A monthly cycle of treatment is most preferably a 28 days cycle of treatment.

This invention yet further relates to a method of treating a colorectal cancer in an individual in need thereof, comprising performing one or more monthly cycles of treatment, wherein each monthly cycle of treatment comprises the steps of:
a) weekly administering an anti-AMHRII antibody from Day 1 to Day 15 of the said monthly cycle of treatment,
b) twice daily administering trifluridin combined with a thymidine phosphorylase inhibitor such as tipiracil, from Day 1 to Day 5 and then from Day 8 to Day 12 of the said monthly cycle of treatment,
   wherein administration steps a) and b) are reiterated during the selected number of monthly cycles of treatment.

A monthly cycle of treatment is most preferably a 28 days cycle of treatment.

In preferred embodiments, the above method further comprises step c) of reiterating steps a) and b) during a time period which is appropriate for achieving completion of the cancer treatment of the said individual. Step c) may comprise one or more cycles of reiteration of steps a) and b), such as one or more monthly cycles of reiteration of steps a) and b).

In some embodiments of the above method, step a) is performed at least once weekly, such as at least twice weekly.

In some embodiments of the above method, step b) is performed at least once weekly, preferably at least twice weekly and most preferably at least once daily. In some embodiments of the method, step b) is performed twice daily.

In some embodiments of the above method, steps a) and b) are reiterated according to one or more monthly cycles of treatment.

In some embodiments of the above method, step a) of administering an anti-AMHRII antibody is performed weekly during the whole period of time of the treatment.

In some preferred embodiments; the said anti-AMHRII antibody is selected from the group of anti-AMHRII antibodies described herein, such as the anti-AMHRII antibody termed “3C23K” herein. In some preferred embodiments, the said anti-AMHRII antibody is administered weekly at a dose ranging from 5 mg/kg body weight to 20 mg/kg body weight, such as at a dose of 7 mg/kg body weight or 10 mg/kg body weight.

In some embodiments of the above method, step b) of administering trifluridin, optionally combined with a thymidine phosphorylase inhibitor such as tipiracil, is performed twice daily, from Day 1 to Day 5 and then from Day 8 to Day 12 of a monthly cycle of treatment, most preferably of a 28 days cycle of treatment.

Step c) of the method, when present, comprises one or more monthly cycles, or alternatively 28 days cycles, of reiteration of steps a) and b), such as two or more, three or more, four or more, five or more, six or more, seven or more, eight or more, nine or more, or ten or more monthly cycles of reiteration of steps a) and b).

More particularly, the invention relates to a composition for use as a medicinal product in the prevention or treatment of a non-gynecologic cancer described herein, comprising an anticancer agent and an antibody binding AMHR-II, in which the antibody and the anticancer agent are combined within the same pharmaceutical composition.

According to another particular aspect, the invention relates to a composition for use as a medicinal product in the prevention or treatment of a non-gynecologic cancer described herein, comprising an anticancer agent and an antibody binding AMHR-II, in which the therapeutically effective quantity of the anti-AMHRII antibody administered to a patient is in a range of from 1 mg/kg body weight to 100 mg/kg body weight, advantageously from 5 mg/kg body weight to 20 mg/kg body weight, such as for example at 7 mg/kg body weight or 10 mg/kg body weight.

According to another particular aspect, the invention relates to a composition for use as a medicinal product in the prevention or treatment of a non-gynecologic cancer described herein, comprising an anticancer agent and an antibody binding AMHR-II, in which the therapeutically effective quantity of anticancer agent administered to a patient is in a range from about 10 mg to about 700 mg, preferably in a range from about 20 mg to about 350 mg, and preferably about 110 mg.

According to another particular aspect, the invention relates to a composition for use as a medicinal product in the prevention or treatment of a non-gynecologic cancer described herein, comprising an anticancer agent and an antibody binding AMHR-II, in which the therapeutically effective quantity of antibody administered to a patient is about 70 mg and the dose of anticancer agent administered to the patient is about 110 mg.

The present invention is further illustrated by, without in any way being limited to, the examples below.

### EXAMPLES

### Example 1: Differential AMHRII Gene Expression and AMHRII Protein Expression

### A. Materials and Methods

### A.1. Cell Lines and Cultures

The COV434 WT cell line (ECACC No 07071909) was maintained in DMEM/GlutaMax (Gibco) supplemented with 10% FBS, penicillin 100 U/ml and Streptomycin 100 μg/ml. Geneticin (Gibco) at 400 μg/ml was added for the COV434 MISRII transfected cell line. The erythroleukemia K562 cell line (ATCC® CCL-243™) was cultivated in suspension in IMDM medium (Sigma-Aldrich) supplemented with 10% FBS and penicillin/Streptomycin and maintained at a density between 1×10⁵ and 1×10⁶ cells/ml in T75 flasks. The OV90 cell line (ATCC® CRL-11732™, ovary serous adenocarcinoma) was cultivated in a mixture 1:1 of MCDB 105 medium (Sigma-Aldrich) containing a final concentration of 1.5 g/l sodium bicarbonate and medium 199 (Sigma-Aldrich) containing a final concentration of 2.2 g/l sodium bicarbonate supplemented with 15% FBS and penicillin/Streptomycin. The NCI-H295R cell line (adrenocortical carcinoma, ATCC® CRL-2128™) was maintained in DMEM:F12 medium (Sigma-Aldrich) supplemented with iTS⁺Premix (Corning), 2.5% Nu-Serum (Falcon) and penicillin/Streptomycin. Cells were grown at 37° C. in a humidified atmosphere with 8% CO2 and medium was replaced one or twice a week depending the cell lines.

A.2. Relative Quantification of AMHR2 mRNA by RT-qPCR

Extraction of RNA.

Total RNA from 1-5×10⁶ cells pellet was prepared using Trizol® Plus RNA Purification Kit (Ambion) according to the manufacturer's instructions. Briefly, after phenol/chloroform extraction, RNA of lysed cells was adsorbed on silica matrix, DNAse treated, then washed and eluted with 30 μl of RNAse free water. RNA concentrations and quality were assessed with spectrophotometer (NanoDrop, ThermoFisher Scientific).

cDNA Synthesis.

RNA (1 μg) was reverse transcribed using Maxima H Minus First Strand cDNA Synthesis Kit (Ambion) and oligo-dT primers by incubation 10 min at 25° C. for priming and 15 min at 50° C. for reverse transcription followed by 5 min at 85° C. for reverse transcriptase inactivation.

Quantitative PCR.

Quantitative PCR was performed in Light Cycler 480 (Roche) in 96-wells microplates using Luminaris Color HiGreen qPCR Master Mix (Ambion) in a final volume of 20 μl. The following primers were used: for AMHR2, Forward 5′-TCTGGATGGCACTGGTGCTG-3′ (SEQ ID NO. 71) and Reverse 5′-AGCAGGGCCAAGATGATGCT-3′ (SEQ ID NO. 72), for TBP, Forward 5′-TGCACAGGAGCCAAGAGTGAA-3′ (SEQ ID NO. 73) and Reverse 5% CACATCACAGCTCCCCACCA-3′ (SEQ ID NO. 74). Amplications were performed using cDNA template (100 ng equivalent RNA) and the following protocol: UDG pretreatment 2 min at 50° C., denaturation 10 min at 95° C. followed by 40 cycles of 15 s at 95° C./30 s at 60° C./30 s at 70° C. A melting curves analysis was performed at the end of each experiments to control the absence of genomic DNA and dimer primer. Each cDNA samples and controls (“no template sample” and “no reverse transcript RNA”) were tested in duplicate. The mean values of Cycle Threshold (Ct) were calculated and the AMHR2 relative quantification (RQ) was expressed as 2^{-ΔΔCt} where ΔΔCt=ΔCtₛₐₘₚₗₑ−ΔCt_{calibrator} and ΔCt=Ct_{AMHR2}−Ct_{TBP}. HCT116 sample was used as calibrator and TBP as housekeeping gene for normalization.

Table 2 below depicts the AMHRII expression level in the tested cell lines using the Q-PCR method described above.

TABLE 2

| | Mean Ct | Mean Ct | |
|---|---|---|---|
| Cell line | amhr2 | TBP | RQ |
| HCT116 | 34.27 | 22.25 | 1 |
| COV434 WT | 31.34 | 22.82 | 11.3 |
| K562 | 25.31 | 21.36 | 268.7 |
| NCI-H295R | 26.16 | 22.83 | 413.0 |
| OV90 | 25.65 | 22.67 | 526.4 |

### A.3. Evaluation of Membrane AMHR2 Expression by Flow Cytometry Analysis.

For Fluorescent-Activated Cell Sorting (FACS) analysis, 4×10⁵ cells were incubated with 25 μg/ml of 3C23K for 30 min at 4° C. After washes with PBS-BSA2%, the primary antibody was detected by an anti-species secondary antibody conjugated to a fluorophore. The 3C23K was detected by an anti-human F(ab′)₂ conjugated to Phycoerythrin (1:1000, Beckman-Coulter, IM0550). After washes with PBS, FACS analysis of the resuspended cells was realized in the FL2 channel of the BD Accuri™ C6 flow cytometer (BD Bioscience).

### B. Results

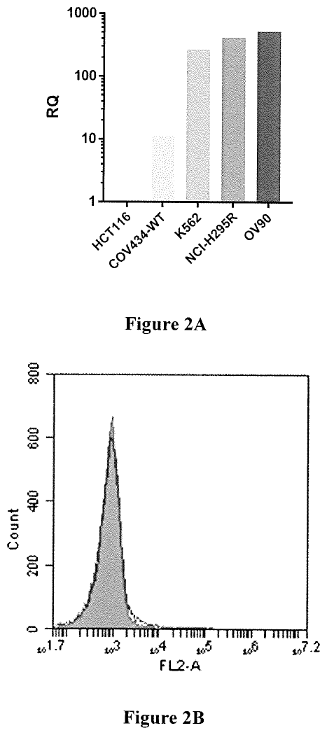

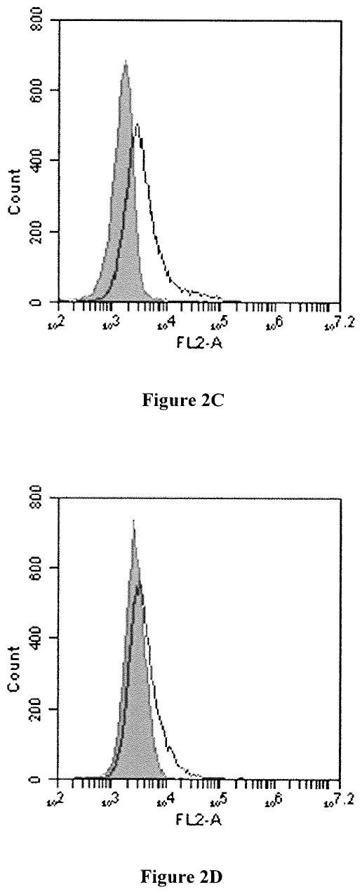

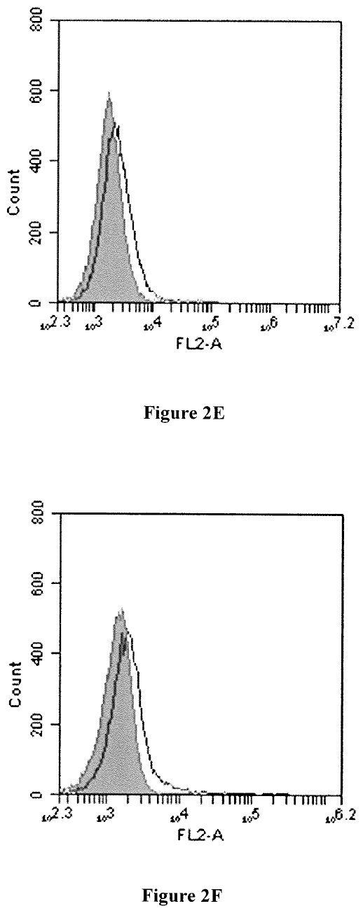

The results are depicted in FIG. 2. The results showed that the recombinant cell line COV434-WT (about 3% of the AMHRII gene expression level measured for the cell line NCI-H295R) although the COV434-WT cell line has a significative membrane expression level of human AMHRII protein.

These results showed that there is strictly no correlation between AMHRII gene expression and membrane AMHRII protein expression.

### Example 2: AMHRII Expression in Non-Gynecologic Cancers (Human Tumor Samples)

### A. Materials and Methods

### A.1. Objective

Immunohistochemical study of human cancer cells xenografts in mice (PDXs) for detecting anti-Müllerian hormone receptor type 2 (AMHR2) expression using a biotinylated 3C23K monoclonal antibody.

### A.2. Protocol and Methodology

The cell lines: fixed in formaldehyde acetic acid alcohol (AFA) with constitution of cellblocks
Human Tumors: fixation in formalin for external samples and in AFA for slides from Curie Institute
Immunohistochemistry (IHC) technique was possible after dewaxing samples and unmasking at pH9 (microwave EZ Retriever 15′ at 90° C., followed by cooling during 20′).
Anti-Mullerian Hormone Receptor Type II detection by immunoperoxidase technique and DAB chromogenic substrate revelation.
After blocking endogenous peroxydase activity, the slides were incubated with diluted biotinylated primary antibody ( 1/800, 8 μg/mL) for 90 minutes at room temperature. The tissue sections were then washed with PBS and incubated with avidin/biotin ABC [Vector] complex for 30 minutes. Immunoreactive signals were detected using DAB substrate solution (DAB+ Substrate buffer/Liquid DAB+ chromogen, 10 minutes incubation). Finally, the sections were lightly counterstained with Mayer's Hematoxylin (Lillie's Modification).
Negative controls were obtained by substitution of the primary antibodies with isotype control immunoglobulin (R565) or with antibody diluent alone (negative buffer control) in the immunohistochemical staining procedure.
Positive controls were obtained by using AMHR2-transfected COV434 cells and human granulosa tumor samples
After processing, sections were observed by digitalization via Philips IMS. All specimens were scored independently by 2 pathologists.
Localization of the labeling was detailed: cytoplasmic and/or membranous.
Intensity was classified as unequivocal brown labeling of tumor cell membrane and/or cytoplasm through the following scoring system: intensity of the labeling was defined as 0 for negative, 1 for weak, 2 for moderate, and 3 for strong as shown in the COV434 positive control.
Frequency was defined as a percentage of cells expressing AMHRII. Necrotic areas were excluded from analysis. The Global Histological score was established by using frequency×mean of intensity scores (0 to 3) cumulating membranous and cytoplasmic expression.
All slides were duly stored.

### B. Results

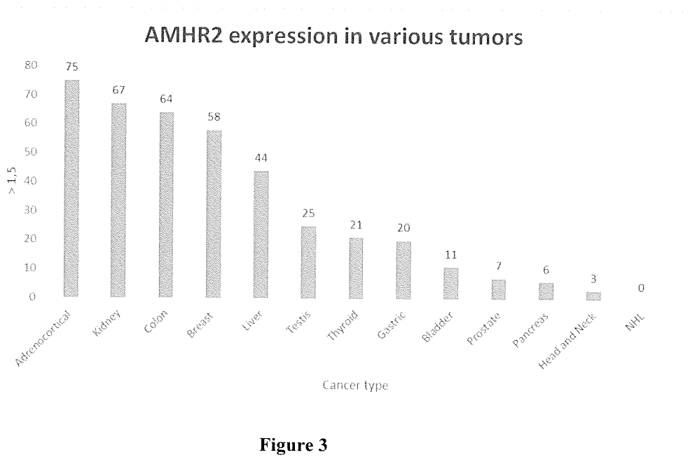

The results of AMHRII membrane expression by various primary human cancer cells are also depicted in FIG. 3, wherein the AMHRII expression score is represented for a panel of distinct cancer cell types.

The results are depicted in FIG. 3. The results showed that AMHRII is expressed at the cell surface in a plurality of various non-gynecologic human cancers including colon cancer, liver cancer, testis cancer, thyroid cancer, gastric cancer, bladder cancer, pancreatic cancer, as well in head and neck cancer.

### Example 3: AMHRII Expression in Non-Gynecologic Cancers (Human Tumor Xenografts)

### A. Materials and Methods

### A.1. Objective

Immunohistochemical study of human cancer cells xenografts in mice (PDXs) for detecting anti-Müllerian hormone receptor type 2 (AMHR2) expression using a biotinylated 3C23K monoclonal antibody.

### A.2. Protocol and Methodology

The cell lines: fixed in formaldehyde acetic acid alcohol (AFA) with constitution of cellblocks
Human Tumors: fixation in formalin for external samples and in AFA for slides from Curie Institute
Immunohistochemistry (IHC) technique was possible after dewaxing samples and unmasking at pH9 (microwave EZ Retriever 15′ at 90° C., followed by cooling during 20′).
Anti-Mullerian Hormone Receptor Type II detection by immunoperoxidase technique and DAB chromogenic substrate revelation.
After blocking endogenous peroxydase activity, the slides were incubated with diluted biotinylated primary antibody ( 1/800, 8 μg/mL) for 90 minutes at room temperature. The tissue sections were then washed with PBS and incubated with avidin/biotin ABC [Vector] complex for 30 minutes. Immunoreactive signals were detected using DAB substrate solution (DAB+ Substrate buffer/Liquid DAB+ chromogen, 10 minutes incubation). Finally, the sections were lightly counterstained with Mayer's Hematoxylin (Lillie's Modification).
Negative controls were obtained by substitution of the primary antibodies with isotype control immunoglobulin (R565) or with antibody diluent alone (negative buffer control) in the immunohistochemical staining procedure.
Positive controls were obtained by using AMHR2-transfected COV434 cells and human granulosa tumor samples
After processing, sections were observed by digitalization via Philips IMS. All specimens were scored independently by 2 pathologists.
Localization of the labeling was detailed: cytoplasmic and/or membranous.
Intensity was classified as unequivocal brown labeling of tumor cell membrane and/or cytoplasm through the following scoring system: intensity of the labeling was defined as 0 for negative, 1 for weak, 2 for moderate, and 3 for strong as shown in the COV434 positive control.
Frequency was defined as a percentage of cells expressing AMHRII. Necrotic areas were excluded from analysis. The Global Histological score was established by using frequency×mean of intensity scores (0 to 3) cumulating membranous and cytoplasmic expression.
All slides were duly stored.

### B. Results

### a) Controls

The negative control and isotype control were devoid of reactivity on tumor cells.
The positive control sample (COV434 AMHRII amplified) showed a diffuse immunostaining of cells (intensity score: 3). The labeling was homogeneous (frequency score: 100%) with cytoplasmic and membranous localization.
The positive Granulosa control sample showed a strong immunostaining of tumor cells (intensity score 3). The labeling was homogeneous (frequency score: 100%) with cytoplasmic and membranous localization.

### b) Screening of Patient-Derived Xenografts (PDX) Samples.

It is important to notice that membranous expression of AMHR2 seems to be underestimated when samples are fixed in formalin in comparison to samples processed in AFA.

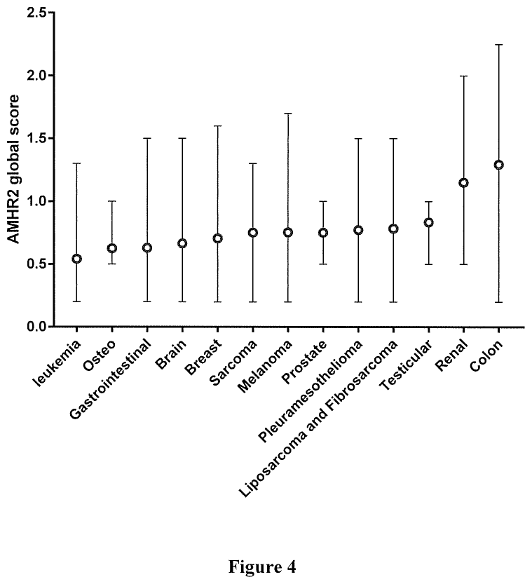

The results of AMHRII membrane expression by various human tumors xenografted in mice are depicted in FIG. 4, wherein the AMHRII expression score is represented for a panel of distinct cancer cell types.

Part of the results of AMHRII expression by human tumor xenografts are summarized in Table 3 hereunder.

TABLE 3
AMHRII expression in human tumor xenografts

| | Positivity in tumors | |
|---|---|---|
| | (percent of positive | number of PDXs |
| Tumor type | PDXs) | tested |
| Colon | 35% | 6 |
| Liver | 44% | 3 |
| Kidney | 84% | 13 |

### c) Conclusions

AMHR2 protein expression was confirmed for 4 out of 6 PDX models positive for AMHR2 transcription. These PDXs were adapted from glioma (ODA14-RAV), and colon (TC306-BAU) cancers. Levels of expression were moderate but significant, characterized by global score of 1 to 1.5. These data suggest that other than gynecological cancer could express AMHR2.

These models could be used for characterizing anti-AMHR2 therapies in the future.

### Example 4: In Vivo Efficacy of Anti-AMHRII Antibodies Against AMHRII-Expressing Non-Gynecologic Cancers

### A. Materials and Methods

### A.1. Abbreviations

Commonly used abbreviation in this protocol is shown in both Table 4 and Table 5.

TABLE 4
Dosing related abbreviations
Dosing schedule

| | |
|---|---|
| Bid | Twice daily |
| Qd | Every day |
| Q2d | Every other day (Qod as well) |
| Q3d | Every tree days (one day dosing and 2 days off) |
| Q4d | Every four days (one day dosing and 3 days off) |
| BIW | Twice weekly |
| QW | Every week |
| Q3W | Every three weeks |

Route of administration (ROA)

| | |
|---|---|
| i.p. | Intraperitoneal (ly) |
| i.v. | Intravenous(ly) |
| p.o. | Oral(ly) |
| s.c. | Subcutaneous(ly) |

TABLE 5
Other common abbreviation used in this example

| Abbreviations | Full-text & descriptions |
|---|---|
| ANOVA | Analysis of variance |
| BW | Body weight |
| BWL | Body weight loss |
| GLP | Good Laboratory Practice |
| MTD | Maximum tolerated dose |
| MN | Mean tumor volume |
| TV | Tumor volume |
| TGI | Tumor growth inhibition, % TGI = (1 − (Ti − T0)/(Vi − V0))*100; Ti as the mean |
| | tumor volume of the treatment group on the measurement day; T0 as the mean tumor |
| | volume of the treatment group at D1; Vi as the mean tumor volume of control |
| | group at the measurement day; V0 as the tumor volume of the control group at |
| | D1. |
| T-C | T-C is calculated with T as the time (in days) required for the mean tumor size of |
| | the treatment group to reach a predetermined size (e.g., 1000 mm³), and C is |
| | the time (in days) for the mean tumor size of the control group to reach the |
| | same size. |
| T/C | The T/C value (%) is an indicator of tumor response to treatment, and one of |
| | commonly used anti-tumor activity endpoint; T and C are the mean tumor |
| | volume of the treated and control groups, respectively, on a given day. |
| REG | REG(%) values are calculated using the formula: % REG = [(VTr_{day 0} − |
| | VTr_{day x})/VTr_{day 0}] × 100%. |
| SOC | Standard of care used in clinic setting for a specific disease |
| FFPE | Formalin fixed paraffin embedded |

### A.2. Study Objective

To evaluate preclinically the in vivo efficacy of GamaMabss' anti-AMHR2 monoclonal antibody, named GM102 in the treatment of Huprime® HCC xenograft model LI1097 in Balb/C nude mice. The model LI1097 was selected after a screening for AMHR2 transcription processed by CrownBio, using RNAseq (transcriptome sequencing). Further, AMHR2 membranous protein expression of this model was confirmed by Institut Curie, France, using IHC.

### A.3. Experimental Design

TABLE 6
Study design of efficacy study

| | | | Dose | | |
|---|---|---|---|---|---|
| | | | level | Dose | Dosing |
| Group | N | Treatment | (mg/kg) | Route | Frequency |
| 1 | 8 | Vehicle (Solvant control) | — | i.v. | BIW × 4 weeks |
| 2 | 8 | GamaMabs's Ab | 20 mg/kg | i.v. | BIW × 4 weeks |
| 3 | 8 | GamaMabs's Ab | 50 mg/kg | i.v. | BIW × 4 weeks |
| 4 | 8 | Sorafenib | 50 mg/kg | p.o. | QD × 4 weeks |

Note:
N: animal number per group;

### A.4. Animals

Strain: BALB/c Nude
Age: 7-8 weeks (Treatment starting)
Gender: female
Total #: 32 mice plus spare

### A.5. Animal Housing

The mice will be housed in individual ventilated cages (4 per cage) at the following conditions:
   Temperature: 20-26° C.
   Humidity 30-70%
   Photoperiod: 12 hours light and 12 hours dark
   Polysulfone cage with size of 325 mm×210 mm×180 mm
   Bedding material is corn cob and changed weekly
   Diet: Animals will have free access to irradiation sterilized dry granule food during the entire study period.
   Water: Animal will have free access to sterile drinking water
   Cage identification label: number of animals, sex, strain, receiving date, treatment, study number, group number, and the starting date of the treatment
   Animal identification: Animals were marked by ear tag

### A.6. HuPrime® Model Profile

HuPrime® Liver cancer model L11097 derived from a male HCC patient was selected for this efficacy study. This model reached 1000 mm³ in 20-25 days post inoculation.

### A.7. Test and Positive Control Articles

Product identification: GamaMabs's Ab (3C23K)
Manufacturer: GamaMabs Pharma
Lot number: R18H2-LP01
Batch: 04GAM140513API
Quantity needed: 255 mg based on animal BW of 25 g with 50% spare
Package and storage condition: [30 ml/tube], 30 ml, [2-8° C.]
Concentration: 10.1 g/L
Product identification: Sorafenib
Manufacturer: Melonepharma
Lot number: D1111A
Quantity needed: 300 mg based on animal BW of 25 g with 50% spare
Package and storage condition: 400 mg, [RT]

### A.8. Experimental Methods and Procedures

### A.8.1. Tumor Inoculation and Group Distribution

Tumor fragments from stock mice inoculated with selected primary human cancer tissues were harvested and used for inoculation into BALB/c nude mice. Each mouse was inoculated subcutaneously at the right flank with primary human HCC model LI1097 fragment (R12P4, 2-4 mm in diameter) for tumor development on Jun. 9, 2015. The parent mouse number was #80150. #80151 and #80153. The mice were grouped when the average tumor size reached about 145 mm³ on Jun. 24, 2015. Mice were allocated randomly into 4 experimental groups according to their tumor sizes. Each group consisted of 8 mice, 4 mice per cage. The day was denoted as day 0. The test articles were administered to the tumor-bearing mice from day 0 (Jun. 24, 2015) through day 27 (Jul. 21, 2015) according to pre-determined regimen shown in Section 1.1 Experimental Design.

### A.8.2. Stop-dosing Regimen

When individual mouse has a body weight loss ≥20%, the mouse will be given dosing holiday(s) until its body weight recovers to the baseline. In this study, no dosing had been stopped.

### A.8.3. Observations

All the procedures related to animal handling, care, and the treatment in this study were performed according to guidelines approved by the Institutional Animal Care and Use Committee (IACUC) of CrownBio following the guidance of the Association for Assessment and Accreditation of Laboratory Animal Care (AAALAC). At the time of routine monitoring, the animals were checked for any effects of tumor growth on normal behavior such as mobility, food and water consumption (by looking only), body weight gain/loss, eye/hair matting and any other abnormal effect. Death and observed clinical signs were recorded on the basis of the numbers of animals within each subset.

### A.8.4. Tumor Measurements and the Endpoints

Tumor size were measured twice weekly in two dimensions using a caliper, and the volume is expressed in mm³ using the formula: TV=0.5 a×b², where a and b are the long and short diameters of the tumor, respectively. The tumor size is then used for calculations of TGI, T/C, and T-C values according to the description in the Table 2 in Abbreviations.

### A.8.5. Termination

The study was ended after 28 days treatment and mice were sacrificed.

Under following conditions, the in-life experiment of individual animal or whole groups will be terminated, by humane euthanization, prior to death, or before reaching a comatose state.
In a continuing deteriorating condition with severe clinical signs of severe distress and/or pain, inaccessible to adequate food or water;
Significant body mass (emaciated) (>20%);
Individual mouse with tumor size exceeding 3000 mm³ or MTV>2000 mm³.

### A.8.6. Statistical Analysis

Summary statistics, including mean and the standard error of the mean (SEM), are provided for the tumor volume of each group at each time point. Statistical analysis of difference in tumor volume among the groups was evaluated using a one-way ANOVA followed by multiple comparisons using Games-Howell. All data were analyzed using SPSS 16.0. P<0.05 was considered to be statistically significant.

### B. Results

### B.1. Body Weights

The results of body weights and body weight changes in the tumor bearing mice have bee measured. All the mice have completed their treatment without dosing holiday. No animal death or significant body weight loss has been observed in GamaMabs's Ab treated mice, but 7% body weight loss were observed in Sorafenib treated mice.

### B.2. Tumor Volumes

The tumor sizes of the different groups at different time points are shown in Table 7.

TABLE 7
Tumor Sizes in the Different Treatment Groups

| | Tumor Volume (mm³) | | | |
|---|---|---|---|---|
| | | GamaMabs's Ab, 20 | | |
| | Vehicle, | mg/kg, BIW × 4 | GamaMabs's Ab, 50 | Sorafenib, |
| Days | BIW × 2 weeks | weeks | mg/kg, BIW × 4 weeks | 50 mg/kg, QD × 4 weeks |
| 0 | 145.08 ± 17.70 | 145.15 ± 16.79 | 145.24 ± 16.38 | 145.18 ± 16.97 |
| 2 | 439.23 ± 54.14 | 358.57 ± 51.86 | 297.78 ± 46.32 | 321.35 ± 45.66 |
| 6 | 937.83 ± 99.91 | 665.09 ± 85.00 | 532.71 ± 104.17 | 493.84 ± 65.13 |
| 9 | 1556.55 ± 248.13 | 952.12 ± 171.45 | 751.81 ± 176.15 | 695.20 ± 66.81 |
| 13 | 2269.46 ± 356.55 | 1179.90 ± 232.26 | 1117.12 ± 302.85 | 891.50 ± 103.33 |
| 16 | | 1479.51 ± 292.49 | 1476.74 ± 407.93 | 1135.40 ± 133.62 |
| 20 | | 1973.13 ± 372.07 | 1602.61 ± 481.85 | 1478.84 ± 189.62 |
| 23 | | 1814.59 ± 231.17 | 1148.22 ± 381.49 | 1627.4 ± 202.91 |
| 27 | | 2081.67 ± 213.28 | 1454.47 ± 479.27 | 1829.66 ± 256.4 |

Note:
data expressed as Mean ± SEM.

### B.2. Tumor Growth Inhibition

The tumor growth inhibition is summarized in Table 8.

TABLE 8
Antitumor Activity of Test Compound GamaMabs's Ab and Sorafenib Treatment
in HuPrime ® Liver Xenograft Model LI1097

| | Tumor size | Tumor size | | | T-C | |
|---|---|---|---|---|---|---|
| | (mm³)^{a} | (mm³)^{a} | TGI | T/C | (days) at | |
| Treatment | Day 0 | Day 13 | (%) | (%) | 1000 mm³ | P value^{b} |
| G1 Vehicle | 145.08 ± 17.70 | 2269.46 ± 356.55 | — | — | — | — |
| G2 GamaMabs's | 145.15 ± 16.79 | 1179.90 ± 232.26 | 51.3% | 48.7% | 3 | 0.100 |
| Ab, 20 mg/kg | | | | | | |
| G3 GamaMabs's | 145.24 ± 16.38 | 1117.12 ± 302.85 | 54.3% | 45.7% | 5 | 0.111 |
| Ab, 50 mg/kg | | | | | | |
| G4 Sorafenib, | 145.18 ± 16.97 | 891.50 ± 103.33 | 64.9% | 35.1% | 8 | 0.024* |
| 50 mg/kg | | | | | | |

Note:
^{a}Mean ± SEM
^{b}Compared with the vehicle by multiple comparisons using Games-Howell.
*P < 0.05, compared with G1 Vehicle.

### B.3. Tumor Growth Curves

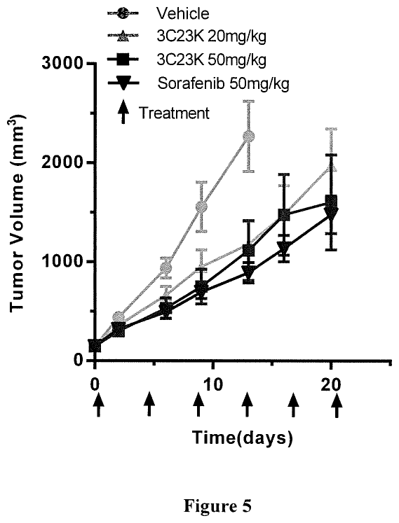

The tumor growth curves of different groups are shown in FIG. 5.

FIG. 5 represents the tumor Volumes of Mice in Different Groups during Test Compound GamaMabs's Ab and Sorafenib Treatment in HuPrime® Liver Xenograft Model LI1097

### B.4. Results Summary and Discussion

In this study, the efficacy of the test compound GamaMabs's Ab and positive control drug Sorafenib were evaluated in the treatment of HuPrime® HCC xenograft model LI1097 in female BALB/c nude mice.

In group 1 (Vehicle, BIW×2 weeks, i.v.), group 2 (GamaMabs's Ab 20 mg/kg, BIW×4 weeks, i.v.), group 3 (GamaMabs's Ab 50 mg/kg, BIW×4 weeks, i.v.) and group 4 (Sorafenib, 50 mg/kg, QD×4 weeks, p.o.), the body weight change at study termination was 0.67%, 2.68%-0.38% and −7.63%, respectively. The test compound GamaMabs's Ab at 20 mg/kg and 50 mg/kg were well tolerated in the LI1097 tumor-bearing mice. The mice in the Sorafenib 50 mg/kg treated group exhibited mean maximum body weight loss of 7.63% on day 27 of treatment.

The mean tumor size of the vehicle treated mice reached 2269.46 mm³ on day 13. Group 2 (GamaMabs's Ab, 20 mg/kg) and group 3 (GamaMabs's Ab, 50 mg/kg) produced 50% anti-tumor response vs vehicle treatment with TGI of 51.3% and 54.3% (P=0.100 and 0.111) respectively. Group 4 (Sorafenib, 50 mg/kg) produced significant anti-tumor activity with TGI of 64.9% on day 13 of treatment (P=0.024). The results of tumor sizes in different groups at different time points after treatments presented in the Table 8 and FIG. 5 show that responses to treatment in groups 2 and 3 (GamaMab's AB, 20 and 50 mg/kg respectively) are maintained, as with sorafenib, for at least 27 days. However, tumor responses in group 2 and 3 are probably too heterogeneous for obtaining a better statistical significance.

In summary, in this study, the test compound GamaMabs's Ab produced an anti-tumor activity against the primary HuPrime® HCC xenograft model LI1097 close to that induced by sorafenib, the standard of care for this pathology. Moreover, anti-tumor activity of GM102 was not accompanied by any toxic event whilst sorafenib treatment induced up to 7% of mean body weight loss.

### Example 5: In Vivo Efficacy of Anti-AMHRII Immunoconjugates Against AMHRII-Expressing Non-Gynecologic Cancers

### A. Materials and Methods

### A.1. Abbreviations

Commonly used abbreviations in this example are the same as those of Table 3 and Table 4 of Example 4.

### A.2. Objective

To evaluate preclinically the in vivo efficacy of GamaMabs's compound GM103 in the treatment of PDX model LI1097 in female BALB/c nude mice.

### A.3. Experimental Design

TABLE 9
Study design of efficacy study

| | | | Dose | Dosing | | |
|---|---|---|---|---|---|---|
| | | | Level | Volume | | |
| Group | N | Treatment | (mg/kg) | (ml/kg) | Route | Schedule |
| 1 | 8 | Vehicle | — | 10 | IV | One single dose |
| 2 | 8 | GM103 | 1 | 10 | IV | One single dose |
| 3 | 8 | GM103 | 5 | 10 | IV | One single dose |
| 4 | 8 | GM103 | 10 | 10 | IV | One single dose |

Note:
N: animal number per group

### A.4. Materials

### A.4.1. Animals

Strain: BALB/c nude
Age: 6-8 weeks
Gender: Female
Total #: 32 mice plus spare

### A.4.2. Animal Housing

The mice will be housed in individual ventilated cages (4-5 mice per cage) at the following conditions:
Temperature: 20˜26° C.
Humidity 30-70%
Photoperiod: 12 hours light and 12 hours dark
Polysulfone cage with size of 325 mm×210 mm×180 mm
Bedding material is corn cob and changed weekly
Diet: Animals will have free access to irradiation sterilized dry granule food during the entire study period.
Water: animal will have free access to sterile drinking water
Cage identification label: number of animals, gender, strain, receiving date, treatment, —Project ID, group number, animal ID and the starting date of the treatment
Animal identification: Animals were marked by ear Tag

### A.4.3. Model Info

HuPrime® liver cancer xenograft model LI1097 was selected for this efficacy study.

### A.4.4. Test and Control Articles

Product identification: GM103
Manufacturer: GamaMabs Pharma
Physical description: solution
Batch number: GAM100-NC005-4
Quantity needed: 4.48 mg based on animal BW of 25 g with 40% spare
Package and storage condition: 4.3 mg/1.3 ml/vial, stored at 4° C.

### A.5. Experimental Methods

### A.5.1. Tumor Inoculation

Each mouse will be inoculated subcutaneously at the right flank with primary human liver cancer xenograft model LI1097 fragment (2-3 mm in diameter) for tumor development.

### A.5.2. Group Assignment

When average tumor size reaches approximately 200 mm³, mice will be randomly allocated into 4 groups shown in Table 3. Each group contains 8 mice.

### A.5.3. Testing Article Dosing Solution Preparation

Volume type: Adjust dosing volume for body weight (Dosing volume=10 μL/g)

TABLE 10
Detailed instructions on formulation and storage

| | Dose | | Con- | |
|---|---|---|---|---|
| | (mg/ | | centration | |
| Compounds | kg) | Preparation | (mg/ml) | Storage |
| GM103 (1) | 1 | Dilute 0.073 ml GM103 stock | 0.1 | Prepare |
| | | solution (3.308 mg/ml) with | | fresh |
| | | 2.327 ml saline or PBS?. | | |
| GM103 (2) | 5 | Dilute 0.363 ml GM103 stock | 0.5 | Prepare |
| | | solution (3.308 mg/ml) with | | fresh |
| | | 2.037 ml saline or PBS?. | | |
| GM103 (3) | 10 | Dilute 0.726 ml GM103 stock | 1 | Prepare |
| | | solution (3.308 mg/ml) with | | fresh |
| | | 1.674 ml saline or PBS?. | | |

### A.5.4. Observation

After tumor inoculation, the animals will be checked daily for morbidity and mortality. At the time of routine monitoring, the animals will be checked for any effects of tumor growth and treatments on normal behavior such as mobility, food and water consumption, body weight gain/loss, eye/hair matting and any other abnormal effect. Death and observed clinical signs will be recorded on the basis of the numbers of animals within each subset.

Tumor size will be measured by caliper twice weekly in two dimensions. The tumor volume will be expressed in mm³ using the formula: TV=0.5 a×b² where a and b are the long and short diameters of the tumor, respectively.

Body weight will be measured twice weekly.

### A.5.5. End Points

Following analysis will be applied at the endpoint: TGI(Tumor Growth Index) and TC.

### A.5.6. Termination

Under following conditions, the in-life experiment of individual animal or whole group will be terminated, by humane euthanization, prior to death, or before reaching a comatose state.
In a continuing deteriorating condition with severe clinical signs of severe distress and/or pain, inaccessible to adequate food or water;
Significant body mass loss (emaciated) (>20%);
Individual mouse with tumor size exceeding 3000 mm³ or whole group of mice with MTV>2000 mm³.

### A.5.7. Statistics Analysis

For comparison among three or more groups, a one-way ANOVA will be performed followed by multiple comparison procedures. All data will be analyzed using SPSS 16.0. P<0.05 is considered to be statistically significant.

### A.6. Compliance

The protocol and any amendment(s) or procedures involving the care and use of animals in this study will be reviewed and approved by the Institutional Animal Care and Use Committee (IACUC) of CrownBio prior to conduct. During the study, the care and use of animals will be conducted in accordance with the regulations of the Association for Assessment and Accreditation of Laboratory Animal Care (AAALAC).

### B. Results

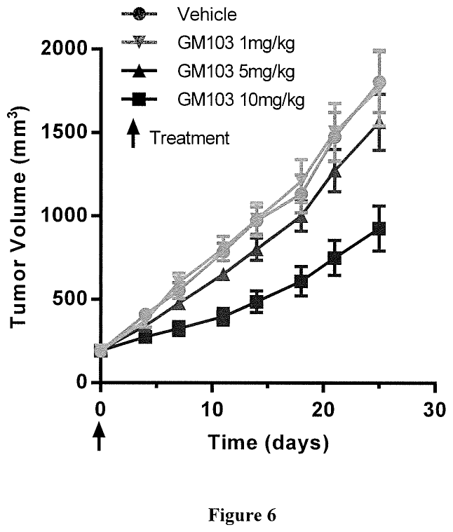

The results of FIG. 6 showed the in vivo anti-cancer activity of the GM103 ADC immunoconjugate at a dose of 5 mg/kg or more.

### Example 6: AMHRII Expression in Further Non-Gynecologic Cancers

### A. Materials and Methods

### A.1. AMHRII Membrane Expression Analysis by Flow Cytometry

### Preparation of Cells for Analysis

Tissues were dissected within 1 h of surgery, minced into 1-mm2 fragments and washed in RPMI containing penicillin (10%), streptomycin (10%) and gentamycin (0.1 mg/mL; Sigma-Aldrich).
Tissue fragments were digested for 2-4 h with collagenase and DNAse (2 mg/mL; Sigma-Aldrich) with rapid shaking at 37 C.
Mucus and large debris were removed by filtration through a 40-lm cell strainer.
Viable cells were obtained by Ficoll gradient centrifugation.

The quantitation of AMHRII binding sites on resuspended tumor cells was performed using The Quantum™ Simply Cellular (Bangs Laboratory) according to the manufacturer's instructions:
Briefly, the four microbeads populations labeled with a different calibrated amount of mouse anti-human IgG specific for the Fc portion of human IgG antibodies were stained with the AlexaFluor488-conjugated anti-AMHRII 3C23K. In FACS tubes, one drop of each vial in the kit is added to 50 μl of PBS 1×:
   1—Beads B (blank)
   2—Beads 1+3C23K-AF 10 μg/mL
   3—Beads 2+3C23K-AF 10 μg/mL
   4—Beads 3+3C23K-AF 10 μg/mL
   5—Beads 4+3C23K-AF 10 μg/mL (the concentration could be increased to 25 μg/ml if necessary)
Each bead population binds varying amounts of the AlexaFluor488-conjugated anti-AMHRII 3C23K, producing a corresponding intensity of fluorescence, which is analyzed on a FACS Canto II cytometer (BD).
A calibration curve was generated by plotting the mean fluorescence intensity of each bead population versus its assigned Antibody Binding Capacity (ABC).

Cells were usually stained in eppendorf tubes 1.5 ml.
All centifugation steps were done at 4° C.
All incubation steps were done at 4° C. to avoid antibody internalization.
3.5 Million Cells (trypsinized COV434-MISRII or freshly dissociated tumor cells) were centrifuged at 200-300 g for 5 min and were washed one time with PBS (500 μl per tube)
Wash with ice cold PBS/2% FBS (200-300 g for 3 min) and resuspend in 700 μl of PBS 1× and distribute 100 μl by FACS tube for the conditions described in Table 11 below:

TABLE 11

| COV434-MISRII | Fresh tumor cells |
|---|---|
| No antibody | |
| R565-AF (isotype control) 10 μg/mL | |
| 3C23K-AF 1 ng/mL | |
| 3C23K-AF 10 ng/mL | |
| 3C23K-AF 100 ng/mL | |
| 3C23K-AF 1 μg/mL | |

3C23K-AF 10 μg/mL (and up to 25 μg/ml when necessary)

Incubate with antibody 3C23K-AF488 in PBS/1% FBS for 30 min at 4° C.
Wash in PBS/2% BSA two times (200-300 g for 3 min)
Wash in PBS two times (200-300 g for 3 min)
Add 300-400 μl PBS and analyze on FACS as soon as possible
This protocol does not comprise any fixation step for extracellular staining to maintain the integrity of the membrane. Consequently, only membrane AMHRII is detected

### A.2. AMHRII Membrane Expression by Immunofluorescence

A method of indirect immunofluorescence was therefore developed with the anti-AMHRII 3C23K antibody conjugated to Alexa Fluor® 488. Signal amplification was then performed in two-steps with a rabbit anti-AF488 antibody and a goat anti-rabbit antibody conjugated to Alexa Fluor® 647.

Frozen tissue sections are made with the cryostat Leica CMD1950 keep at −20° C. Frozen tissue are mounted on metal disc with OCT compound and once solidified they were mounted on the disc holder. Section of 7 μm were realized and were put on the Superfrost Plus slides (Menzel Gläser) and immediately store at −20° C.

The frozen section slides were rehydrated with PBS 1× and then fixed 10 min at −20° C. by covering them with 300 μl of cold acetone (VWR Prolabo) and recovered with parafilm to ensure that all the tissue was totally recovered by the solution. After rising with PBS, slides were treated with 300 μl of blocking buffer (PBS1×-BSA2%-Goat serum10%-Triton X100 0.1%) 1 hour in a humidified box at RT to block unspecific interactions between antibodies and tissue components. The 3C23K-AF488 or isotype control R565-AF488 diluted at 10 μg/ml in blocking buffer were applied for 30 min at RT in the humidified box. After 3 washes with PBS1×-Triton X100 0.1% (3×10 min), antibody anti-AF488 (Invitrogen) diluted at 1/500 in blocking buffer were added (300 μl) for 30 min of incubation at RT. After 3 washes with PBS1×-Triton X100 0.1% (3×10 min), anti-rabbit antibody AF647conjugated (Invitrogen) diluted at 1/500 in blocking buffer were added (300 μl) for 30 min of incubation at RT. Washes (3×10 min) with PBS1×-Triton X100 0.1% were realized, then DAPI (Sigma-Aldrich) at 0.5 μg/ml were applied for 10 min. After rising with PBS and H₂O the slides sections were mounted under coverslips (24×50 mm, Knittel Glass) with a drop (50 μl) of DAKO Fluorescent mouting medium avoiding bubble air and store at 4° C. in the dark until they were imaged. Images acquisition were performed using fluorescence microscope Leica DM5000B equipped with the CoolSnap EZ CCD camera controlled by the Metavue software (Molecular Devices). Images post-treatments are performed using the ImageJ free software (http://imagej.nih.gov/ij/).

### B. Results

### B.1. AMHRII Expression in Fresh Human Colorectal Samples

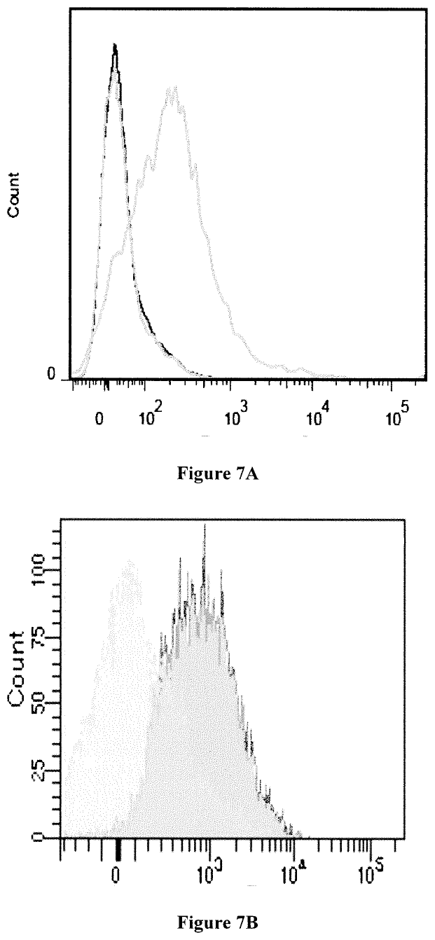

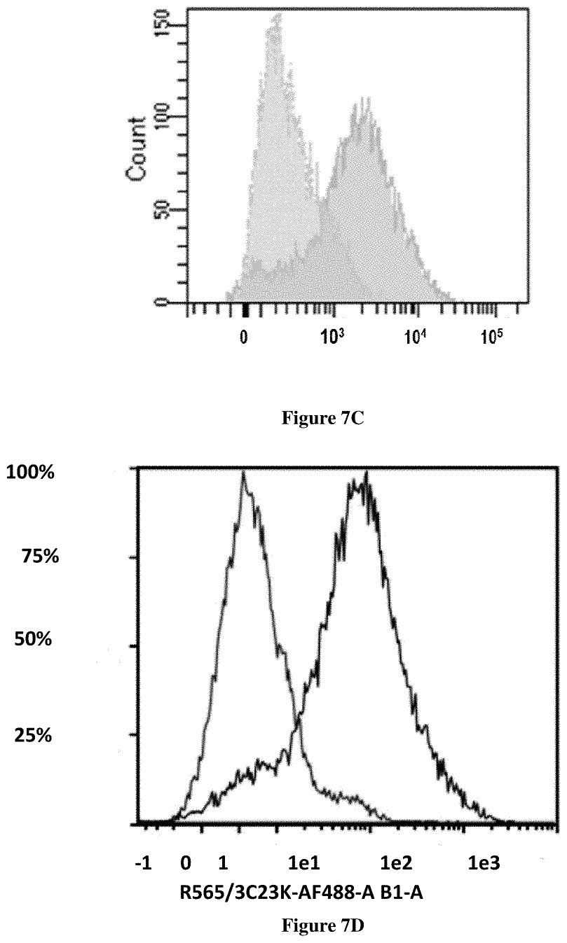

The FACS analysis of AMHRII membrane expression from tumor samples previously collected from four distinct individuals affected with a colorectal carcinoma are depicted in FIGS. 7A, 7B, 7C and 7D. The results show that the tumor cells (CD3-Epcam+) contained in the tumor samples express AMHRII at their membrane.

The results from tumor samples previously collected from 20 distinct individuals affected with a colorectal carcinoma are presented in Table 12.

In Table 12, AMHRII expression was assessed, in each tumor sample, by (i) determining the mean number of AMHRII proteins present at the tumor cell membrane and by (ii) determining the percentage of membranous AMHRII positive cells in the tumor sample. Indication of whether the corresponding tumor sample is set to be “positive” or “negative” is presented in the left column of Table 12. Indication “positive” means that AMHRII is significantly expressed at the tumor cell membrane. Indication “negative” means that AMHRII expression at the cell membrane is not significantly detected.

The results of Table 12 show 15 out of 20 tumor samples expressed membranous AMHRII, albeit at various expression levels.

Depending on the tumor samples, the mean number of membranous AMHRII proteins per tumor cell (termed “number of receptors per cell (tumor)” in Table 12) varied from 540 to more than 155 000.

Depending on the tumor samples, the frequency of membranous AMHRII protein expressing cells (termed “Percentage of AMHRII positive cells (Epcam+)” in Table 12) varied from 20% to 100%

The results of Table 12 did not show a correlation between the mean number of membranous AMHRII per tumor cell and the frequency of tumor cell expressing membranous AMHRII.

### B.2. AMHRII Expression in Human Colorectal Tumor Xenografts (Patient Derived Xenografts)

Human tumor xenografts samples were obtained as disclosed in Example 3 and AMHRII expression by the tumor cells was assessed using the methods disclosed in the Materials and Methods section.

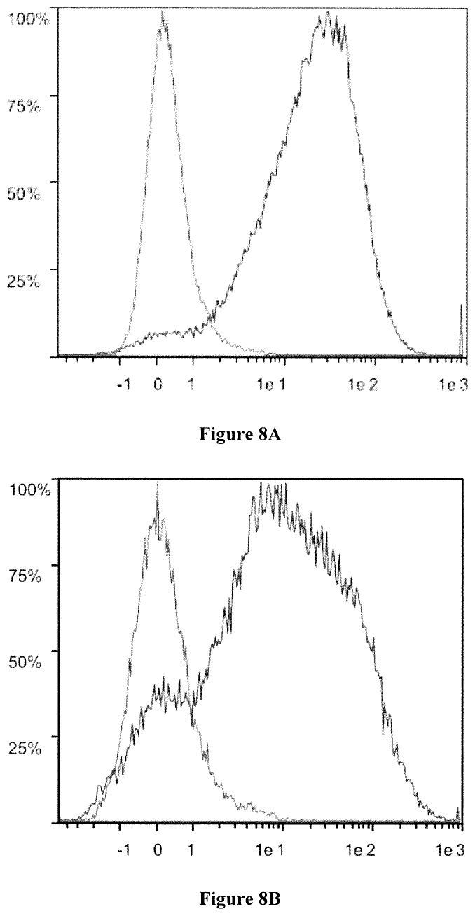

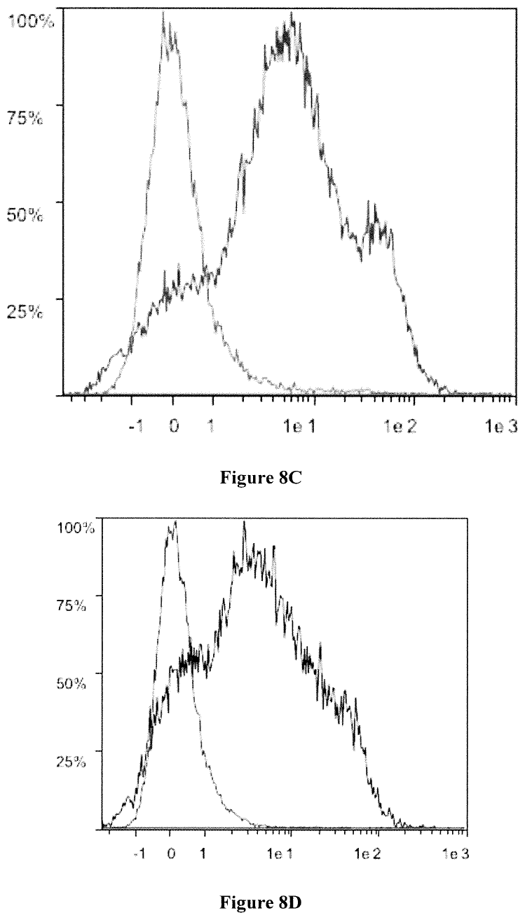

The FACS analysis of AMHRII membrane expression from tumor samples previously collected from four distinct individuals affected with a colorectal carcinoma and then xenografted in mice are depicted in FIGS. 8A, 8B, 8C and 8D. The results show that the tumor cells (CD3-Epcam+) contained in the xenografted tumor samples express AMHRII at their membrane.

The results from tumor samples previously collected from 12 distinct individuals affected with a colorectal carcinoma, and then xenografted in mice are presented in Table 13.

In Table 13, AMHRII expression was assessed, in each xenograft tumor sample, by (i) determining the mean number of AMHRII proteins present at the tumor cell membrane and by (ii) determining the percentage of membranous AMHRII positive cells in the xenograft tumor sample.

The results of Table 13 show that 6 out of 12 xenograft tumor samples expressed membranous AMHRII, albeit at various expression levels.

Depending on the xenograft tumor samples, the mean number of membranous AMHRII proteins per cell (termed “number of receptors per cell (Epcam+)” in Table 13) varied from more than 16 000 to about 100 000.

Depending on the tumor samples, the frequency of membranous AMHRII protein expressing cells (termed “Percentage of AMHRII positive cells (Epcam+)” in Table 13) varied from 0.5% to 87%.

The results of Table 13 did not show a clear correlation between the mean number of membranous AMHRII per tumor cell and the frequency of tumor cell expressing membranous AMHRII.

Indication of whether the corresponding tumor sample is set to be “positive” or “negative” is presented in the left column of Table 13. Indication “positive” means that AMHRII is not significantly expressed at the membrane of tumor cells. Indication “negative” means that membrane AMHRII expression by the tumor cells is not significantly detected.

### B.3. AMHRII Membrane Expression in Fresh Renal Cell Carcinoma Samples

Human renal cell carcinoma tumor samples were obtained with the methods disclosed in the Materials and Methods section and membrane AMHRII expression by the tumor cells (EpCam+) has been assessed by FACS analysis.

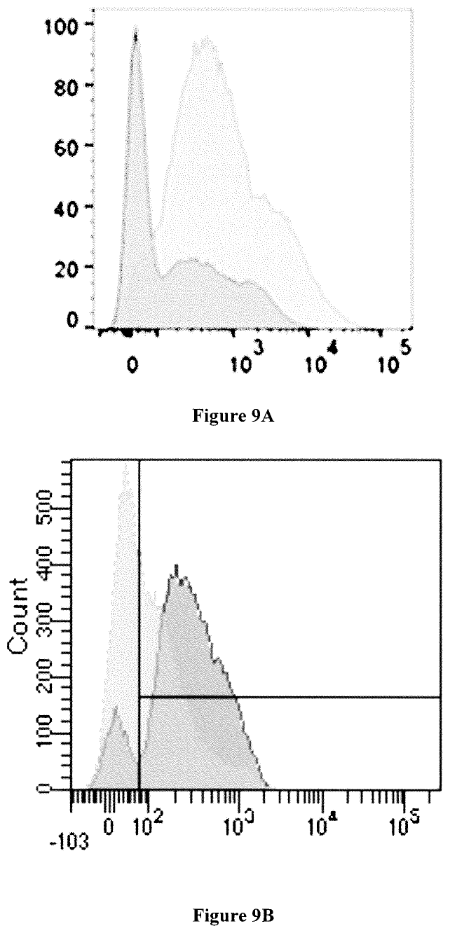

The results are depicted in FIGS. 9A and 9B.

The FACS analysis of AMHRII membrane expression from tumor samples previously collected from two distinct individuals affected with a renal cell carcinoma are depicted in FIGS. 9A and 9B. The results show that the tumor cells (CD3-Epcam+) contained in the renal cell carcinoma tumor samples express AMHRII at their membrane.

### Example 7: In Vivo Efficacy of Anti-AMHRII Antibodies Against AMHRII-Expressing Non-Gynecologic Cancers

### A. Materials and Methods

Stock mice (Athymic Nude-Foxn1^{nu} from Envigo) were implanted with tumor fragments from Champions TumorGraft® model CTG-0401. After the tumors reached 1000-1500 mm³, they were harvested and the tumor fragments were implanted SC in the left flank of the female study mice. Each animal was implanted with a specific passage lot: passage 6 for CTG-0401. Tumor growth was monitored twice a week using digital calipers and the tumor volume (TV) was calculated using the formula (0.52×[length×width²]). After the tumor volume reached 175±7 mm³, mice were selected based on their tumor size and were randomly allocated into 4 groups of 12 animals per group (Day 0). After the initiation of dosing on Day 0, animals were weighed twice per week using a digital scale and TV was measured twice per week and also on the final day of study. The study was terminated when the mean tumor volume in the vehicle control group reached 1500 mm³ or up to Day 60, whichever occurred first. The study design is summarized in Table 14 below.

TABLE 14
Design of Efficacy Study in Model
CTG-0401 of Human Colorectal Cancer

| | | | | Dose | | | Total |
|---|---|---|---|---|---|---|---|
| | | | Dose | Volume | | Dosing | # of |
| Group | n | Agent | (mg/kg) | (mL/kg) | Route | Schedule | Doses |
| | | Vehicle | 0 | 10 | IP | BIWx4 | 8 |
| | | GM102 | | | | | |
| 1 | 12 | Vehicle | 0 | 10 | IP | Q7Dx3 | 3 |
| | | Irinotecan | | | | | |
| 2 | 12 | GM102 | 20 | 10 | IP | BIWx4 | 8 |
| 3 | 12 | Irinotecan | 100 | 10 | IP | Q7Dx3 | 3 |

GM102 or GM102 vehicle was administered before Irinotecan or Irinotecan vehicle.

### B Results

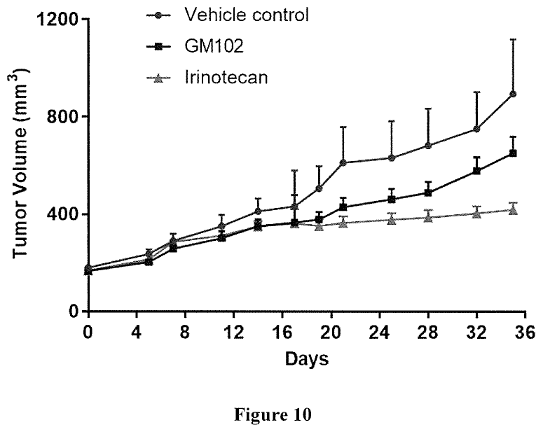

The results of this experiment are depicted in FIG. 10.

The results of FIG. 10 show that the anti-AMHRII antibody GM102 possesses an efficient in vivo anti-tumor effect against an AMHRII-expressing human colorectal tumor.

Noticeably, the anti-AMHRII antibody GM102 exerts an anti-tumor effect which is indistinguishable from the anti-tumor effect of the mainly used anti-colon cancer molecule Irinotecan (CAS number: 100286-90-6).

### Example 8: In Vivo Efficacy of a Combined Treatment with (i) Anti-AMHRII Antibodies and (ii) a Combination of Trifluridine and Tipiracil Against AMHRII-Expressing Non-Gynecologic Cancers

### A. Materials and Methods

### A.1. Cohort of Patients

This example presents the results of a phase IIa clinical study of a combined treatment of patients affected with metastatic colorectal cancer (mCRC) with a combination of (i) the 3C23K anti-AMHRII monoclonal antibody (also termed “GM102” herein) and (ii) the pharmaceutical specialty termed “Lonsurf®” which consists of a combination of trifluridine and tipiracil.

Each dose of Lonsurf comprises 20 mg trifluridine and 8.19 mg tipiracil.

The cohort of mCRC patients comprised 15 evaluable patients who have previously received at least two prior lines of standard chemotherapy against mCRC. These patients were eligible for a treatment with Lonsurf® and have failed or have not being considered as candidate patients for a chemotherapy treatment with fluoropyrimidines, oxaliplatin, irinotecan, anti-VEGF agents, regorafenib and anti-EGFR agents.

### A.2. Level of Expression of AMHRII

The expression level of AMHRII was determined on biopsies of tumor tissue samples collected from the patients belonging to the studied cohort. The level of expression of AMHRII by the cells contained in the biopsy tumor tissue samples, both (i) as the percentage of positive cells and (ii) as a measure of the membranous score, was measured as described in the Materials and Methods of Example 1 (See § A.2. of the Materials and Methods section of Example 1).

### A.3. Treatment Schedule

Each enrolled patient of the treated cohort was administered the combined treatment at the following amount of active ingredients per dose: (i) 3C23K anti-AMHRII antibody (GM102) at 7 mg/kg and (ii) Lonsurf® at 35 mg/m².

Each enrolled patient was administered a treatment dose (See above) according to monthly treatment cycles, with each 28 days treatment cycle comprising (i) a weekly administration of GM102 between Day 1 and Day 15 and (ii) a twice daily administration of Lonsurf® from Day 1 to Day 5 and then from Day 8 and D12.

### B. Results

### B.1. Clinical Outcome

The fifteen enrolled mCRC patients received the prescribed combined treatment of the 3C23K anti-AMHRII antibody and Lonsurf® for various period of times.

For patient “04-01”, treatment with GM102 was permanently discontinued at the end of Cycle 2. For patient “02-01”, treatment with GM102 was permanently discontinued at the end of Cycle 2. For patient “02-01”, treatment is pursued beyond Cycle 8. For patient “02-07”, treatment with GM102 was permanently discontinued at the end of Cycle 2. For patient “02-09”; treatment with GM102 was permanently discontinued at the end of Cycle 2. For patient 05-05”, treatment with GM102 was permanently discontinued at the end of Cycle 2. For patient “05-06”, treatment with GM102 was permanently discontinued at the end of Cycle 2. For patient “01-06”, treatment is pursued beyond Cycle 6. For “patient” 01-07”, treatment with GM102 was permanently discontinued at the end of Cycle 2. For patient “05-08”, treatment is pursued beyond Cycle 6. For patient “04-05”, treatment with GM102 was permanently discontinued at the end of Cycle 4. For patient “05-09”, treatment with GM102 was permanently discontinued at the end of Cycle 2. For patient “01-13”, treatment is pursued until completion of Cycle 3. For patient “05-13”, treatment is pursued until completion of Cycle 3. For patient “02-12”, treatment is pursued until completion of Cycle 3.

The activity data per investigator's assessment are presented in Table 15 below.

TABLE 15
Activity data

| | | Cycle | Cycle |
|---|---|---|---|
| Patients | Cycle 2, Day 28 | 4, Day 28 | 6, Day 28 |
| 04-01 | PD^{a} (+25%) | — | — |
| 02-01 | PD (+65%) | — | — |
| 02-06 | SD^{b}(−3%) | SD (+7% from | SD |
| | | NADIR) | |
| 02-07 | PD (+21%) | | |
| 02-09 | SD (+15%) | SD (+0%) | SD |
| 05-05 | SD (+8%) clinical | — | — |
| | progression | | |
| 05-06 | PD (brain metastasis) | — | — |
| 01-06 | SD (0%) | SD (−6%) | ongoing |
| 01-07 | PD (+28%) | — | — |
| 05-08 | SD (0%) | SD (+9%) | ongoing |
| 04-05 | PD (+27%) | — | — |
| 05-09 | PD (+30%) | — | — |
| 01-13 | SD(+3%) | ongoing | |
| 05-13 | SD (+9%) | ongoing | |
| 02-12 | SD | ongoing | |

^{a}PD: Disease progression.
^{b}SD: Stable disease.

According to the clinical results, the number of evaluable patients stabilized is as defined hereafter:
at the end of Cycle 2 of treatment: 8/15 patients (53%),
at the end of Cycle 4 of treatment: 4/12 (33%), and
at the end of Cycle 6 of the treatment: 2/10 (20%)
   B.2. Analysis of the Clinical Outcome in Relation with AMHRII Expression

Confirmation of a relevant relationship between (i) the level of AMHRII expression by cells contained in the tumor tissue samples and (ii) efficacy of the combined treatment with GM102 and Lonsurf® has been shown.

The level of AMHRII expression in tumor samples from patients having undergone four treatment cycles was compared with the level of AMHRII expression in tumor samples from patients having undergone only two treatment cycles.

Comparison was made between (i) “Stab+” patients treated for at least four treatment cycles and “Stab−” patients treated for a maximum of two treatment cycles.

The results are depicted in FIG. 11 (percent AMHRII positive cells) and FIG. 12 (AMHRII membranous score values).

In FIG. 11, “Stab+” patients have a mean of 25% of AMHRII-positive cells whereas “Stab−” patients have a mean of 10.1% AMHRII-positive cells.

In FIG. 12, “Stab+” patients have a mean of 55.8 of AMHRII-membranous score whereas “Stab−” patients have a mean of 12.3 AMHRII-membranous score.

These results show that patients treated for at least four treatment cycles (“Stab+”, n=6) had a higher AMHRII expression level that the patients which have been treated for a maximum of two treatment cycles (“Stab−”, n=7).

Further, it is shown that the duration of the combined treatment with the 3C23K anti-AMHRII antibody and Lonsurf® correlates with the AMHRII membranous score, as it is depicted in FIG. 13.

According to the correlation results, the Spearman r test value was of 0.542, the 95% confidence interval ranged from −0.055595 to 0.8397 and the P (one-tailed) value was of 0.0342, using 13 XY pairs.

As shown in FIG. 14, it has been shown that patients having a AMHRII membranous score of 20 or more benefit from a longer treatment duration, as compared to patients having a AMHRII membranous score lower than 20. This difference in treatment duration is expected to increase with time, since the patients having a AMHRII membranous score of 20 or more are still under treatment with the combined therapy of the 3C23K anti-AMHRII antibody (GM102) and Lonsurf®.

TABLE 12
AMHRII expression in fresh human colorectal tumor samples

| | | | Number of | Percentage of AMHRII | |
|---|---|---|---|---|---|
| | | | receptors per cell | positive cells | |
| sample | Id | Histological type | (tumor) | (Epcam+) | Positive/negative |
| #1 | C1 | AdenoK | 15.600 | 100% | + |
| #2 | I1 | AdenoK | 155.954 | 20% | + |
| #3 | E1 | AdenoK | 23.548 | 100% | + |
| #4 | A2 | AdenoK | 12.680 | 26% | + |
| #5 | N1 | AdenoK (left colon) | 116.704 | 50% | + |
| #6 | N2 | AdenoK (left colon) | 7.578 | | − |
| #7 | N3 | AdenoK (right colon) | 34.677 | 100% | + |
| #8 | N4 | AdenoK (left colon) MSI | 1.605 | | − |
| #9 | A1 | MucinousAdenoK(sigmoid) | 540 | | − |
| #10 | E2 | AdenoK | 57.209 | 100% | + |
| #11 | I2 | AdenoK | 155.473 | 27% | + |
| #12 | I3 | AdenoK | 102.275 | 68% | + |
| #13 | N6 | AdenoK (left colon) | 47.464 | 100% | + |
| #14 | N7 | AdenoK (left colon) | 61.870 | 100% | + |
| #15 | E3 | AdenoK | 4.090 | | − |
| #16 | E4 | AdenoK | 32.153 | 75% | + |
| #17 | A3 | AdenoK (sigmoid) | 6.400 | | − |
| #18 | E5 | AdenoK | 13.152 | 37% | + |
| #19 | E6 | AdenoK | 21.962 | 25% | + |
| #20 | A4 | AdenoK | 42.596 | 56% | + |

TABLE 13
AMHRII in tumor cells from xenografted human tumors

| | | Histological | Nb of receptors per | Percentage of AMHRII | |
|---|---|---|---|---|---|
| Reference | Number | type | cell (Epcam+) | positive cells (Epcam+) | Positive/Negative |
| CO14452B | #1 | Muc | 63.181 | 16% | + |
| | | adenoK | | | |
| CO14744C | #2 | AdenoK | 25.269 | 1.5% | − |
| CO13196D | #3 | AdenoK | 21.313 | 4% | − |
| CO11291 | #4 | AdenoK | 20.629 | 0.5% | − |
| CO10619 | #5 | AdenoK | 16.327 | 0.5% | − |
| CO11690 | #6 | AdenoK | 17.802 | 1% | − |
| CO10069 | #7 | AdenoK | 44.511 | 2% | − |
| CO14592 | #8 | AdenoK | 83.762 | 87% | + |
| CO10708 | #9 | AdenoK | 43.109 | 7% | + |
| CO7935 | #10 | AdenoK | 99.959 | 73% | + |
| CO11101 | #11 | AdenoK | 28.951 | 44% | + |
| CO10748 | #12 | AdenoK | 29.821 | 56% | + |

1. A method for treating a non-gynecologic cancer in an individual in need thereof comprising administering a human AMHRII-binding agent to the individual.

2. The method according to claim 1, wherein the non-gynecologic cancer is selected from the group consisting of colon cancer, liver cancer, hepatocellular carcinoma, testis cancer, thyroid cancer, gastric cancer, gastrointestinal cancer, bladder cancer, pancreatic cancer, head and neck cancer, kidney cancer, liposarcoma, fibrosarcoma, pleuramesothelioma, melanoma, sarcoma, brain cancer, osteocarcinoma, breast cancer, prostate cancer and leukemia.

3. The method according to claim 1, wherein the human AMHRII-binding agent is a monoclonal anti-AMHRII antibody or an AMHRII-binding fragment thereof.

4. The method according to claim 1, wherein the human AMHRII-binding agent is a monoclonal antibody selected from the group consisting of:
a) a light chain comprising SEQ ID NO: 2 and a heavy chain comprising SEQ ID NO: 4 (3C23 VL and VH sequences without leaders);
b) a light chain comprising SEQ ID NO: 6 and a heavy chain comprising SEQ ID NO: 8 (3C23K VL and VH sequences without leaders);
c) a light chain comprising SEQ ID NO: 10 and a heavy chain comprising SEQ ID NO: 12 (3C23 light and heavy chains without leaders);
d) a light chain comprising SEQ ID NO: 14 and a heavy chain comprising SEQ ID NO: 16 (3C23K light and heavy chains without leaders).

5. The method according to claim 1, wherein the human AMHRII binding agent is a monoclonal antibody comprising CDRs comprising the following sequences:
CDRL-1: RASX1X2VX3X4X5A (SEQ ID NO. 65), where X1 and X2 are, independently, S or P, X3 is R or W or G, X4 is T or D, and X5 is I or T;
CDRL-2 is PTSSLX6S (SEQ ID NO. 66) where X6 is K or E; and
CDRL-3 is LQWSSYPWT (SEQ ID NO. 67);
CDRH-1 is KASGYX7FTX8X9HIH (SEQ ID NO. 68) where X7 is S or T, X8 is S or G and X9 is Y or N;
CDRH-2 is WIYPX10DDSTKYSQKFQG (SEQ ID NO. 69) where X10 is G or E; and
CDRH-3 is GDRFAY (SEQ ID NO. 70)

6. The method according to claim 1, wherein the AMHRII binding agent consists of an Antibody Drug Conjugate (ADC).

7. The method according to claim 1, wherein the human AMHRII binding agent is an AMHRII-binding engineered receptor.

8. The method according to claim 1, wherein the AMHRII binding agent is a cell expressing an AMHRII-binding engineered receptor.

9. The method according to claim 8, wherein the cell expressing an AMHRII-binding engineered receptor is a CAR T-cell or a NK T-cell.

10. The method according to claim 1, wherein the human AMHRII binding agent is used in combination with another anti-cancer treatment.

11. A method of treating a colorectal cancer in an individual in need thereof, comprising the steps of:
a) administering an anti-AMHRII antibody,
b) administering trifluridin, optionally combined with a thymidine phosphorylase inhibitor such as tipiracil,
wherein administering steps a) and b) are performed simultaneously or sequentially.

12. A method of treating a colorectal cancer in an individual in need thereof, comprising the steps of:
a) administering an anti-AMHRII antibody,
b) administering trifluridin combined with a thymidine phosphorylase inhibitor such as tipiracil,
wherein administering steps a) and b) are performed sequentially.

13. A method of treating a colorectal cancer cancer in an individual in need thereof, comprising performing one or more monthly cycles of treatment, wherein each monthly cycle of treatment comprises the steps of:
a) weekly administering an anti-AMHRII antibody from Day 1 to Day 15 of the monthly cycle of treatment,
b) twice daily administering trifluridin combined with a thymidine phosphorylase inhibitor such as tipiracil, from Day 1 to Day 5 and then from Day 8 to Day 12 of the monthly cycle of treatment,
wherein administering steps a) and b) are reiterated during the selected number of monthly cycles of treatment.

14. The method according to claim 11, wherein the monthly cycle of treatment consists of a 28 days cycle of treatment.

15. The method according to claim 12, wherein the monthly cycle of treatment consists of a 28 days cycle of treatment.

16. The method according to claim 13, wherein the monthly cycle of treatment consists of a 28 days cycle of treatment.

17. A method for determining whether an individual is responsive to a cancer treatment with an AMHRII-binding agent, wherein the method comprises the step of determining whether a tumor tissue sample previously obtained from the said individual expresses the AMHRII protein at the cell surface, wherein if the tumor tissue sample expresses the AMHRII protein at the cell surface, then the individual is responsive.


SEQUENCE LISTING (74 sequences)

SEQ ID NO: 1 - DNA, 318 bp, Artificial Sequence
  Synthetic 3C_23 VL without leader
  Synthetic 3C_23 VL without leader
  Synthetic 3C_23 VL without leader
  CDS 1..318
gacatccagatgacacagtccccatctaccctgtctgcttccgtgggagatcgggtgactatcacctgcagagcaagctcctccgtgaggtacatcgcttggtaccagcagaagccaggaaaggccccaaagctgctgacctacccaacctcctccctggaatccggggtgcccagcagattctcaggcagtggctccggcaccgaattcaccctgaccatcagctcactgcagcctgacgacttcgcaacctactactgtctgcagtggagtagctacccttggacattcggcggcggcaccaaggtggagatcaag

SEQ ID NO: 2 - protein, 106 aa, Artificial Sequence
  Synthetic Construct [CDS]1..318 from SEQ ID NO 1
  Synthetic Construct
  Synthetic Construct
DIQMTQSPSTLSASVGDRVTITCRASSSVRYIAWYQQKPGKAPKLLTYPTSSLESGVPSRFSGSGSGTEFTLTISSLQPDDFATYYCLQWSSYPWTFGGGTKVEIK

SEQ ID NO: 3 - DNA, 345 bp, Artificial Sequence
  Synthetic 3C_23 VH without leader
  Synthetic 3C_23 VH without leader
  Synthetic 3C_23 VH without leader
  CDS 1..345
caggtgcggctggtgcagagcggggccgaggtgaagaagcctggagcctcagtgaaggtgagttgcaaggcctccggttacaccttcaccagctaccacatccactgggtcagacaggctcccggccagagactggagtggatgggctggatctaccctggagatgactccaccaagtactcccagaagttccagggtcgcgtgaccattaccagggacaccagcgcctccactgcctacatggagctgtcttccctgagatctgaggataccgcagtctactactgtacacggggggaccgctttgcttactgggggcagggcactctggtgaccgtctcgagc

SEQ ID NO: 4 - protein, 115 aa, Artificial Sequence
  Synthetic Construct [CDS]1..345 from SEQ ID NO 3
  Synthetic Construct
  Synthetic Construct
QVRLVQSGAEVKKPGASVKVSCKASGYTFTSYHIHWVRQAPGQRLEWMGWIYPGDDSTKYSQKFQGRVTITRDTSASTAYMELSSLRSEDTAVYYCTRGDRFAYWGQGTLVTVSS

SEQ ID NO: 5 - DNA, 318 bp, Artificial Sequence
  Synthetic 3C_23K VL without leader
  Synthetic 3C_23K VL without leader
  Synthetic 3C_23K VL without leader
  CDS 1..318
gacatccagatgacacagtccccatctaccctgtctgcttccgtgggagatcgggtgactatcacctgcagagcaagctcctccgtgaggtacatcgcttggtaccagcagaagccaggaaaggccccaaagctgctgacctacccaacctcctccctgaaatccggggtgcccagcagattctcaggcagtggctccggcaccgaattcaccctgaccatcagctcactgcagcctgacgacttcgcaacctactactgtctgcagtggagtagctacccttggacattcggcggcggcaccaaggtggagatcaag

SEQ ID NO: 6 - protein, 106 aa, Artificial Sequence
  Synthetic Construct [CDS]1..318 from SEQ ID NO 5
  Synthetic Construct
  Synthetic Construct
DIQMTQSPSTLSASVGDRVTITCRASSSVRYIAWYQQKPGKAPKLLTYPTSSLKSGVPSRFSGSGSGTEFTLTISSLQPDDFATYYCLQWSSYPWTFGGGTKVEIK

SEQ ID NO: 7 - DNA, 345 bp, Artificial Sequence; identical to SEQ ID NO: 3
  Synthetic 3C_23K VH without leader
  Synthetic 3C_23K VH without leader
  Synthetic 3C_23K VH without leader
  CDS 1..345

SEQ ID NO: 8 - protein, 115 aa, Artificial Sequence; identical to SEQ ID NO: 4
  Synthetic Construct [CDS]1..345 from SEQ ID NO 7
  Synthetic Construct
  Synthetic Construct

SEQ ID NO: 9 - DNA, 639 bp, Artificial Sequence
  Synthetic 3C_23 light chain without leader
  Synthetic 3C_23 light chain without leader
  Synthetic 3C_23 light chain without leader
  CDS 1..639
gacatccagatgacacagtccccatctaccctgtctgcttccgtgggagatcgggtgactatcacctgcagagcaagctcctccgtgaggtacatcgcttggtaccagcagaagccaggaaaggccccaaagctgctgacctacccaacctcctccctggaatccggggtgcccagcagattctcaggcagtggctccggcaccgaattcaccctgaccatcagctcactgcagcctgacgacttcgcaacctactactgtctgcagtggagtagctacccttggacattcggcggcggcaccaaggtggagatcaagcggaccgtcgccgcaccaagtgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 10 - protein, 213 aa, Artificial Sequence
  Synthetic Construct [CDS]1..639 from SEQ ID NO 9
  Synthetic Construct
  Synthetic Construct
DIQMTQSPSTLSASVGDRVTITCRASSSVRYIAWYQQKPGKAPKLLTYPTSSLESGVPSRFSGSGSGTEFTLTISSLQPDDFATYYCLQWSSYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 1335 bp, Artificial Sequence
  Synthetic 3C_23 heavy chain without leader
  Synthetic 3C_23 heavy chain without leader
  Synthetic 3C_23 heavy chain without leader
  CDS 1..1335
caggtgcggctggtgcagagcggggccgaggtgaagaagcctggagcctcagtgaaggtgagttgcaaggcctccggttacaccttcaccagctaccacatccactgggtcagacaggctcccggccagagactggagtggatgggctggatctaccctggagatgactccaccaagtactcccagaagttccagggtcgcgtgaccattaccagggacaccagcgcctccactgcctacatggagctgtcttccctgagatctgaggataccgcagtctactactgtacacggggggaccgctttgcttactgggggcagggcactctggtgaccgtctcgagcgccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 12 - protein, 445 aa, Artificial Sequence
  Synthetic Construct [CDS]1..1335 from SEQ ID NO 11
  Synthetic Construct
  Synthetic Construct
QVRLVQSGAEVKKPGASVKVSCKASGYTFTSYHIHWVRQAPGQRLEWMGWIYPGDDSTKYSQKFQGRVTITRDTSASTAYMELSSLRSEDTAVYYCTRGDRFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 639 bp, Artificial Sequence
  Synthetic 3C_23K light chain without leader
  Synthetic 3C_23K light chain without leader
  Synthetic 3C_23K light chain without leader
  CDS 1..639
gacatccagatgacacagtccccatctaccctgtctgcttccgtgggagatcgggtgactatcacctgcagagcaagctcctccgtgaggtacatcgcttggtaccagcagaagccaggaaaggccccaaagctgctgacctacccaacctcctccctgaaatccggggtgcccagcagattctcaggcagtggctccggcaccgaattcaccctgaccatcagctcactgcagcctgacgacttcgcaacctactactgtctgcagtggagtagctacccttggacattcggcggcggcaccaaggtggagatcaagcggaccgtcgccgcaccaagtgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 14 - protein, 213 aa, Artificial Sequence
  Synthetic Construct [CDS]1..639 from SEQ ID NO 13
  Synthetic Construct
  Synthetic Construct
DIQMTQSPSTLSASVGDRVTITCRASSSVRYIAWYQQKPGKAPKLLTYPTSSLKSGVPSRFSGSGSGTEFTLTISSLQPDDFATYYCLQWSSYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - DNA, 1335 bp, Artificial Sequence; identical to SEQ ID NO: 11
  Synthetic 3C_23K heavy chain without leader
  Synthetic 3C_23K heavy chain without leader
  Synthetic 3C_23K heavy chain without leader
  CDS 1..1335

SEQ ID NO: 16 - protein, 445 aa, Artificial Sequence; identical to SEQ ID NO: 12
  Synthetic Construct [CDS]1..1335 from SEQ ID NO 15
  Synthetic Construct
  Synthetic Construct

SEQ ID NO: 17 - protein, 17 aa, Homo sapiens
  signal peptide
  signal peptide
  signal peptide
MLGSLGLWALLPTAVEA

SEQ ID NO: 18 - protein, 556 aa, Homo sapiens
  Human AMHR-II lacking the signal peptide SEQ ID NO 17
  Human AMHR-II lacking the signal peptide SEQ ID NO 17
  Human AMHR-II lacking the signal peptide SEQ ID NO 17
PPNRRTCVFFEAPGVRGSTKTLGELLDTGTELPRAIRCLYSRCCFGIWNLTQDRAQVEMQGCRDSDEPGCESLHCDPSPRAHPSPGSTLFTCSCGTDFCNANYSHLPPPGSPGTPGSQGPQAAPGESIWMALVLLGLFLLLLLLLGSIILALLQRKNYRVRGEPVPEPRPDSGRDWSVELQELPELCFSQVIREGGHAVVWAGQLQGKLVAIKAFPPRSVAQFQAERALYELPGLQHDHIVRFITASRGGPGRLLSGPLLVLELHPKGSLCHYLTQYTSDWGSSLRMALSLAQGLAFLHEERWQNGQYKPGIAHRDLSSQNVLIREDGSCAIGDLGLALVLPGLTQPPAWTPTQPQGPAAIMEAGTQRYMAPELLDKTLDLQDWGMALRRADIYSLALLLWEILSRCPDLRPDSSPPPFQLAYEAELGNTPTSDELWALAVQERRRPYIPSTWRCFATDPDGLRELLEDCWDADPEARLTAECVQQRLAALAHPQESHPFPESCPRGCPPLCPEDCTSIPAPTILPCRPQRSACHFSVQQGPCSRNPQPACTLSPV

SEQ ID NO: 19 - protein, 115 aa, Artificial Sequence; identical to SEQ ID NO: 4
  Synthetic 3C23K/3C23
  Synthetic 3C23K/3C23
  Synthetic 3C23K/3C23

SEQ ID NO: 20 - protein, 115 aa, Artificial Sequence
  Synthetic 3C23KR/6B78
  Synthetic 3C23KR/6B78
  Synthetic 3C23KR/6B78
QVQLVQSGAEVKKPGASVKVSCKASGYTFTSYHIHWVRQAPGQRLEWMGWIYPGDDSTKYSQKFQGRVTITRDTSASTAYMELSSLRSEDTAVYYCTRGDRFAYWGQGTLVTVSS

SEQ ID NO: 21 - protein, 115 aa, Artificial Sequence
  Synthetic 5B42
  Synthetic 5B42
  Synthetic 5B42
QVQLVQSGAEVKKPGASVKVSCKASGYTFTSYHIHWVRQAPGQRLEWMGWIYPGDDSTKYSQKFQGRVTITRDASASTAYMELSSLRSEDTAVYYCTRGDRFAYWGQGTLVTVSS

SEQ ID NO: 22 - protein, 115 aa, Artificial Sequence
  Synthetic K4D-24/6C59
  Synthetic K4D-24/6C59
  Synthetic K4D-24/6C59
RVRLVQSGAEVKKPGASVKVSCKASGYTFTSYHIHWVRQAPGQRLEWMGWIYPGDDSTKYSQKFQGRVTITRDTSASTAYMELSSLRSEDTAVYYCTRGDRFAYWGQGTLVTVSS

SEQ ID NO: 23 - protein, 115 aa, Artificial Sequence
  Synthetic K4D-20
  Synthetic K4D-20
  Synthetic K4D-20
QVRLVQSGAEVKKPGASVKVSCKASGYTFTSNHIHWVRQAPGQRLEWMGWIYPGDDSTKYSQKFQGRVTITRDTSASTAYMELSSLRSEDTAVYYCTRGDRFAYWGQGTLVTVSS

SEQ ID NO: 24 - protein, 115 aa, Artificial Sequence
  Synthetic K4A-12
  Synthetic K4A-12
  Synthetic K4A-12
QVRLVQSGAEVKKPGTSVKVSCKASGYTFTSYHIHWVRQAPGQRLEWMGWIYPGDDSTKYSQKFQGRVTITRDTSASTAYMELSSLRSEDTAVYYCTRGDRFAYWGQGTLVTVSS

SEQ ID NO: 25 - protein, 115 aa, Artificial Sequence
  Synthetic K5D05
  Synthetic K5D05
  Synthetic K5D05
QVRLVQSGAEVKKPGASVKVSCKASGYTFTGYHIHWVRQAPGQRLEWMGWIYPGDDSTKYSQKFQGRVTITRDTSASTAYMELSSLRSEDTAVYYCTRGDRFAYWGQGTLVTVSS

SEQ ID NO: 26 - protein, 115 aa, Artificial Sequence
  Synthetic K5D-14
  Synthetic K5D-14
  Synthetic K5D-14
QVRLVQSGAEVKKPGASVKVSCKASGYSFTSYHIHWVRQAPGQRLEWMGWIYPGDDSTKYSQKFQGRVTITRDTSASTAYMELSSLRSEDTAVYYCTRGDRFAYWGQGTLVTVSS

SEQ ID NO: 27 - protein, 115 aa, Artificial Sequence
  Synthetic K4D-123
  Synthetic K4D-123
  Synthetic K4D-123
QVRLVQSGAEVKKPGASVKVSCKASGYTFTSYHIHWVRQAPGQSLEWMGWIYPGDDSTKYSQKFQGRVTITRDTSASTAYMELSSLRSEDTAVYYCTRGDRFAYWGQGTLVTVSS

SEQ ID NO: 28 - protein, 115 aa, Artificial Sequence
  Synthetic K4D-127/6C07
  Synthetic K4D-127/6C07
  Synthetic K4D-127/6C07
QVRLVQSGAEVKKPGASVKVSCKASGYTFTSYHIHWVRQAPGQRLEWMGWIYPGDDSTKYSQKFQGRVTTTRDTSASTAYMELSSLRSEDTAVYYCTRGDRFAYWGQGTLVTVSS

SEQ ID NO: 29 - protein, 115 aa, Artificial Sequence
  Synthetic 5C14
  Synthetic 5C14
  Synthetic 5C14
QVRLVQSGAEVKKPGASVKVSCKASGYTFTSYHIHWVRQAPGQRLEWMGWIYPGDDSTKYSQKFQGRVTFTRDTSASTAYMELSSLRSEDTAVYYCTRGDRFAYWGQGTLVTVSS

SEQ ID NO: 30 - protein, 115 aa, Artificial Sequence
  Synthetic 5C26
  Synthetic 5C26
  Synthetic 5C26
QVRLVQSGAEVKKPGASVKVSCKASGYTFTSYHIHWVRQAPGQRLEWMGWIYPGDDSTKYSQKFQGRMTITRDTSASTAYMELSSLRSEDTAVYYCTRGDRFAYWGQGTLVTVSS

SEQ ID NO: 31 - protein, 115 aa, Artificial Sequence
  Synthetic 5C27
  Synthetic 5C27
  Synthetic 5C27
QVRLVQSGAEVKKPGASVKVSCKASGYTFTSYHIHWVRQAPGQRPEWMGWIYPGDDSTKYSQKFQGRVTITRDTSASTAYMELSSLRSEDTAVYYCTRGDRFAYWGQGTLVTVSS

SEQ ID NO: 32 - protein, 115 aa, Artificial Sequence
  Synthetic 5C60
  Synthetic 5C60
  Synthetic 5C60
QVRLVQSGAKVRKPGASVKVSCKASGYTFTSYHIHWVRQAPGQRLEWMGWIYPGDDSTKYSQKFQGRVTITRDTSASTAYMELSSLRSEDTAVYYCTRGDRFAYWGQGTLVTVSS

SEQ ID NO: 33 - protein, 115 aa, Artificial Sequence
  Synthetic 6C13
  Synthetic 6C13
  Synthetic 6C13
QVRLVQSGAEVKKPGASVKVSCKASGYTFTSYHIHWVRQAPGQRLEWMGWIYPEDDSTKYSQKFQGRVTITRDTSASTAYMELSSLRSEDTAVYYCTRGDRFAYWGQGTLVTVSS

SEQ ID NO: 34 - protein, 115 aa, Artificial Sequence
  Synthetic 6C18
  Synthetic 6C18
  Synthetic 6C18
QVRLVQSGAEVKKPGASVKVSCKASGYTFTSYHIHWVRQAPGQRLEWMGWIYPGDDSTKYSQKFQGRVTITRDTSASTAYMELSSLRSEDTAVYYCARGDRFAYWGQGTLVTVSS

SEQ ID NO: 35 - protein, 115 aa, Artificial Sequence
  Synthetic 6C54
  Synthetic 6C54
  Synthetic 6C54
QVRLVQSGAEVKKPGASVKVSCKASGYTFTSYHIHWVRQAPGQRLEWMGWIYPGDDSTKYSQKFQGRVTITRDTSASTAYMELSSLRPEDTAVYYCTRGDRFAYWGQGTLVTVSS

SEQ ID NO: 36 - protein, 106 aa, Artificial Sequence; identical to SEQ ID NO: 6
  Synthetic 3C23K
  Synthetic 3C23K
  Synthetic 3C23K

SEQ ID NO: 37 - protein, 106 aa, Artificial Sequence; identical to SEQ ID NO: 2
  Synthetic L-K55E
  Synthetic L-K55E
  Synthetic L-K55E

SEQ ID NO: 38 - protein, 106 aa, Artificial Sequence
  Synthetic L-T48I, L-P50S
  Synthetic L-T48I, L-P50S
  Synthetic L-T48I, L-P50S
DIQMTQSPSTLSASVGDRVTITCRASSSVRYIAWYQQKPGKAPKLLIYSTSSLKSGVPSRFSGSGSGTEFTLTISSLQPDDFATYYCLQWSSYPWTFGGGTKVEIK

SEQ ID NO: 39 - protein, 106 aa, Artificial Sequence
  Synthetic LT48I, L-K55E
  Synthetic LT48I, L-K55E
  Synthetic LT48I, L-K55E
DIQMTQSPSTLSASVGDRVTITCRASSSVRYIAWYQQKPGKAPKLLIYPTSSLESGVPSRFSGSGSGTEFTLTISSLQPDDFATYYCLQWSSYPWTFGGGTKVEIK

SEQ ID NO: 40 - protein, 106 aa, Artificial Sequence
  Synthetic LS27P, L-S28P
  Synthetic LS27P, L-S28P
  Synthetic LS27P, L-S28P
DIQMTQSPSTLSASVGDRVTITCRASPPVRYIAWYQQKPGKAPKLLTYPTSSLKSGVPSRFSGSGSGTEFTLTISSLQPDDFATYYCLQWSSYPWTFGGGTKVEIK

SEQ ID NO: 41 - protein, 106 aa, Artificial Sequence
  Synthetic L-M4L, L-T20A
  Synthetic L-M4L, L-T20A
  Synthetic L-M4L, L-T20A
DIQLTQSPSTLSASVGDRVAITCRASSSVRYIAWYQQKPGKAPKLLTYPTSSLKSGVPSRFSGSGSGTEFTLTISSLQPDDFATYYCLQWSSYPWTFGGGTKVEIK

SEQ ID NO: 42 - protein, 106 aa, Artificial Sequence
  Synthetic L-S27P
  Synthetic L-S27P
  Synthetic L-S27P
DIQMTQSPSTLSASVGDRVTITCRASPSVRYIAWYQQKPGKAPKLLTYPTSSLKSGVPSRFSGSGSGTEFTLTISSLQPDDFATYYCLQWSSYPWTFGGGTKVEIK

SEQ ID NO: 43 - protein, 106 aa, Artificial Sequence
  Synthetic L-M4L, L-S9P, L-R31W
  Synthetic L-M4L, L-S9P, L-R31W
  Synthetic L-M4L, L-S9P, L-R31W
DIQLTQSPPTLSASVGDRVTITCRASSSVWYIAWYQQKPGKAPKLLTYPTSSLKSGVPSRFSGSGSGTEFTLTISSLQPDDFATYYCLQWSSYPWTFGGGTKVEIK

SEQ ID NO: 44 - protein, 106 aa, Artificial Sequence
  Synthetic L-M4L
  Synthetic L-M4L
  Synthetic L-M4L
DIQLTQSPSTLSASVGDRVTITCRASSSVRYIAWYQQKPGKAPKLLTYPTSSLKSGVPSRFSGSGSGTEFTLTISSLQPDDFATYYCLQWSSYPWTFGGGTKVEIK

SEQ ID NO: 45 - protein, 106 aa, Artificial Sequence
  Synthetic L-I33T
  Synthetic L-I33T
  Synthetic L-I33T
DIQMTQSPSTLSASVGDRVTITCRASSSVRYTAWYQQKPGKAPKLLTYPTSSLKSGVPSRFSGSGSGTEFTLTISSLQPDDFATYYCLQWSSYPWTFGGGTKVEIK

SEQ ID NO: 46 - protein, 106 aa, Artificial Sequence
  Synthetic L-M4L, L-K39E
  Synthetic L-M4L, L-K39E
  Synthetic L-M4L, L-K39E
DIQLTQSPSTLSASVGDRVTITCRASSSVRYIAWYQQEPGKAPKLLTYPTSSLKSGVPSRFSGSGSGTEFTLTISSLQPDDFATYYCLQWSSYPWTFGGGTKVEIK

SEQ ID NO: 47 - protein, 106 aa, Artificial Sequence
  Synthetic L-T22P
  Synthetic L-T22P
  Synthetic L-T22P
DIQMTQSPSTLSASVGDRVTIPCRASSSVRYIAWYQQKPGKAPKLLTYPTSSLKSGVPSRFSGSGSGTEFTLTISSLQPDDFATYYCLQWSSYPWTFGGGTKVEIK

SEQ ID NO: 48 - protein, 106 aa, Artificial Sequence
  Synthetic L-Y32D
  Synthetic L-Y32D
  Synthetic L-Y32D
DIQMTQSPSTLSASVGDRVTITCRASSSVRDIAWYQQKPGKAPKLLTYPTSSLKSGVPSRFSGSGSGTEFTLTISSLQPDDFATYYCLQWSSYPWTFGGGTKVEIK

SEQ ID NO: 49 - protein, 106 aa, Artificial Sequence
  Synthetic L-Q37H
  Synthetic L-Q37H
  Synthetic L-Q37H
DIQMTQSPSTLSASVGDRVTITCRASSSVRYIAWYHQKPGKAPKLLTYPTSSLKSGVPSRFSGSGSGTEFTLTISSLQPDDFATYYCLQWSSYPWTFGGGTKVEIK

SEQ ID NO: 50 - protein, 106 aa, Artificial Sequence
  Synthetic L-G97S
  Synthetic L-G97S
  Synthetic L-G97S
DIQMTQSPSTLSASVGDRVTITCRASSSVRYIAWYQQKPGKAPKLLTYPTSSLKSGVPSRFSGSGSGTEFTLTISSLQPDDFATYYCLQWSSYPWTFSGGTKVEIK

SEQ ID NO: 51 - protein, 106 aa, Artificial Sequence
  Synthetic L-S12P
  Synthetic L-S12P
  Synthetic L-S12P
DIQMTQSPSTLPASVGDRVTITCRASSSVRYIAWYQQKPGKAPKLLTYPTSSLKSGVPSRFSGSGSGTEFTLTISSLQPDDFATYYCLQWSSYPWTFGGGTKVEIK

SEQ ID NO: 52 - protein, 106 aa, Artificial Sequence
  Synthetic L-19A
  Synthetic L-19A
  Synthetic L-19A
DIQMTQSPSTLSASVGDRATITCRASSSVRYIAWYQQKPGKAPKLLTYPTSSLKSGVPSRFSGSGSGTEFTLTISSLQPDDFATYYCLQWSSYPWTFGGGTKVEIK

SEQ ID NO: 53 - protein, 106 aa, Artificial Sequence
  Synthetic L-T72A
  Synthetic L-T72A
  Synthetic L-T72A
DIQMTQSPSTLSASVGDRVTITCRASSSVRYIAWYQQKPGKAPKLLTYPTSSLKSGVPSRFSGSGSGTEFALTISSLQPDDFATYYCLQWSSYPWTFGGGTKVEIK

SEQ ID NO: 54 - protein, 106 aa, Artificial Sequence
  Synthetic L-R31W
  Synthetic L-R31W
  Synthetic L-R31W
DIQMTQSPSTLSASVGDRVTITCRASSSVWYIAWYQQKPGKAPKLLTYPTSSLKSGVPSRFSGSGSGTEFTLTISSLQPDDFATYYCLQWSSYPWTFGGGTKVEIK

SEQ ID NO: 55 - protein, 106 aa, Artificial Sequence
  Synthetic L-M4L, L-M39K
  Synthetic L-M4L, L-M39K
  Synthetic L-M4L, L-M39K
DIQLTQSPSTLSASVGDRVTITCRASSSVRYIAWYQQMPGKAPKLLTYPTSSLKSGVPSRFSGSGSGTEFTLTISSLQPDDFATYYCLQWSSYPWTFGGGTKVEIK

SEQ ID NO: 56 - protein, 106 aa, Artificial Sequence
  Synthetic L-I2N
  Synthetic L-I2N
  Synthetic L-I2N
DNQMTQSPSTLSASVGDRVTITCRASSSVRYIAWYQQKPGKAPKLLTYPTSSLKSGVPSRFSGSGSGTEFTLTISSLQPDDFATYYCLQWSSYPWTFGGGTKVEIK

SEQ ID NO: 57 - protein, 106 aa, Artificial Sequence
  Synthetic L-G63C, L-W91C
  Synthetic L-G63C, L-W91C
  Synthetic L-G63C, L-W91C
DIQMTQSPSTLSASVGDRVTITCRASSSVRYIAWYQQKPGKAPKLLTYPTSSLKSGVPSRFSCSGSGTEFTLTISSLQPDDFATYYCLQCSSYPWTFGGGTKVEIK

SEQ ID NO: 58 - protein, 106 aa, Artificial Sequence
  Synthetic L-R31G
  Synthetic L-R31G
  Synthetic L-R31G
DIQMTQSPSTLSASVGDRVTITCRASSSVGYIAWYQQKPGKAPKLLTYPTSSLKSGVPSRFSGSGSGTEFTLTISSLQPDDFATYYCLQWSSYPWTFGGGTKVEIK

SEQ ID NO: 59 - protein, 106 aa, Artificial Sequence
  Synthetic L-I75F
  Synthetic L-I75F
  Synthetic L-I75F
DIQMTQSPSTLSASVGDRVTITCRASSSVRYIAWYQQKPGKAPKLLTYPTSSLKSGVPSRFSGSGSGTEFTLTFSSLQPDDFATYYCLQWSSYPWTFGGGTKVEIK

SEQ ID NO: 60 - protein, 106 aa, Artificial Sequence
  Synthetic L-I2T
  Synthetic L-I2T
  Synthetic L-I2T
DTQMTQSPSTLSASVGDRVTITCRASSSVRYIAWYQQKPGKAPKLLTYPTSSLKSGVPSRFSGSGSGTEFTLTISSLQPDDFATYYCLQWSSYPWTFGGGTKVEIK

SEQ ID NO: 61 - protein, 106 aa, Artificial Sequence
  Synthetic L-I2T, L-K42R
  Synthetic L-I2T, L-K42R
  Synthetic L-I2T, L-K42R
DTQMTQSPSTLSASVGDRVTITCRASSSVRYIAWYQQKPGRAPKLLTYPTSSLKSGVPSRFSGSGSGTEFTLTISSLQPDDFATYYCLQWSSYPWTFGGGTKVEIK

SEQ ID NO: 62 - protein, 106 aa, Artificial Sequence
  Synthetic L-Y49H
  Synthetic L-Y49H
  Synthetic L-Y49H
DIQMTQSPSTLSASVGDRVTITCRASSSVRYIAWYQQKPGKAPKLLTHPTSSLKSGVPSRFSGSGSGTEFTLTISSLQPDDFATYYCLQWSSYPWTFGGGTKVEIK

SEQ ID NO: 63 - protein, 106 aa, Artificial Sequence
  Synthetic L-M4L, L-T20S, L-K39E
  Synthetic L-M4L, L-T20S, L-K39E
  Synthetic L-M4L, L-T20S, L-K39E
DIQLTQSPSTLSASVGDRVSITCRASSSVRYIAWYQQEPGKAPKLLTYPTSSLKSGVPSRFSGSGSGTEFTLTISSLQPDDFATYYCLQWSSYPWTFGGGTKVEIK

SEQ ID NO: 64 - protein, 106 aa, Artificial Sequence
  Synthetic L-T69P
  Synthetic L-T69P
  Synthetic L-T69P
DIQMTQSPSTLSASVGDRVTITCRASSSVRYIAWYQQKPGKAPKLLTYPTSSLKSGVPSRFSGSGSGPEFTLTISSLQPDDFATYYCLQWSSYPWTFGGGTKVEIK

SEQ ID NO: 65 - protein, 10 aa, Artificial Sequence
  Synthetic CDRL-1 of anti-AMHRII antibody
  Synthetic CDRL-1 of anti-AMHRII antibody
  VARIANT 4: Xaa in position 4 can be S or P
  VARIANT 5: Xaa in position 5 can be S or P
  VARIANT 7: Xaa in position 7 can be R or W or G
  VARIANT 8: Xaa in position 8 can be T or D
  VARIANT 9: Waa in position 9 can be I or T
RASXXVXXXA

SEQ ID NO: 66 - protein, 7 aa, Artificial Sequence
  Synthetic CDRL-2 of anti-AMHRII antibody
  Synthetic CDRL-2 of anti-AMHRII antibody
  VARIANT 6: Xaa in position 6 can be K or E
PTSSLXS

SEQ ID NO: 67 - protein, 9 aa, Artificial Sequence
  Synthetic CDRL-3 of anti-AMHRII antibody
  Synthetic CDRL-3 of anti-AMHRII antibody
LQWSSYPWT

SEQ ID NO: 68 - protein, 13 aa, Artificial Sequence
  Synthetic CDRH-1 of anti-AMHRII antibody
  Synthetic CDRH-1 of anti-AMHRII antibody
  VARIANT 6: Xaa in position 6 can be S or T
  VARIANT 9: Xaa in position 9 can be S or G
  VARIANT 10: Xaa in position 10 can be Y or N
KASGYXFTXXHIH

SEQ ID NO: 69 - protein, 17 aa, Artificial Sequence
  Synthetic CDRH-2 of anti-AMHRII antibody
  Synthetic CDRH-2 of anti-AMHRII antibody
  VARIANT 5: Xaa in position 5 can be G or E
WIYPXDDSTKYSQKFQG

SEQ ID NO: 70 - protein, 6 aa, Artificial Sequence
  Synthetic CDRH-3 of anti-AMHRII antibody
  Synthetic CDRH-3 of anti-AMHRII antibody
GDRFAY

SEQ ID NO: 71 - DNA, 20 bp, Artificial Sequence
  Synthetic formard primer for AMHR2
  Synthetic formard primer for AMHR2
tctggatggcactggtgctg

SEQ ID NO: 72 - DNA, 20 bp, Artificial Sequence
  Synthetic reverse primer for AMHR2
  Synthetic reverse primer for AMHR2
agcagggccaagatgatgct

SEQ ID NO: 73 - DNA, 21 bp, Artificial Sequence
  Synthetic forward primer for TBP
  Synthetic forward primer for TBP
tgcacaggagccaagagtgaa

SEQ ID NO: 74 - DNA, 20 bp, Artificial Sequence
  Synthetic reverse primer for TBP
  Synthetic reverse primer for TBP
cacatcacagctccccacca